(19) United States
(12) Patent Application Publication
(10) Pub. No.: US 2023/0323345 A1
(43) Pub. Date: Oct. 12, 2023

(54) UNA OLIGOMERS FOR THE TREATMENT OF POLYGLUTAMINE DISEASES

(71) Applicant: Arcturus Therapeutics, Inc., San Diego, CA (US)

(72) Inventors: Kiyoshi TACHIKAWA, San Diego, CA (US); Angel I-Jou LEU, San Diego, CA (US); Padmanabh CHIVUKULA, San Diego, CA (US); Priya Prakash KARMALI, San Diego, CA (US)

(21) Appl. No.: 18/001,571

(22) PCT Filed: Jun. 17, 2021
(86) PCT No.: PCT/US2021/037955

§ 371 Date: Dec. 12, 2022

Related U.S. Application Data
(60) Provisional application No. 63/040,949, filed on Jun. 18, 2020.

(51) Int. Cl. C12N 15/113; A61P 3/00

(52) U.S. Cl. CPC C12N 15/113; A61P 3/00; C12N 2310/11; C12N 2310/314; C12N 2310/315; C12N 2310/321; C12N 2310/3125; C12N 2310/3231

(57) ABSTRACT

An oligomer comprising a sense strand and an antisense strand that mediates RNA interference against a target RNA sequence having a trinucleotide repeat expansion is provided, wherein the antisense strand is complementary to the target RNA sequence and comprises a sequence having at least 80% identity to the sequence of Formula (I): rGrCrUrGrCrUrGrCX¹X²rCrUrGrCrUrGrCrUrG (I), wherein X¹ and X² are each independently selected from the group consisting of rA, rU, rG, rC, UNA-A, UNA-U, UNA-G, and UNA-C and wherein at least one of X¹ and X² is a UNA monomer; the oligomer comprises a UNA monomer at the first position at the 5′-end of the sense strand; and the sense strand and the antisense strand each independently include 19-29 monomers. The oligomers are useful as therapeutics targeting polyglutamine diseases and other diseases stemming from a trinucleotide repeat expansion.

### CROSS-REFERENCES TO RELATED APPLICATIONS

This application claims the benefit of U.S. Provisional Application No. 63/040,949, filed Jun. 18, 2020, which is incorporated herein by reference in its entirety and for all purposes.

### SEQUENCE LISTING

The instant application contains a Sequence Listing, which has been submitted electronically in ASCII format and is hereby incorporated by reference in its entirety. Said ASCII copy, created on Jun. 17, 2021 is named 049386-531001WO_SEQUENCE_LISTING_ST25.txt and is 6,234 bytes in size.

### TECHNICAL FIELD

The disclosure herein relates to the fields of biopharmaceuticals and therapeutics that are operable by gene silencing of trinucleotide repeats. More particularly, the disclosure relates to the structures, compositions and uses of siRNA (small interfering ribonucleic acid) and siRNA conjugates to mediate RNA interference against a target RNA sequence and/or modulate gene expression.

### BACKGROUND

Several neurological diseases are classified as polyglutamine diseases that are typically characterized by a late onset, with most manifestations not occurring until the affected subject’s 30s or 40s. Polyglutamine (polyQ) disease results from trinucleotide repeat expansion, a mutation that causes a polyglutamine tract in a specific gene to become abnormally long. Trinucleotide repeat expansion, also known as triplet repeat expansion, can be caused by slippage during DNA replication, also known as “copy choice” DNA replication. Due to the repetitive nature of the DNA sequence in these regions, “loop out” structures may form during DNA replication while maintaining complementary base pairing between the parent strand and daughter strand being replicated. If the loop out structure is formed from the sequence on the daughter strand, this will result in an increase in the number of repeats. However, if the loop out structure is formed on the parent strand, a decrease in the number of repeats occurs. Generally, the larger the expansion the more likely it will cause disease or increase the severity of disease. Another proposed mechanism for expansion and reduction involves the interaction of RNA and DNA molecules.

Polyglutamine disease involves CAGtrinucleotide repeats that cause neuronal degeneration characterized by abnormal protein folding and aggregation. Trinucleotide repeats may interfere with DNA structure, transcription, and RNA-protein interaction and may result in altered protein conformation and interactions. Abnormal protein conformation is supported by evidence that antibodies preferentially bind expanded polyQ, with in vitro studies showing that mutant polyQ self-associates into amyloid fibrils. (Paulson H.L., et al., PNAS, Nov. 21, 2000, Vol. 97, No. 24, pp. 12957-12958) Among the various diseases in the polyQ family, the trinucleotide repeat sequences CGG, GCC, GAA, CTG, and CAG are divided into two subclasses of trinucleotide repeat diseases, with a first subclass having repeats in non-coding sequences and the second subclass having CAG repeats coding for polyglutamine disease.

One promising therapeutic strategy is targeting the causative gene expression from genes that include trinucleotide repeat sequences. For example, the development of nucleic acids that can selectively target CAG repeat expansion that leads to polyQ disease is an emerging field of medicine that presents both great challenges and great potential in the treatment of disease. Among the possible treatment avenues using nucleic acids is the delivery of a small interfering RNA (siRNA), which interferes with the expression of a specific gene in a subject. However, for central nervous system (CNS) diseases such as PolyQ diseases, a major challenge is crossing the blood-brain barrier after systemic delivery of the nucleic acids. Moreover, siRNA-based therapies face several obstacles, including achieving an adequate in vivo half-life of the siRNA, achieving suitable levels of interference of gene expression by the siRNA or an siRNA conjugate, minimizing adverse reactions from the siRNA (e.g., poor selectivity), cellular uptake from the extracellular matrix, and reaching a specific cell compartment.

One method for delivering nucleic acids to target cells that has been successfully employed is the encapsulation of the nucleic acid in a lipid formulation such as a liposome or a lipid nanoparticle. While the use of lipid formulations has had some success, it has been found that several of the lipids used in these formulations show low in vivo degradability and low potency.

In light of the above challenges, novel approaches and therapies are still needed for the treatment of polyQ diseases, and strategies are needed that overcome the challenges and limitations associated with siRNA-based therapies, such as poor stability, poor cellular uptake from the extracellular matrix, and efficient delivery to the target cells, such as cells in the CNS.

Therefore, there is a continuing need for improved therapeutics for the treatment of polyQ diseases, for example, agents and effective delivery platforms that can selectively target CAG repeats that lead to polyQ disease.

### SUMMARY

Additional features and advantages of the subject technology will be set forth in the description below, and will be apparent from the following description, and/or may be learned by practice of the subject technology. The advantages of the subject technology will be realized and attained by the structures and compositions particularly pointed out in the written description and embodiments herein as well as the appended drawings.

It is to be understood that both the foregoing general description and the following detailed description are exemplary and explanatory and are intended to provide further explanation of the subject technology.

The present disclosure addresses the need for new therapeutic modalities by providing one or more small interfering RNA oligomers (siRNAs) that can act to knock down the activity of mutant polyQ proteins. These siRNAs are specially designed to target nucleotide sequences related to polyQ disorders. The presently disclosed siRNA oligomers are further enhanced by the presence of one or more modified ribonucleotide monomers, including UNA (unlocked nucleic acid), that are synergistically positioned within the siRNA sequences to provide enhanced knockdown activity and mutant selectivity. The experimental examples described herein show highly selective and effective knockdown activity of the presently disclosed siRNAs against more than one polyQ disease (e.g., SBMA, ATXN-3, HTT, etc.), which demonstrates that these siRNAs have a generalized ability to target polyQ diseases. In some embodiments, the oligomers target polyQ diseases characterized by CAG repeats.

In one aspect, disclosed herein is an oligomer comprising a sense strand and an antisense strand that mediates RNA interference against a target RNA sequence having a polyglutamine trinucleotide repeat expansion, wherein: a) the antisense strand is complementary to the target RNA sequence and comprises a sequence having at least 80% identity to the sequence of Formula (I): rGrCrUrGrCrUrGrCX¹X²rCrUrGrCrUrGrCrUrG (I), wherein X¹ and X² are each independently selected from the group consisting of rA, rU, rG, rC, UNA-A, UNA-U, UNA-G, and UNA-C and wherein at least one of X¹ and X² is a UNA monomer; b) the oligomer comprises a UNA monomer at the first position at the 5′-end of the sense strand; and c) the sense strand and the antisense strand each independently comprise 19-29 monomers.

In another aspect, disclosed herein is a pharmaceutical composition comprising a an oligomer comprising a sense strand and an antisense strand that mediates RNA interference against a target RNA sequence having a polyglutamine trinucleotide repeat expansion, wherein: a) the antisense strand is complementary to the target RNA sequence and comprises a sequence having at least 80% identity to the sequence of Formula (I): rGrCrUrGrCrUrGrCX¹X²rCrUrGrCrUrGrCrUrG (I), wherein X¹ and X² are each independently selected from the group consisting of rA, rU, rG, rC, UNA-A, UNA-U, UNA-G, and UNA-C and wherein at least one of X¹ and X² is a UNA monomer; b) the oligomer comprises a UNA monomer at the first position at the 5′-end of the sense strand; and c) the sense strand and the antisense strand each independently comprise 19-29 monomers, and a pharmaceutically acceptable carrier.

### BRIEF DESCRIPTION OF THE DRAWINGS

FIG. 1 illustrates the construction of Luciferase-ATXN3 fusion protein as described in Example 1.

FIG. 2 provides a bar graph depicting the effect of UNA oligomers containing one or more UNA monomers on knockdown of the ATXN3 reporter gene expression in vitro.

FIG. 3 provides a bar graph depicting the effect of UNA oligomers containing one or more UNA monomers on knockdown of the ATXN3 reporter gene expression in vitro.

FIG. 4 provides a bar graph depicting the effect of UNA oligomers containing one or more UNA monomers on knockdown of the ATXN3 reporter gene expression in vitro.

FIG. 5 shows the IC₅₀ data of UNA oligomers on knockdown of ATXN3 reporter gene expression in vitro and the selectivity for mutant over wild type (WT).

FIG. 6 shows western blot results of the knockdown (KD) effect of UNA oligomers on an androgen receptor expressed in fibroblasts from a healthy donor and a Spinobulbar Muscular Atrophy (SBMA) patient in vitro.

FIG. 7 shows a bar graph depicting the KD effect and selectivity of UNA oligomers for an androgen receptor expressed in fibroblasts from a healthy donor and a SBMA patient in vitro.

FIG. 8 shows western blot results of the KD effect of UNA oligomers on an androgen receptor with different copy numbers of CAG repeats expressed in fibroblasts from a healthy donor and a SBMA patient in vitro.

FIG. 9 shows a bar graph depicting the KD effect and selectivity of UNA siRNA for an androgen receptor with different copy numbers of CAG repeats expressed in fibroblasts from a healthy donor and a SBMA patient in vitro.

FIG. 10 shows a bar graph depicting E Densitometry quantitation of relative AR protein expression levels as described in Example 5.

FIG. 11 shows the western blot results of the KD effect of a UNA oligomer on huntingtin (HTT) expressed in fibroblasts from a Huntington’s disease patient in vitro.

FIG. 12 illustrates a scheme of the experiment described in Example 6 in which LIPID FORMULATION-eGFP mRNA (LF-eGFP mRNA;1800 ng) or vehicle were intracerebroventricularly (ICV) injected into mice. At P4, mice were sacrificed, and their brains were dissected.

FIG. 13 shows the western blot results of AR levels in temporal cortex and cerebellum of AR97Q mice.

FIG. 14 shows a bar graph depicting densitometry quantitation of AR97Q protein levels as described in Example 6 (n = 4 and *p<0.05).

FIG. 15 shows western blot results of AR levels in the temporal cortex and cerebellum of AR24Q mice.

FIG. 16 shows a bar graph depicting the densitometry quantitation of AR24Q protein levels as described in Example 6 (n = 4).

FIG. 17 illustrates a scheme of the experiment described in Example 7. LIPID FORMULATION-eGFP mRNA (500 or 1800 ng) or vehicle were intracerebroventricularly (ICV) injected into mice at P1. At P4 or P7, mice were sacrificed, and their brains were dissected.

FIG. 18 shows fluorescence imaging of eGFP at P4 and P7 by fluorescence stereomicroscopy. LIPID FORMULATION-eGFP mRNA (1800 ng) or vehicle-injected brains were photographed at the same time.

FIG. 19 shows imaging of a time course of eGFP expression after ICV injection of 1800 ng of LIPID FORMULATION-eGFP mRNA into the brains of mice.

FIG. 20 shows imaging of dose-dependent eGFP expression at P4 in brain tissue upon delivery of eGFP mRNA.

FIG. 21 shows an illustration of an atlas of P4 sagittal brain, which specifically indicates the coronal sections: (i) the olfactory bulb; (ii) the lateral ventricle; (iii) the hippocampus; and (iv) the temporal cortex.

FIG. 22 shows fluorescence imaging of eGFP (left) and stereoscopic imaging (right) in the slices of the indicated coronal section cut lines (i-iv).

FIG. 23 shows images of immunohistochemistry for eGFP in the indicated brain regions (Scale bar = 200 mm).

FIG. 24 shows western blot results of eGFP expression in brain regions and the spinal cord.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details.

In one aspect, disclosed herein is an oligomer comprising a sense strand and an antisense strand that mediates RNA interference against a target RNA sequence having a polyglutamine trinucleotide repeat expansion, wherein: a) the antisense strand is complementary to the target RNA sequence and comprises a sequence having at least 85% identity to the sequence of Formula (I): rGrCrUrGrCrUrGrCX¹X²rCrUrGrCrUrGrCrUrG (I), wherein X¹ and X² are each independently selected from the group consisting of rA, rU, rG, rC, UNA-A, UNA-U, UNA-G, and UNA-C and wherein at least one of X¹ and X² is a UNA monomer; b) the oligomer comprises a UNA monomer at the first position at the 5′-end of the sense strand; and c) the sense strand and the antisense strand each independently comprise 19-29 monomers. In one aspect, the sense and the antisense strand each independently consist of 19-29 monomers.

In some embodiments, the antisense strand comprises a sequence having at least 90% identity to the sequence of Formula (I). In some embodiments, the antisense strand comprises a sequence having at least 91% identity to the sequence of Formula (I). In some embodiments, the antisense strand comprises a sequence having at least 92% identity to the sequence of Formula (I). In some embodiments, the antisense strand comprises a sequence having at least 93% identity to the sequence of Formula (I). In some embodiments, the antisense strand comprises a sequence having at least 94% identity to the sequence of Formula (I). In some embodiments, the antisense strand comprises a sequence having at least 95% identity to the sequence of Formula (I). In some embodiments, the antisense strand comprises a sequence having at least 96% identity to the sequence of Formula (I). In some embodiments, the antisense strand comprises a sequence having at least 97% identity to the sequence of Formula (I). In some embodiments, the antisense strand comprises a sequence having at least 98% identity to the sequence of Formula (I). In some embodiments, the antisense strand comprises a sequence having at least 99% identity to the sequence of Formula (I).

In some embodiments, the sense strand and the antisense strand each comprise deoxy T at the first position and the second position from the 3′ end.

In some embodiments, the oligomer further comprises one or more nucleic acid monomer analogs selected from the group consisting of locked nucleic acids, phosphorothioates, phosphoramidates, methyl phosphonates, chiral-methyl phosphonates, 2′-O-methyl ribonucleotides and peptide-nucleic acids.

In some embodiments, the antisense strand is a guide strand for RNA interference and the senses strand is a passenger strand for RNA interference.

In some embodiments, X¹ or X² is UNA-A. In some embodiments, X¹ or X² is UNA-G. In some embodiments, X¹ or X² is UNA-U. In some embodiments, X¹ or X² is UNA-C.

In some embodiments, the oligomer has one or two overhangs. In some embodiments, the oligomer has at least one 3′-overhang. In some embodiments, the oligomer has at least one 5′-overhang.

In some embodiments, the oligomer has at least one blunt end.

In some embodiments, the oligomer is complementary to a target nucleotide sequence.

In some embodiments, the oligomer has reduced off-target effects as compared to an identical oligonucleotide with natural RNA monomers.

In some embodiments, the oligomer has increased or prolonged potency for gene silencing as compared to an identical oligonucleotide with natural RNA monomers.

In some embodiments, the sense and antisense strands are connected and form a duplex region with a loop at one end.

In some embodiments, the oligomer selectively inhibits mutant gene expression verses wild-type gene expression.

In some embodiments, the oligomer selectively inhibits mutant gene expression versus wild-type gene expression by a factor of at least 5-fold.

In some embodiments, further comprising at least one siRNA that is base-modified, sugar-modified, and/or linkage modified.

In some embodiments, the sense strand comprises a sequence of SEQ ID NO: 2. In some embodiments, the antisense strand comprises a sequence selected from SEQ ID NOs: 5-10. In some embodiments, the antisense strand comprises a sequence of SEQ ID NO: 10. In some embodiments, the sense strand comprises a sequence of SEQ ID NO: 2 and the antisense strand comprises a sequence selected from SEQ ID NOs: 4-14. In some embodiments, the sense strand comprises a sequence of SEQ ID NO: 2 and the antisense strand comprises a sequence selected from SEQ ID NOs: 4-10. In some embodiments, the sense strand comprises a sequence of SEQ ID NO: 2 and the antisense strand comprises a sequence of SEQ ID NO: 10. In some embodiments, the sense strand consists of a sequence of SEQ ID NO: 2. In some embodiments, the antisense strand consists of a sequence selected from SEQ ID NOs: 5-10. In some embodiments, the antisense strand consists of a sequence of SEQ ID NO: 10. In some embodiments, the sense strand consists of a sequence of SEQ ID NO: 2 and the antisense strand consists of a sequence selected from SEQ ID NOs: 4-14. In some embodiments, the sense strand consists of a sequence of SEQ ID NO: 2 and the antisense strand consists of a sequence selected from SEQ ID NOs: 4-10. In some embodiments, the sense strand consists of a sequence of SEQ ID NO: 2 and the antisense strand consists of a sequence of SEQ ID NO: 10.

In another aspect, the oligomer further comprises a compound of Formula (II)

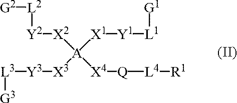

or a pharmaceutically acceptable salt or solvate thereof, wherein A is a tertiary carbon; X¹, X² and X³ are each independently selected from the group consisting of C₁₋C₁₀ alkyl, —(CH₂)ₘ—O—(CH₂)ₙ— and —(CH₂)ₘ—N—(CH₂)ₙ—, wherein n is 1-36 and m is 1-30; Y¹, Y² and Y³ are each independently selected from the group consisting of —NHC(O)—, —C(O)NH—, —OC(O)—, —C(O)O—,—SC(O)—, —C(O)S— and P(Z)(OH)O₂, wherein Z is O or S; L¹, L² and L³ are each independently selected from the group consisting of a C₁-C₁₀ alkyl, —(CH₂)ₑ—O—(CH₂)_{f}—, —(CH₂)ₑ—S—(CH₂)_{f}—, —(CH₂)ₑ—S(O)₂—(CH₂)_{f}—, —(CH₂)ₑ—N—(CH₂)_{f}— and —(CH₂—CH₂—O)ₖ(CH₂)₂—, wherein e is 1-10, f is 1-16; and k is 1-20; G¹, G² and G³ are each independently selected from the group consisting of a monosaccharide, a monosaccharide derivative, a vitamin, a polyol, a polysialic acid and a polysialic acid derivative; X⁴ is selected from the group consisting of (a) —(CH₂)_{g}—O—(CH₂)ₕ— or —(CH₂)_{g}—N—(CH₂)ₕ—, wherein g is 1-30 and h is 1-36, (b) an amino acid, and (c) —NHC(O)R², wherein R² is C₁-C₁₀ alkyl, a carbocycle, a heterocyclyl, a heteroaryl, a C₁-C₁₀ alkyl-carbocycle, a C₁-C₁₀ alkyl-heterocyclyl or a C₁-C₁₀ alkyl-heteroaryl, and wherein R² is optionally substituted; Q is absent, alkylamino, —C(O)—(CH₂)ᵢ—, —(CH₂)ᵢ—O—(CH₂)ⱼ—, —(CH₂)ᵢ—NR³—(CH₂)ⱼ—, —(CH₂)ᵢ—S—S—(CH₂)ⱼ—, —(CH₂)ᵢ—S—(CH₂)ⱼ—, —(CH₂)ᵢ—S(O)₂—(CH₂)ⱼ—_{,} —(CH₂)ᵢ—NHC(O)—(CH₂)ⱼ₋, —(CH₂)ᵢ—C(O)NH—(CH₂)ⱼ—, —(CH₂)ᵢ—SC(O)—(CH₂)ⱼ—, or —(CH₂)ᵢ—C(O)S—(CH₂)ⱼ—, wherein i is 1-30; j is 1-36; and R³ is hydrogen or an alkyl; L⁴ is absent, —C(O)O—, —C(O)NH—, a phosphate, C₁-C₁₀ alkyl-phosphate, C₂-C₁₀ alkenyl-phosphate, a phosphorothioate, C₁-C₁₀ alkyl-phosphorothioate, C₂-C₁₀ alkenyl-phosphorothioate, a boranophospate, a C₁-C₁₀ alkyl-boranophospate, a C₂-C₁₀ alkenyl-boranophospate, —C(O)NH—C₁-C₁₀alkyl-phosphate, —C(O)NH—C₂-C₁₀alkenyl-phosphate, —C(O)O—C₁-C₁₀alkyl-phosphate, —C(O)O—C₂-C₁₀alkenyl-phosphate, —C(O)NH—C₁-C₁₀alkyl-phosphorothioate, —C(O)NH—C₂-C₁₀alkenyl-phosphorothioate, —C(O)O—C₁-C₁₀alkyl-phosphorothioate, —C(O)O—C₂-C₁₀alkenyl-phosphorothioate, —C(O)—NH—C₁-C₁₀alkyl-boranophospate, —C(O)—NH—C₂-C₁₀alkenyl-boranophospate, —C(O)O—C₁-C₁₀alkyl-boranophospate or —C(O)O—C₂-C₁₀alkenyl-boranophospate; and R¹ is an oligomer comprising a sense strand and an antisense strand that mediates RNA interference as disclosed herein.

In some embodiments, X¹, X² and X³ are each independently (—CH₂)ₘ—O—CH₂—,wherein m is 1-4. In some embodiments, X¹, X² and X³ are each independently (—CH₂)₂—O—CH₂—.

In some embodiments, Y¹, Y² and Y³ are each —NHC(O)— or —C(O)NH—. In some embodiments, Y¹, Y² and Y³ are each —NHC(O)—.

In some embodiments, L¹, L² and L³ are each independently C₃-C₈ alkyl or —(CH₂—CH₂—O)ₖ(CH₂)₂—, wherein k is 1-10. In some embodiments, L¹, L² and L³ are each independently —(CH₂—CH₂—O)ₖ(CH₂)₂—, wherein k is 2-4. In some embodiments, L¹, L² and L³ are each C₁-C₁₀ alkyl.

In some embodiments, G¹, G² and G³ are each independently selected from the group consisting of folic acid, ribose, retinol, niacin, riboflavin, biotin, glucose, mannose, fucose, sucrose, lactose, mannose-6-phosphate, N-acetylgalactosamine, N-acetylglucosamine, a sialic acid, a sialic acid derivative, allose, altrose, arabinose, cladinose, erythrose, erythrulose, fructose, D-fucitol, L-fucitol, fucosamine, fucose, fuculose, galactosamine, D-galactosaminitol, galactose, glucosamine, glucosaminitol, glucose-6 phosphate, gulose glyceraldehyde, L-glycero-D-mannosheptose, glycerol, glycerone, gulose, idose, lyxose, mannosamine, psicose, quinovose, quinovosamine, rhamnitol, rhamnosamine, rhamnose, ribulose, sedoheptulose, sorbose, tagatose, talose, threose, xylose and xylulose. In some embodiments, G¹, G² and G³ are each N-acetylgalactosamine.

In some embodiments, X⁴ is selected from the group consisting of

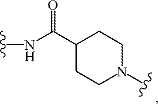

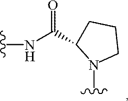

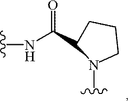

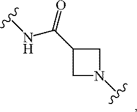

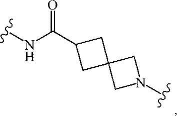

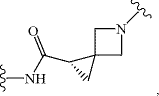

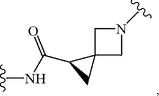

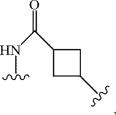

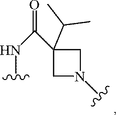

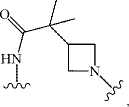

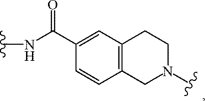

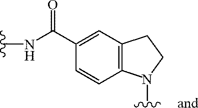

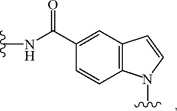

wherein X⁴ is optionally substituted.

In some embodiments, X⁴ is —NHC(O)R²; R² is a carbocycle, a heterocyclyl or a heteroaryl; and R² is optionally substituted; and Q is alkylamino, —C(O)—(CH₂)ᵢ—, —(CH₂)ᵢ—O—(CH₂)ⱼ—, —(CH₂)ᵢ—NR³—(CH₂)ⱼ—, —(CH₂)ᵢ—S—S—(CH₂)ⱼ—, —(CH₂)ᵢ—S—(CH₂)ⱼ—, —(CH₂)ᵢ—S(O)₂—(CH₂)ⱼ—, —(CH₂)ᵢ—NHC(O)—(CH₂)ⱼ—, —(CH₂)ᵢ—C(O)NH—(CH₂)ⱼ—, —(CH₂)ᵢ—SC(O)—(CH₂)ⱼ—, or —(CH₂)ᵢ—C(O)S—(CH₂)ⱼ—, wherein i is 1-10; j is 1-10; and R³ is hydrogen or an alkyl.

In some embodiments, X⁴ is

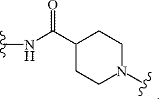

In some embodiments, Q is —C(O)—(CH₂)₁₋₁₀- and L⁴ is a —C(O)NH—(CH₂)₁₋₁₀-phosphate. In some embodiments, Q is —C(O)—(CH₂)₃— and L⁴ is a —C(O)NH—(CH₂)₆—phosphate. In some embodiments, L⁴ is —C(O)O—. In some embodiments, L⁴ is a —C(O)NH—(CH₂)₁₋₁₀-phosphate.

In some embodiments, the compound of Formula (II) has the formula:

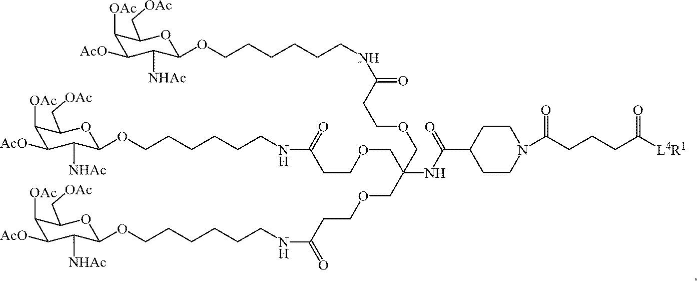

wherein R¹ is an oligomer comprising a sense strand and an antisense strand that mediates RNA interference as disclosed herein.

In some embodiments, the compound of Formula (II) is selected from the group consisting of

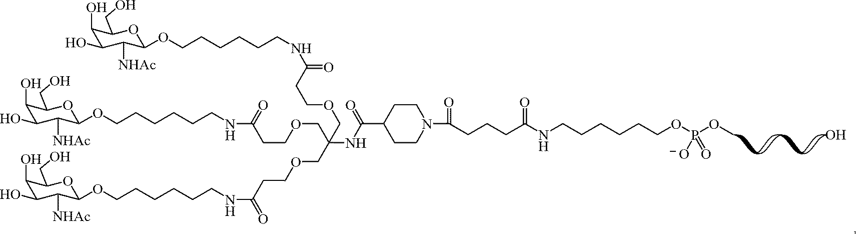

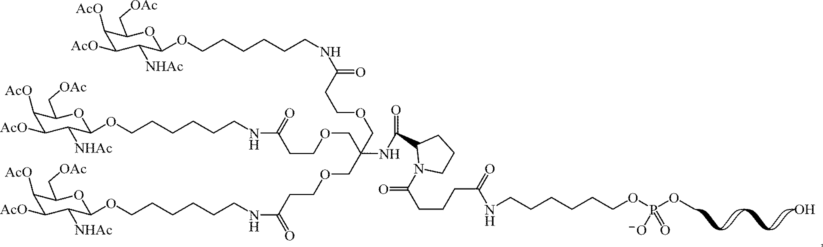

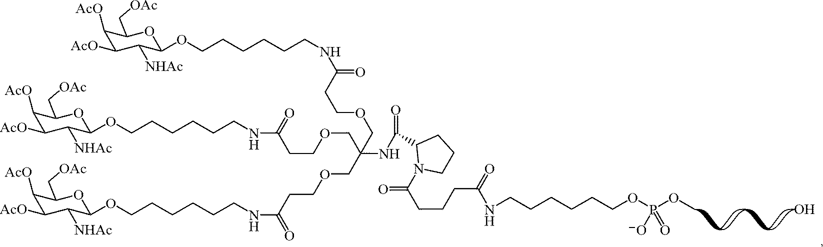

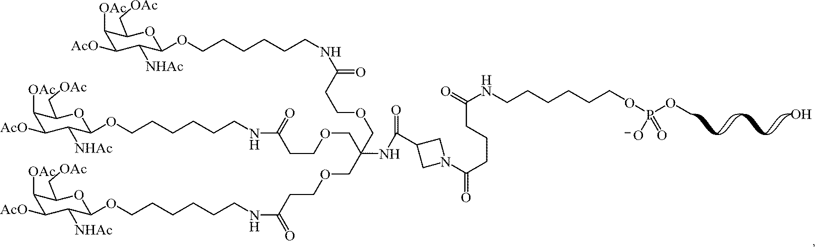

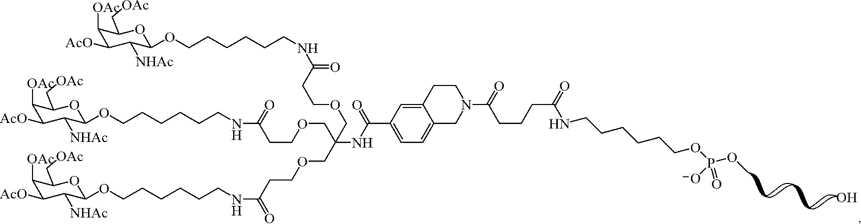

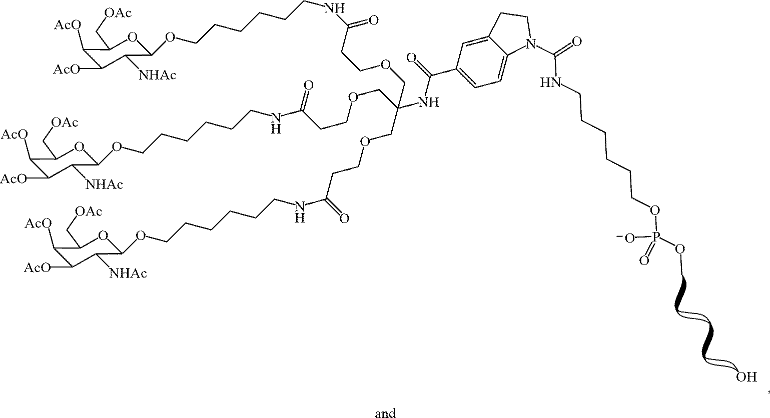

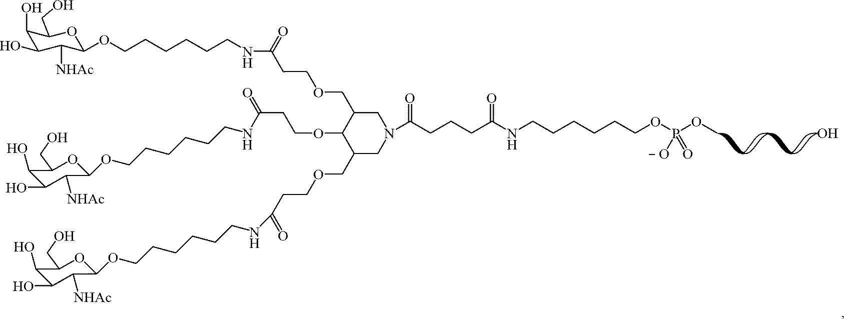

wherein

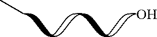

is an oligomer comprising a sense strand and an antisense strand that mediates RNA interference as disclosed herein.

In another aspect, the oligomer comprises a compound of Formula (III)

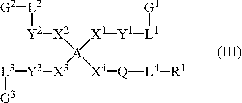

or a pharmaceutically acceptable salt or solvate thereof, wherein A is a tertiary carbon; X¹, X² and X³ are each independently selected from the group consisting of C₁-C₁₀ alkyl, —(CH₂)ₘ—O—(CH₂)ₙ— and —(CH₂)ₘ—N—(CH₂)ₙ—, wherein n is 1-36 and m is 1-30; Y¹, Y² and Y³ are each independently selected from the group consisting of —NHC(O)—, —C(O)NH—, —OC(O)—, —C(O)O—,—SC(O)—, —C(O)S— and P(Z)(OH)O₂, wherein Z is O or S; L¹, L² and L³ are each independently selected from the group consisting of a C₁-C₁₀ alkyl, —(CH₂)ₑ—O—(CH₂)_{f}—, —(CH₂)ₑ—S—(CH₂)_{f}—, —(CH₂)ₑ—S(O)₂—(CH₂)_{f}—, —(CH₂)ₑ—N—(CH₂)_{f}— and —(CH₂—CH₂—O)ₖ(CH₂)₂—, wherein e is 1-10; f is 1-16; and k is 1-20; G¹, G² and G³ are each independently selected from the group consisting of a monosaccharide, a monosaccharide derivative, a vitamin, a polyol, a polysialic acid and a polysialic acid derivative; X⁴ is selected from the group consisting of —(CH₂)_{g}—O—(CH₂)ₕ— or —(CH₂)_{g}—N—(CH₂)ₕ—, wherein g is 1-30 and h is 1-36, (a) an amino acid, and (b) —NHC(O)R², wherein R² is C₁-C₁₀ alkyl, a carbocycle, a heterocyclyl, a heteroaryl, a C₁-C₁₀ alkyl-carbocycle, a C₁-C₁₀ alkyl-heterocyclyl or a C₁-C₁₀ alkyl-heteroaryl, and wherein R² is optionally substituted; Q is alkylamino, —C(O)—(CH₂)ᵢ—, —(CH₂)ᵢ—O—(CH₂)ⱼ—, —(CH₂)ᵢ—NR³—(CH₂)ⱼ—, —(CH₂)ᵢ—S—S—(CH₂)ⱼ—, —(CH₂)ᵢ—S—(CH₂)ⱼ—, —(CH₂)ᵢ—S(O)₂—(CH₂)ⱼ—, —(CH₂)ᵢ—NHC(O)—(CH₂)ⱼ₋, —(CH₂)ᵢ—C(O)NH—(CH₂)ⱼ—, —(CH₂)ᵢ—SC(O)—(CH₂)ⱼ—, —(CH₂)ᵢ—C(O)S—(CH₂)ⱼ—, or

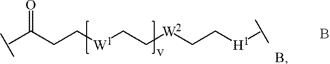

wherein H¹ is a carbocycle, a heterocyclyl or a heteroaryl; H¹ is optionally substituted; i is 1-30 and j is 1-36; R³ is hydrogen or an alkyl; W¹ and W² are each independently selected from —CH₂— and O; v is 1-6; Y is hydrogen or methyl; and T is C₁-C₁₀ alkyl or C₂-C₁₀ alkenyl; L⁴ is —C(O)O—, —C(O)NH—, a phosphate, C₁-C₁₀ alkyl-phosphate, C₂-C₁₀ alkenyl-phosphate, a phosphorothioate, C₁-C₁₀ alkyl-phosphorothioate, C₂-C₁₀ alkenyl-phosphorothioate, a boranophospate, a C₁-C₁₀ alkyl-boranophospate, a C₂-C₁₀ alkenyl-boranophospate, —C(O)NH—C₁-C₁₀alkyl-phosphate, —C(O)NH—C₂-C₁₀alkenyl-phosphate, —C(O)O—C₁-C₁₀alkyl-phosphate, —C(O)O—C₂-C₁₀alkenyl-phosphate, —C(O)NH—C₁-C₁₀alkyl-phosphorothioate, —C(O)NH—C₂-C₁₀alkenyl-phosphorothioate, —C(O)O—C₁-C₁₀alkyl-phosphorothioate, —C(O)O—C₂-C₁₀alkenyl-phosphorothioate, —C(O)—NH—C₁-C₁₀alkyl-boranophospate, —C(O)—NH—C₂-C₁₀alkenyl-boranophospate, —C(O)O—C₁-C₁₀alkyl-boranophospate or —C(O)O—C₂-C₁₀alkenyl-boranophospate; and R¹ is an oligomer comprising a sense strand and an antisense strand that mediates RNA interference as disclosed herein.

In another aspect, the oligomer comprises a compound of Formula (IV)

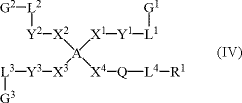

or a pharmaceutically acceptable salt or solvate thereof, wherein A is a tertiary carbon; X¹, X² and X³ are each independently selected from the group consisting of C₁-C₁₀ alkyl, —(CH₂)ₘ—O—(CH₂)ₙ— and —(CH₂)ₘ—N—(CH₂)ₙ—, wherein n is 1-36 and m is 1-30; Y¹, Y² and Y³ are each independently selected from the group consisting of —NHC(O)—, —C(O)NH—, —OC(O)—, —C(O)O—,—SC(O)—, —C(O)S— and P(Z)(OH)O₂, wherein Z is O or S; L¹, L² and L³ are each independently selected from the group consisting of a C₁-C₁₀ alkyl, —(CH₂)ₑ—O—(CH₂)_{f}—, —(CH₂)ₑ—S—(CH₂)_{f}—, —(CH₂)ₑ—S(O)₂—(CH₂)_{f}—, —(CH₂)ₑ—N—(CH₂)_{f}— and —(CH₂—CH₂—O)ₖ(CH₂)₂—, wherein e is 1-10, f is 1-16 and k is 1-20; G¹, G² and G³ are each independently selected from the group consisting of a monosaccharide, a monosaccharide derivative, a vitamin, a polyol, a polysialic acid and a polysialic acid derivative; X⁴ is selected from the group consisting of (a) —(CH₂)_{g}—O—(CH₂)ₕ— or —(CH₂)_{g}—N—(CH₂)ₕ—, wherein g is 1-30 and h is 1-36, (b) an amino acid, and (c) —NHC(O)R², wherein R² is C₁-C₁₀ alkyl, a carbocycle, a heterocyclyl, a heteroaryl, a C₁-C₁₀ alkyl-carbocycle, a C₁-C₁₀ alkyl-heterocyclyl or a C₁-C₁₀ alkyl-heteroaryl, and wherein R² is optionally substituted; Q is

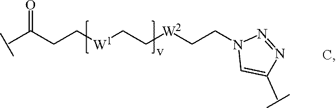

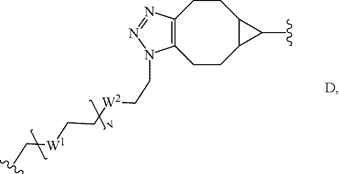

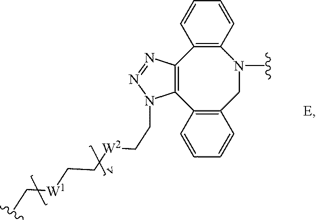

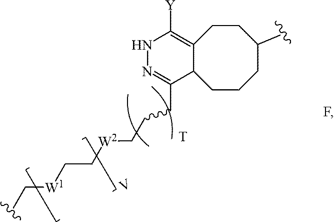

wherein H¹ is a carbocycle, a heterocyclyl or a heteroaryl; H¹ is optionally substituted; W¹ and W² are each independently selected from —CH₂— and O; v is 1-6; wherein Y is hydrogen or methyl; and T is C₁-C₁₀ alkyl or C₁-C₁₀ alkenyl; L⁴ is —C(O)O—, —C(O)NH—, a phosphate, C₁-C₁₀ alkyl-phosphate, C₂-C₁₀ alkenyl-phosphate, a phosphorothioate, C₁-C₁₀ alkyl-phosphorothioate, C₂-C₁₀ alkenyl-phosphorothioate, a boranophospate, a C₁-C₁₀ alkyl-boranophospate, a C₂-C₁₀ alkenyl-boranophospate, —C(O)NH—C₁-C₁₀alkyl-phosphate, —C(O)NH—C₂-C₁₀alkenyl-phosphate, —C(O)O—C₁-C₁₀alkyl-phosphate, —C(O)O—C₂-C₁₀alkenyl-phosphate, —C(O)NH—C₁-C₁₀alkyl-phosphorothioate, —C(O)NH—C₂-C₁₀alkenyl-phosphorothioate, —C(O)O—C₁-C₁₀alkyl-phosphorothioate, —C(O)O—C₂-C₁₀alkenyl-phosphorothioate, —C(O)—NH—C₁-C₁₀alkyl-boranophospate, —C(O)—NH—C₂-C₁₀alkenyl-boranophospate, —C(O)O—C₁-C₁₀alkyl-boranophospate or —C(O)O—C₂-C₁₀alkenyl-boranophospate; and R¹ is an oligomer comprising a sense strand and an antisense strand that mediates RNA interference as disclosed herein.

In some embodiments, X¹, X² and X³ are each independently (—CH₂)ₘ—O—CH₂—,wherein m is 1-4. In some embodiments, X¹, X² and X³ are each independently (—CH₂)₂—O—CH₂—.

In some embodiments, Y¹, Y² and Y³ are each —NHC(O)— or —C(O)NH—. In some embodiments, Y¹, Y² and Y³ are each —NHC(O)—.

In some embodiments, L¹, L² and L³ are each independently C₃-C₈ alkyl or —(CH₂—CH₂—O)ₖ(CH₂)₂—, wherein k is 1-10. In some embodiments, L¹, L² and L³ are each independently —(CH₂—CH₂—O)ₖ(CH₂)₂—, wherein k is 2-4. In some embodiments, L¹, L² and L³ are each C₁-C₁₀ alkyl.

In some embodiments, G¹, G² and G³ are each independently selected from the group consisting of folic acid, ribose, retinol, niacin, riboflavin, biotin, glucose, mannose, fucose, sucrose, lactose, mannose-6-phosphate, N-acetyl galactosamine, N-acetylglucosamine, a sialic acid, a sialic acid derivative, allose, altrose, arabinose, cladinose, erythrose, erythrulose, fructose, D-fucitol, L-fucitol, fucosamine, fucose, fuculose, galactosamine, D-galactosaminitol, galactose, glucosamine, glucosaminitol, glucose-6 phosphate, gulose glyceraldehyde, L-glycero-D-mannosheptose, glycerol, glycerone, gulose, idose, lyxose, mannosamine, psicose, quinovose, quinovosamine, rhamnitol, rhamnosamine, rhamnose, ribulose, sedoheptulose, sorbose, tagatose, talose, threose, xylose and xylulose. In some embodiments, G¹, G² and G³ are each N-acetylgalactosamine.

In some embodiments, X⁴ is selected from the group consisting of

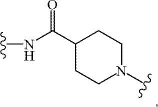

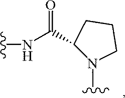

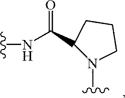

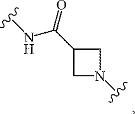

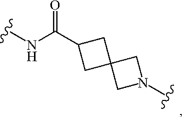

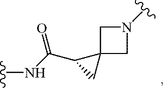

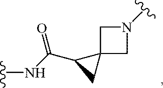

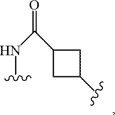

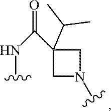

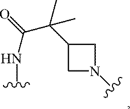

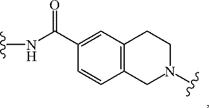

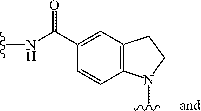

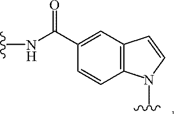

wherein X⁴ is optionally substituted.

In some embodiments, X⁴ is —NHC(O)R², wherein R² is a carbocycle, a heterocyclyl or a heteroaryl, wherein R² is optionally substituted; and Q is alkylamino, —C(O)—(CH₂)ᵢ—, —(CH₂)ᵢ—O—(CH₂)ⱼ—, —(CH₂)ᵢ—NR³—(CH₂)ⱼ—, —(CH₂)ᵢ—S—S—(CH₂)ⱼ—, —(CH₂)ᵢ—S—(CH₂)ⱼ—, —(CH₂)ᵢ—S(O)₂—(CH₂)ⱼ—, —(CH₂)ᵢ—NHC(O)—(CH₂)ⱼ₋, —(CH₂)ᵢ—C(O)NH—(CH₂)ⱼ—, —(CH₂)ᵢ—SC(O)—(CH₂)ⱼ—, or —(CH₂)ᵢ—C(O)S—(CH₂)ⱼ—, wherein i is 1-10 and j is 1-10, and wherein R³ is hydrogen or an alkyl.

In some embodiments, X⁴ is

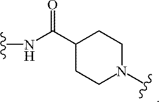

In some embodiments, the compound of Formula (IV) has the formula

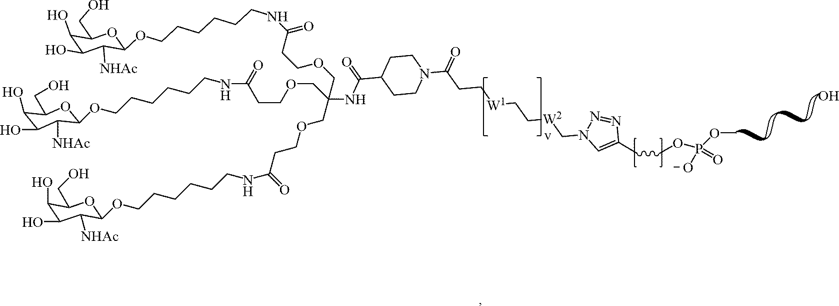

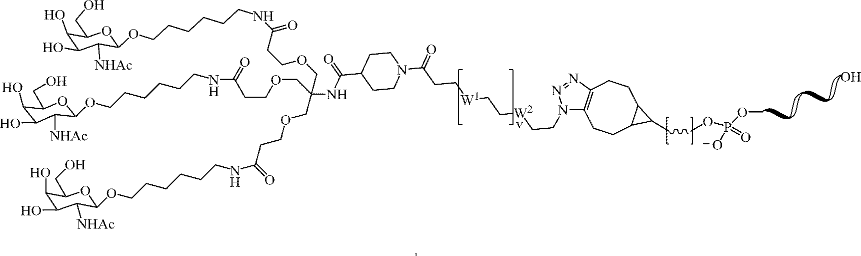

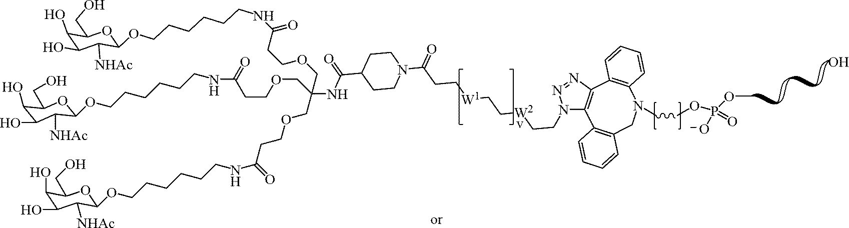

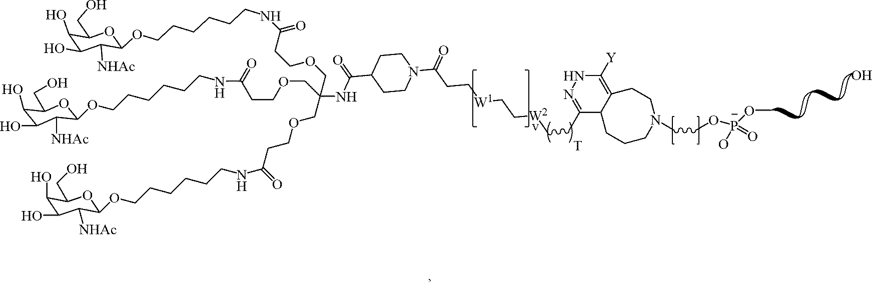

wherein

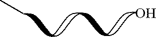

is an oligomer comprising a sense strand and an antisense strand that mediates RNA interference as disclosed herein, and

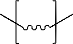

is C₁-C₁₀ alkyl or C₂-C₁₀ alkenyl.

In some embodiments, the compound of Formula (IV) has the formula

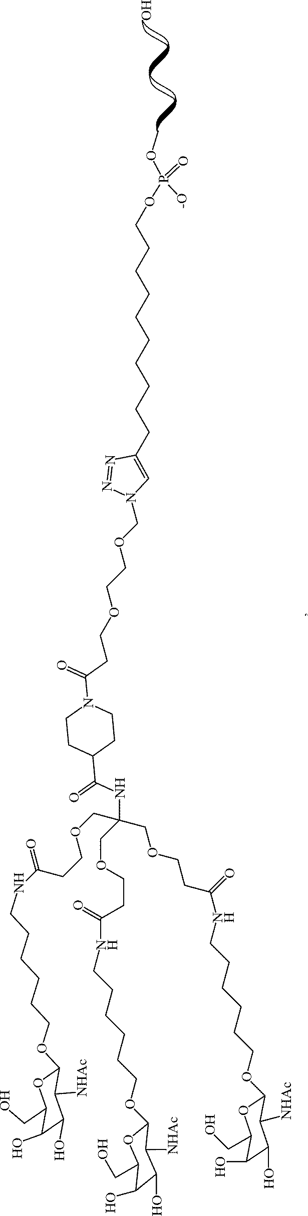

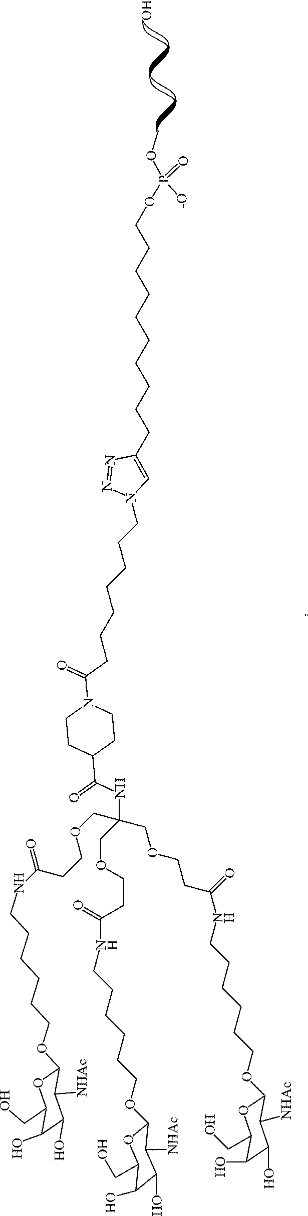

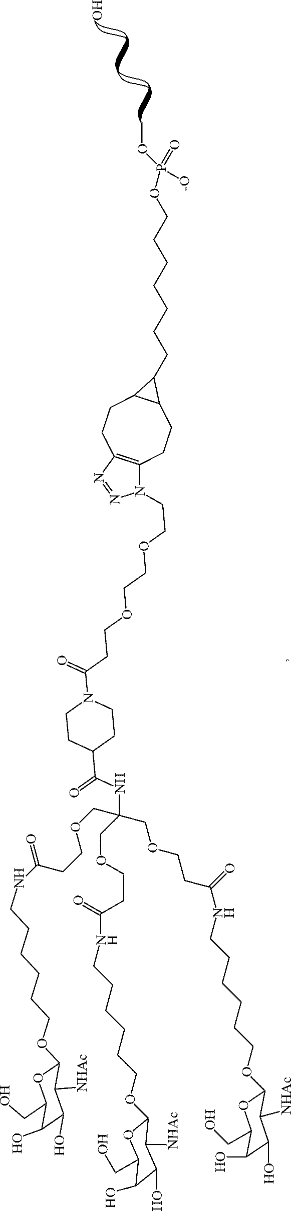

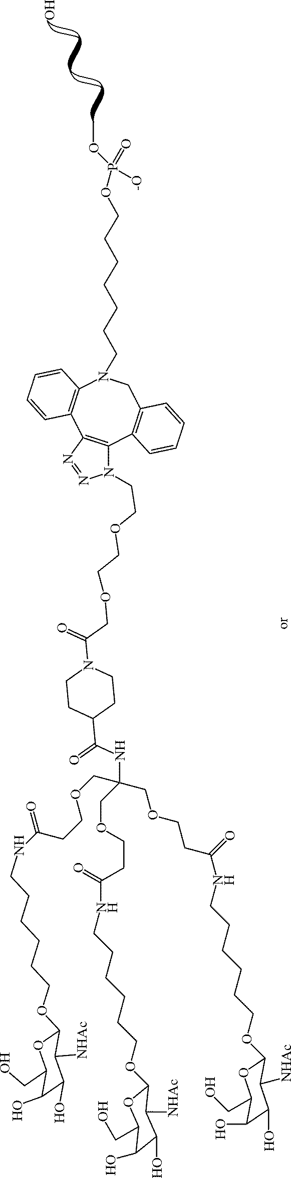

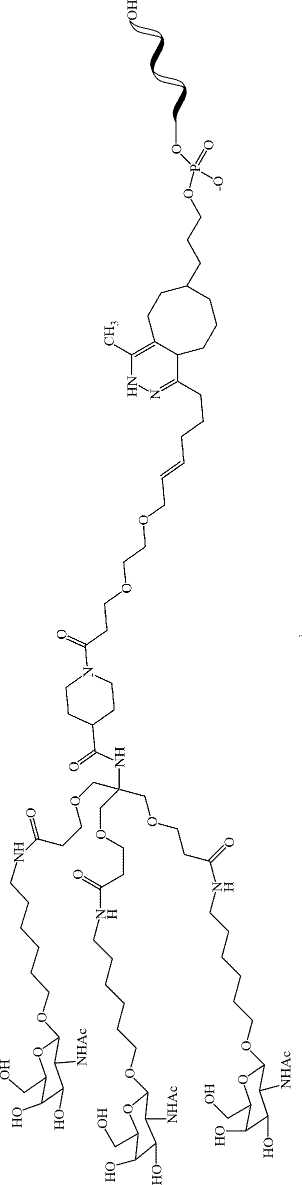

wherein

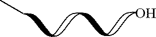

is an oligomer comprising a sense strand and an antisense strand that mediates RNA interference as disclosed herein.

In another aspect, disclosed herein is a pharmaceutical composition comprising an oligomer comprising a sense strand and an antisense strand that mediates RNA interference as disclosed herein, and a compound of Formula (II), (III) and/or (IV), and a pharmaceutically acceptable carrier.

In another aspect, disclosed herein is a pharmaceutical composition comprising an oligomer comprising a sense strand and an antisense strand that mediates RNA interference as disclosed herein, and a compound of Formula (II), (III) and/or (IV), and a lipid of Formula (V) as described herein below.

In some embodiments, X⁷ of the lipid of Formula (V) is S. In some embodiments, R⁷ and R⁸ are each independently selected from the group consisting of methyl, ethyl and isopropyl. In some embodiments, L⁵ and L⁶ are each independently a C₁-C₁₀ alkyl. In some embodiments, L⁵ is C₁₋C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl. In some embodiments, R⁵ and R⁶ are each independently an alkenyl. In some embodiments, R⁶ is alkenyl. In some embodiments, R⁶ is C₂₋C₉ alkenyl. In some embodiments, the alkenyl of R⁵ and R⁶ are each independently comprised of a single double bond. In some embodiments, R⁵ and R⁶ are each alkyl. In some embodiments, R⁵ is a branched alkane. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or -CH((CH₂)ₚCH₃)((CH₂)ₚ₋₁CH₃), wherein p is 4-8. In some embodiments, p is 5 and L⁵ is a C₁₋C₃ alkyl. In some embodiments, p is 6 and L⁵ is a C₃ alkyl. In some embodiments, p is 7. In some embodiments, p is 8 and L⁵ is an C₁₋C₃ alkyl. In some embodiments, R⁵ consists of -CH((CH₂)ₚCH₃)((CH₂)ₚ₋₁CH₃), wherein p is 7 or 8. In some embodiments, R⁴ is ethylene or propylene. In some embodiments, R⁴ is n-propylene or isobutylene. In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, disclosed herein is a pharmaceutical composition comprising an oligomer comprising a sense strand and an antisense strand that mediates RNA interference as disclosed herein, and a compound of Formula (II), (III) and/or (IV), and a lipid of Formula (V) and a pharmaceutically acceptable carrier.

In some embodiments, the pharmaceutical composition is formulated for local or systemic administration.

In some embodiments, the pharmaceutical composition is formulated for intravenous, subcutaneous, pulmonary, intramuscular, intraperitoneal, dermal, or oral administration.

In some embodiments, the pharmaceutical composition further comprises a lipid formulation.

In some embodiments, the pharmaceutical composition further comprises one or more lipids selected from cationic lipids, anionic lipids, sterols, pegylated lipids, or a combination thereof.

In some embodiments, the pharmaceutical composition is substantially free of liposomes. In some embodiments, the pharmaceutical composition contains liposomes.

In some embodiments, the pharmaceutical further comprises a lipid-oligomer nanoparticle comprising a cationic lipid, a cholesterol, a PEG-lipid, and/or a helper lipid.

In some embodiments, the lipid-oligomer nanoparticle has a size less than 100 nm.

In some embodiments, the cationic lipid is a phospholipid.

In some embodiments, the oligomer upregulates, suppresses, reduces, decreases, downregulates or silences the expression of a target gene.

In yet another aspect, disclosed herein is a method for treating or preventing a trinucleotide repeat disease, comprising administering to a subject in need an effective amount of an oligomer disclosed herein.

In some embodiments, the trinucleotide repeat disease is Dentatorubropallidoluysian atrophy, Huntington’s disease, Spinobulbar muscular atrophy or Kennedy disease, Spinocerebellar ataxia type 1, Spinocerebellar ataxia type 2, Spinocerebellar ataxia type 3 or Machado-Joseph disease, Spinocerebellar ataxia type 6, Spinocerebellar ataxia type 7, Spinocerebellar ataxia type 17, Fragile X syndrome, Fragile X-associated tremor/ataxia syndrome, Fragile XE mental retardation, Friedreich’s ataxia, Myotonic dystrophy, Spinocerebellar ataxia Type 8 and/or Spinocerebellar ataxia Type 12.

In some embodiments, the subject is human.

In some embodiments, the method reduces trinucleotide repeat expansion in the subject.

In some embodiments, the effective amount is a dose of from 0.001 to 50.0 mg/kg or 50.0 to 100 mg/kg. In some embodiments, the effective amount is a dose of from 0.001 to 50.0 mg/kg.

In some embodiments, expression of a gene that includes a trinucleotide repeat expansion is reduced for at least 5 days.

In another aspect, disclosed herein is a method for inhibiting expression of a gene that includes a trinucleotide repeat expansion of a gene in a cell, comprising treating the cell with an oligomer as disclosed herein.

In another aspect, disclosed herein is method for inhibiting expression of a gene that includes a trinucleotide repeat expansion in a subject, comprising administering to the mammal an oligomer as disclosed herein.

In another aspect, disclosed herein is a method for treating or preventing a trinucleotide repeat disease, comprising administering to a subject in need an effective amount of a pharmaceutical composition as disclosed herein.

In some embodiments, the trinucleotide repeat disease is Dentatorubropallidoluysian atrophy, Huntington’s disease, Spinobulbar muscular atrophy or Kennedy disease, Spinocerebellar ataxia type 1, Spinocerebellar ataxia type 2, Spinocerebellar ataxia type 3 or Machado-Joseph disease, Spinocerebellar ataxia type 6, Spinocerebellar ataxia type 7, Spinocerebellar ataxia type 17, Fragile X syndrome, Fragile X-associated tremor/ataxia syndrome, Fragile XE mental retardation, Friedreich’s ataxia, Myotonic dystrophy, Spinocerebellar ataxia Type 8 and/or Spinocerebellar ataxia Type 12.

In some embodiments, the subject is human.

In some embodiments, the method reduces expression of a gene that includes a trinucleotide repeat expansion in the subject.

In some embodiments, the effective amount is a dose of from 0.001 to 50.0 mg/kg.

In some embodiments, expression of a gene that includes a trinucleotide repeat expansion is reduced for at least 5 days.

In another aspect, disclosed herein is a method for inhibiting expression of a gene that includes a trinucleotide repeat expansion in a cell, comprising treating the cell with a pharmaceutical composition disclosed herein.

In yet another aspect, disclosed herein is a method for inhibiting expression of a gene that includes a trinucleotide repeat expansion in a subject, comprising administering to the mammal a pharmaceutical composition as disclosed herein.

### Polyglutamine Diseases

The siRNA oligomers of the present disclosure can be used in the treatment of any polyglutamine disease. Exemplary polyglutamine diseases that can be treated by the oligomers of the present disclosure, include, but are not limited to, those discussed below in Table 1.

Dentatorubropallidoluysian Atrophy (DRPLA or Haw-River syndrome) is characterized by cerebellar ataxia (loss of control), epilepsy, myoclonus, choreoathetosis and dementia. Dentatorubropallidoluysian atrophy is a genetic disease caused by the expansion of polyglutamine (polyQ) in atrophin-1 (ATN1) in which the mutation is an expansion of three nucleotide base pairs of cytosine-adenosine-guanine (CAG trinucleotide repeats). Atrophin-1 is a protein expressed in nervous tissue and can be found in the nuclear and cytoplasmic compartments of neuronal cells. It is believed that atrophin-1 acts as a transcriptional co-repressor. The normal CAG repeat size is 6-35 and the disease state CAG repeat size is 49-88. The DRPLA gene is on chromosome 12 (12p13.31). It is likely that mutant DRPLA proteins with expanded polyQ repeats are toxic to neurons and may interact with nuclear protein, interfering with transcription and resulting in neuronal death.

Huntington’s Disease is a progressive neurodegenerative disease caused by CAG trinucleotide repeat expansion leading to mutant huntingtin (HTT) protein. Although the exact function of this protein is unknown, it appears to play an important role in nerve cells (neurons) in the brain and is essential for normal development before birth. Huntingtin is found in many of the body’s tissues, with the highest levels of activity in the brain. Within cells, this protein may be involved in chemical signaling, transporting materials, attaching (binding) to proteins and other structures, and protecting the cell from self-destruction (apoptosis). The severity of the disease is dependent on the number of trinucleotide repeats. The normal CAG repeat size is 6-35 and the disease state CAG repeat size is 36-121. In Huntington’s disease, CAG repeat expansion occurs in the first exon of the HTT gene, which encodes an expanded polyQ stretch in the HTT protein. Accordingly, the severity of the disease depends on the number of polyQ repeats.

Spinobulbar Muscular Atrophy (SBMA or Kennedy disease) is a progressive neuromuscular disorder caused by CAG repeat expansion resulting in atrophy and weakening of the proximal musculature in the limbs. Symptoms include but are not limited to dysarthria, dysphagia, fasciculations, tremors and gait disturbances. (Sperfeld AD, et al., X-linked bulbospinal neuronopathy: Kennedy disease. Arch Neurol. 2002;59(12):1921-6; and Katsuno M, et al, Spinal and bulbar muscular atrophy: ligand-dependent pathogenesis and therapeutic perspectives. J Mol Med (Berl) 2004;82(5):298-307) The normal CAG repeat size is 9-36, and the disease state CAG repeat size is 38-62.

Spinocerebellar Ataxia Type 1 is an autosomal dominant neurodegenerative disorder resulting from CAG repeats within the ATXN1 gene. The normal CAG repeat size is 6-44 and the disease state CAG repeat size is 39-82. Spinocerebellar ataxia type 1 (SCA1) is an autosomal dominant progressive neurodegenerative disorder resulting in a loss of coordination and balance. SCA1 is characterized by neuronal loss in the cerebellum, brain stem and the spinocerebellar tracts (see, for example, Greenfield J. G. (1954). The Spino-Cerebellar Degenerations. Springfield, IL: Blackwell Scientific Publications.; Giunti P., Sweeney M. G., Spadaro M., Jodice C., Novelletto A., Malaspina P., et al. (1994). The trinucleotide repeat expansion on chromosome 6p (SCA1) in autosomal dominant cerebellar ataxias. Brain 117, 645-649. 10.1093/brain/117.4.645; Zoghbi H. Y., Orr H. T. (1995). Spinocerebellar ataxia type 1. Semin. Cell Biol. 6, 29-35. 10.1016/1043-4682(95)90012-8). SCA1 is caused by an expansion of a cytosine-adenine-guanine (CAG) repeat, encoding glutamine, in the gene ATXN1, the function of which is still to be determined.

Spinocerebellar Ataxia Type 2 (SCA2) is an autosomal-dominant neurodegenerative disorder, where SCA2 primarily affects cerebellar Purkinje neurons. The normal CAG repeat size is 15-31 and the disease state CAG repeat size is 36-63. SCA2 patients suffer from a progressive cerebellar syndrome with ataxia of gait and stance, ataxia of limb movements, and dysarthria (see, for example, Schols L, Bauer P, Schmidt T, Schulte T, Riess O. Autosomal dominant cerebellar ataxias: clinical features, genetics, and pathogenesis. Lancet Neurol. 2004; 3; 291-304; Lastres-Becker I, Rub U, Auburger G. Spinocerebellar ataxia 2 (SCA2) Cerebellum. 2008;7:115-24; Filla A, De Michele G, Santoro L, Calabrese O, Castaldo I, Giuffrida S, Restivo D, Serlenga L, Condorelli DF, Bonuccelli U, Scala R, Coppola G, Caruso G, Cocozza S. Spinocerebellar ataxia type 2 in southern Italy: a clinical and molecular study of 30 families. J Neurol. 1999; 246; 467-71). Saccade slowing is a highly characteristic feature that is observed in the majority of SCA2 patients. About half of the patients have vertical or horizontal gaze palsy. Cerebellar oculomotor abnormalities are rarely found in SCA2. Typically, tendon reflexes are absent or decreased. Pyramidal tract signs are present in less than 20% of the patients.

Spinocerebellar Ataxia Type 3 (Machado-Joseph disease) is characterized by progressive cerebellar ataxia and variable findings including a dystonic-rigid syndrome, a parkinsonian syndrome, or a combined syndrome of dystonia and peripheral neuropathy. The normal CAG repeat size is 12-40 and the disease state CAG repeat size is 55-84. Individuals have a 50% chance of inheriting the abnormal CAG trinucleotide expansion in ATXN3.

Spinocerebellar Ataxia Type 6 is a condition characterized by progressive problems with movement. The normal CAG repeat size is 4-18 and the disease state CAG repeat size is 21-33. Patients with this condition experience problems with coordination and balance (ataxia), speech difficulties, involuntary eye movements (nystagmus), and double vision. Over time, individuals with SCA6 may develop loss of coordination in their arms, tremors, and uncontrolled muscle tensing (dystonia). Signs and symptoms of SCA6 typically begin in a patient’s forties or fifties but can appear anytime from childhood to late adulthood. Most people with this disorder require wheelchair assistance by the time they are in their sixties.

Spinocerebellar Ataxia Type 7 is an inherited disease of the central nervous system that leads to impairment of certain nerves in communication to and from the brain, resulting in degeneration of the cerebellum. Visual problems, rather than poor coordination, are generally the earliest signs of the disease. The normal CAG repeat size is 4-35 and the disease state CAG repeat size is 37-306.

Spinocerebellar Ataxia Type 17 is characterized by ataxia, dementia, and involuntary movements, including chorea and dystonia, and psychiatric symptoms, pyramidal signs, and rigidity are common. Onset age ranges are from between 3 to 55 years old. Magnetic resonance imaging (MRI) of the brains of patients shows variable atrophy of the cerebrum, brain stem, and cerebellum. The normal CAG repeat size is 29-42 and the disease state CAG repeat size is 45-63.

Spinocerebellar Ataxia Type 12 (SCA12) is a rare disease caused by CAG trinucleotide repeat expansion in the 5′UTR of the PPP2R2B gene or PP2A-PR550. SCA12 is characterized with the onset of action tremor of the upper extremities in about the subject’s fourth decade, and slowly progressing to include ataxia and other cerebellar and cortical signs. The normal CAG repeat size is 7-28 and the disease state CAG repeat size is 66-78.

TABLE 1

| Disease | Gene | Protein | Normal | Pathogenic |
|---|---|---|---|---|
| Dentatorubropallidoluysian Atrophy (DRPLA) | DRPLA | Atrophin-1 | #PolyQ repeats | #PolyQ repeats |
| Huntington’s Disease (HD) | HD | HTT (Huntingtin) | 6 - 35 | 49 - 88 |
| Spinobulbar Muscular Atrophy or Kennedy Disease (SBMA) | AR | Androgen Receptor | 10 - 35 | > 35 |
| Spinocerebellar Ataxia Type 1 | SCA1 | Ataxin-1 | 9 - 36 | 38 - 62 |
| Spinocerebellar Ataxia Type 2 | SCA2 | Ataxin-2 | 6 - 35 | 49 - 88 |
| Spinocerebellar Ataxia Type 3 or Machado-Joseph disease | SCA3 (MJD1) | Ataxin-3 | 14 - 32 | 33 - 77 |
| Spinocerebellar Ataxia Type 6 | SCA6 | Alpha1A-Voltage-Dependent Calcium Channel Subunit | 12 - 40 | 55 - 86 |
| Spinocerebellar Ataxia Type 7 | SCA7 | Ataxin-7 | 4 - 18 | 21 - 30 |
| Spinocerebellar Ataxia Type 17 | SCA17 | TATA-box binding protein (TBP) | 7 - 17 | 38 - 120 |
| Spinocerebellar ataxia Type 12 | SCA12 | PP2A-PR55beta | 7-28 | 66-78 |

### Other Trinucleotide Repeat Expansion Diseases

The siRNA oligomers of the present disclosure can show high specificity and knockdown activity for other trinucleotide repeat expansions and can be used in the treatment of certain trinucleotide repeat expansion diseases other than those associated with polyglutamine repeat expansion. For example, the trinucleotide repeat expansion can be a repeat of a codon selected from CGG, CCG, GAA, or CTG. Examples of diseases that can be treated by the oligomers of the present disclosure, include, but are not limited to, those discussed below in Table 2.

Fragile X Syndrome results in a range of developmental problems including learning disabilities and cognitive impairment that may involve delayed development of speech and language by age 2. The normal CGG repeat size is 6-53 and the disease state CGG repeat size is > 230.

Fragile X-Associated Tremor/Ataxia (FXTAS) syndrome results in problems with movement and cognition. FXTAS is a late-onset disorder, usually occurring after age 50, and its signs and symptoms worsen with age. Patients have damage in the part of the brain that controls movement and in a type of brain tissue known as white matter. The normal CGG repeat size is 6-53 and the disease state CGG repeat size is 55-200.

Fragile XE Mental Retardation is a genetic disorder that impairs thinking and cognitive function. Some patients with this condition have cognitive function that it is below average. Learning disabilities are common with Fragile XE syndrome. The normal CCG repeat size is 6-35 and the disease state CCG repeat size is > 200.

Friedreich’s Ataxia is a genetic, progressive, neurodegenerative movement disorder, with a typical age of onset between 10 and 15 years. Initial symptoms may include unsteady posture, frequent falling, and progressive difficulty in walking due to impaired ability to coordinate voluntary movements (ataxia). Affected individuals often develop slurred speech (dysarthria), characteristic foot deformities, and an irregular curvature of the spine (scoliosis). FRDA is often associated with cardiomyopathy, a disease of cardiac muscle that may lead to heart failure or irregularities in heart rhythm (cardiac arrhythmias). The normal GAA repeat size is 7-34 and the disease state GAA repeat size is > 100.

Myotonic Dystrophy is characterized by progressive muscle wasting and weakness. Patients with this disorder may have prolonged muscle contractions (myotonia) and may have slurred speech or temporary locking of their jaw. Additional symptoms of myotonic dystrophy include cataracts and abnormalities of the electrical signals that control the heartbeat. In affected men, hormonal changes may lead to early balding and infertility. The normal CTG repeat size is 5-37 and the disease state CTG repeat size is > 50.

Spinocerebellar Ataxia Type 8 is a slowly progressive ataxia with disease onset typically occurring in adulthood. Onset ranges from age one to 73 years. The progression is typically over decades regardless of the age of onset. Common initial symptoms are scanning dysarthria with a characteristic drawn-out slowness of speech and gait instability; life span is typically not shortened. Some individuals present with nystagmus, dysmetric saccades and, rarely, ophthalmoplegia. Tendon reflex hyperreflexivity and extensor plantar responses are present in some severely affected individuals. Life span is typically not shortened. The normal CTG repeat size is 16-37 and the disease state CTG repeat size is 110-250.

TABLE 2

| Disease | Gene | Protein | Codon | Normal | Pathogenic |
|---|---|---|---|---|---|
| Fragile X Syndrome | FMR1 (FRAXA) | FMR-1 Protein (FMRP) | CGG | 6 - 53 | > 230 |
| Fragile X-Associated Tremor/Ataxia Syndrome | FMR1 (FXTAS) | FMR-1 Protein (FMRP) | CGG | 6 - 53 | 55 - 200 |
| Fragile XE mental retardation | FMR2 (FRAXE) | FMR-2 protein | CCG | 6 - 35 | > 200 |
| Friedreich’s ataxia | X25 | Frataxin | GAA | 7 - 34 | > 100 |
| Myotonic dystrophy (MD) | DMPK | Dystrophy Protein Kinase (DMPK) | CTG | 5 - 37 | > 50 |
| Spinocerebellar ataxia Type 8 | SCA8 | Ataxin-8 | CTG | 16 - 37 | 110 - 250 |

In some embodiments, this disclosure provides active agents for efficient gene silencing and knockdown of polyQ or other repeat expansion with reduced off target effects.

In certain aspects, this disclosure provides therapeutics for polyQ and other trinucleotide repeat-related diseases as described above.

### UNA Monomers and Oligomers

In some embodiments, the oligomer comprising a sense strand and an antisense strand that mediates RNA interference as disclosed herein comprises one or more UNA monomers that are small organic molecules based on a propane-1,2,3-tri-yl-trisoxy structure as shown below:

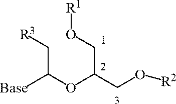

wherein R¹ and R² can be H or R¹ and R² can be phosphodiester linkages (the O to which R¹ or R² is attached would be part of the phosphodiester linkage), Base can be a natural or modified nucleobase, and R³ is a functional group selected from OR⁴, SR⁴, NR⁴R⁴, —NH(C═O)R4, morpholino, morpholin-1-yl, piperazin-1-yl, or 4-alkanoyl-piperazin-1-yl, where R⁴ is the same or different for each occurrence, and can be H, alkyl, a cholesterol, a lipid molecule, a polyamine, an amino acid, or a polypeptide. Examples of a nucleobase include uracil, thymine, cytosine, 5-methylcytosine, adenine, guanine, and inosine. Further examples include those natural and non-natural nucleobase analogues and UNA monomers found in U.S. 2018/0362985, the contents of which are incorporated herein by reference.

In general, because the UNA monomers are not nucleotides, they can exhibit at least four forms in an oligomer. First, a UNA monomer can be an internal monomer in an oligomer, where the UNA monomer is flanked by other monomers on both sides. In this form, the UNA monomer can participate in base pairing when the oligomer is a duplex, for example, and there are other monomers with nucleobases in the duplex.

Second, a UNA monomer can be a monomer in an overhang of an oligomer duplex, where the UNA monomer is flanked by other monomers on both sides. In this form, the UNA monomer does not participate in base pairing. Because the UNA monomers are flexible organic structures, unlike nucleotides, the overhang containing a UNA monomer will be a flexible terminator for the oligomer.

Third, A UNA monomer can be a terminal monomer in an overhang of an oligomer, where the UNA monomer is attached to only one monomer at either the propane-1-yl position or the propane-3-yl position. In this form, the UNA monomer does not participate in base pairing.

Fourth, because the UNA monomers are flexible organic structures, unlike nucleotides, the overhang containing a UNA monomer can be a flexible terminator for the oligomer and assume different conformations. Thus, UNA oligomers having a terminal UNA monomer are significantly different in structure from conventional nucleic acid agents, such as siRNAs. For example, siRNAs may require that terminal monomers or overhangs in a duplex be stabilized. In contrast, the conformability of a terminal UNA monomer can provide UNA oligomers with different properties.

Oligomeric compounds comprising one or more UNA monomers (UNA oligomers) can be prepared by automated oligonucleotide synthesis as known to a person skilled in the art. The incorporation of the UNA monomers of the invention into the oligonucleotides of the invention follows standard methods for oligonucleotide synthesis, work-up, purification and isolation (see, for example, F. Eckstein, Oligonucleotides and Analogues, IRL Press, Oxford University Press, 1991) with modifications as published (Johannsen, M. W. et al., Org. Biomol. Chem.,2011, 9, 243).

A UNA oligomer of this disclosure is a synthetic chain molecule. A UNA oligomer of this disclosure is not a nucleic acid, nor is it an oligonucleotide.

In some embodiments, a UNA monomer can be UNA-A (designated Ã), UNA-U (designated Ũ), UNA-C (designated C), and UNA-G (designated G).

Other designations that may be used herein include mA, mG, mC, and mU, which refer to 2′-O-Methyl modified ribonucleotides. In some instances, the 2′-O-methyl modified ribonucleotides may also be designated by a lower case a, g, c, or u.

Further designations that may be used herein include T and dT, which refers to a 2′-deoxy T nucleotide.

As used herein the designations rA, rG, rC, and rU include a natural or modified ribonucleotide, including modifications on the ribose sugar portion, the purine or pyrimidine base portion, or both. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′-O-dimethyl-adenosine, N⁶,N⁶,2′-O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically-modified nucleotides include N¹-alkylguanosines, N²-alkylguanosines, thienoguanosines, 7-deazaguanosines, 8-oxoguanosines, 8-bromoguanosines, O6-alkylguanosines, xanthosines, inosines, and N¹-alkylinosines.

Examples of modified or chemically-modified nucleotides include N¹-methylguanosine, N²-methylguanosine, thienoguanosine, 7-deazaguanosine, 8-oxoguanosine, 8-bromoguanosine, O6-methylguanosine, xanthosine, inosine, and N¹-methylinosine.

Examples of modified or chemically-modified nucleotides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “N1MPU”), N¹-ethylpseudouridine, N¹-propylpseudouridine, N¹-cyclopropylpseudouridine, N¹-phenylpseudouridine, N1-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N⁶-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethy-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′-O-methyluridine, 3′-O-dimethyluridine, and 2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenosine, 2-methoxy-adenosine, alpha-thio-adenosine, 2′-O-methyl-adenosine, N⁶,2′-O-dimethyl-adenosine, N⁶,N⁶,2′-O-trimethyl-adenosine, 2′-O-dimethyl-adenosine, 2′-O-ribosyladenosine, 2-amino-N⁶-methyl-purine, 1-thio-adenosine, 2′-fluoro-ara-adenosine, 2′-fluoro-adenosine, 2′-OH-ara-adenosine, and N⁶-(19-amino-pentaoxanonadecyl)-adenosine.

Examples of modified or chemically-modified nucleotides include N¹-alkylguanosines, N²-alkylguanosines, thienoguanosines, 7-deazaguanosines, 8-oxoguanosines, 8-bromoguanosines, O⁶-alkylguanosines, xanthosines, inosines, and N¹-alkylinosines.

Examples of modified or chemically-modified nucleotides include N¹-methylguanosine, N²-methylguanosine, thienoguanosine, 7-deazaguanosine, 8-oxoguanosine, 8-bromoguanosine, O⁶-methylguanosine, xanthosine, inosine, and N¹-methylinosine.

Examples of modified or chemically-modified nucleotides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹phenylalkylpseudouridines, N¹aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Other examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “NIMPU”), N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′- inverted thymidine.

Examples of modified and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an embodiment, the modified monomer is a locked nucleic acid nucleotide (LNA).

Examples of modified and chemically-modified nucleotide monomers include 2′,4′-constrained 2′-O-methoxy ethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N⁶-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′—OH group of a nucleotide with a 2′—R, a 2′—OR, a 2′-halogen, a 2′—SR, or a 2′-amino, where R can be H, alkyl, alkenyl, or alkynyl.

Some further examples of modified nucleotides are given in Saenger, Principles of Nucleic Acid Structure, Springer-Verlag, 1984.

Any of the example base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine.

A UNA oligomer may comprise two strands that together provide a duplex. The duplex may be composed of a first strand, which may also be referred to as a passenger strand or sense strand, and a second strand, which may also be referred to as a guide strand or antisense strand.

In some aspects, a UNA oligomer of this disclosure can have any number of phosphorothioate intermonomer linkages in any position in any strand, or in both strands of a duplex structure.

Examples of UNA oligomers of this disclosure include duplex pairs, which are in general complementary. Thus, for example, SEQ ID NO: 1′ can represent a first strand of a duplex and SEQ ID NO: 2′ can represent a second strand of the duplex, which is complementary to the first strand, wherein the symbol “N” in the first strand can represent any nucleotide that is complementary to the monomer in the corresponding position in the second strand and the symbol “X” in a strand or oligomer represents a UNA monomer. Example UNA oligomers of this disclosure are shown with 2-monomer length overhangs, although overhangs of from 1 to 8 monomers, or longer, can be used.

For example, the UNA oligomer

SEQ ID NO:1′
5′-X N N▪N▪N▪N▪N▪N▪N▪N▪N▪N▪N▪N▪N▪N▪X▪X-3′

3′-X▪X▪N▪N▪N▪N▪N▪N▪N▪N▪N▪N▪N▪X▪X▪X▪X▪X▪X▪X▪N-5′
SEQ ID NO:2′

has a UNA monomer 5′-end on the first strand (depicted on top), a UNA monomer 3′-end on the first strand, a UNA monomer 3′-end on the second strand (depicted on bottom), and a nucleotide 5′-end on the second strand.

In some embodiments, a UNA oligomer of this disclosure can have one or more UNA monomers at the 5′-end of the first strand, and/or one or more UNA monomers at the 3′-end of the first strand. In further embodiments, a UNA oligomer of this disclosure can have one or more UNA monomers at the 3′-end of the second strand. In certain embodiments, a duplex UNA oligomer of this disclosure can have one or more UNA monomers at the 5′-end of the first strand, one or more UNA monomers at the 3′-end of the first strand, and one or more UNA monomers at the 3′-end of the second strand. In some embodiments, the oligomer comprises a UNA monomer at the first position at the 5′-end of the sense strand. In some embodiments, the oligomer comprises a UNA monomer at the first position at the 5′-end of the sense strand, and a UNA monomer at the ninth position from the 5′-end of the antisense strand. In some embodiments, the oligomer comprises a UNA monomer at the first position at the 5′-end of the sense strand, and a UNA monomer at the tenth position from the 5′-end of the antisense strand. In some embodiments, the oligomer comprises a UNA monomer at the first position at the 5′-end of the sense strand, and a UNA monomer at one or both of the ninth and tenth positions from the 5′-end of the antisense strand. In some embodiments, the oligomer comprises a UNA monomer at the first position at the 5′-end of the sense strand, and a UNA monomer at one or both of the last two positions from the 3′-end of the sense strand. In some embodiments, the oligomer comprises a UNA monomer at the first position at the 5′-end of the sense strand, and a UNA monomer at one or more of the last two positions from the 3′-end of the antisense strand. In some embodiments, the oligomer comprises a UNA monomer at one or more of the last two positions from the 3′-end of the sense strand, and a UNA monomer at one or more of the last two positions from the 3′-end of the antisense strand.

A UNA oligomer of this disclosure may have a first strand (e.g., sense strand) and a second strand (e.g., antisense strand), each of the strands independently being 19-29 monomers in length. In certain embodiments, a UNA oligomer of this disclosure may have a first strand that is 19-23 monomers in length. In certain embodiments, a UNA oligomer of this disclosure may have a duplex region that is 19-21 monomers in length. In further embodiments, a UNA oligomer of this disclosure may have a second strand that is 19-23 monomers in length. In certain embodiments, a UNA oligomer of this disclosure may have a first strand that is 19 monomers in length, and a second strand that is 19 monomers in length. In certain embodiments, a UNA oligomer of this disclosure may have a first strand that is 19 monomers in length, and a second strand that is 21 monomers in length. In certain embodiments, a UNA oligomer of this disclosure may have a first strand that is 20 monomers in length, and a second strand that is 21 monomers in length. In certain embodiments, a UNA oligomer of this disclosure may have a first strand that is 21 monomers in length, and a second strand that is 21 monomers in length. In certain embodiments, a UNA oligomer of this disclosure may have a first strand that is 22 monomers in length, and a second strand that is 21 monomers in length.

A UNA oligomer of this disclosure for inhibiting gene expression can have a first strand and a second strand, each of the strands being 19-29 monomers in length. The monomers can be UNA monomers and nucleic acid monomers. The oligomer can have a duplex structure of from 14 to 29 monomers in length. The UNA oligomer can be targeted to a target gene and can exhibit reduced off-target effects as compared to a conventional siRNA. In some embodiments, a UNA oligomer of this disclosure can have a first strand and a second strand, each of the strands being 19-23 monomers in length.

In another aspect, the UNA oligomer may have a blunt end, or may have one or more overhangs. In some embodiments, the first and second strands may be connected with a connecting oligomer in between the strands and form a duplex region with a connecting loop at one end.

In certain embodiments, an overhang can be one or two monomers in length.

A UNA oligomer can mediate cleavage of a target nucleic acid in a cell. In some processes, the second strand of the UNA oligomer, at least a portion of which can be complementary to the target nucleic acid, can act as a guide strand (antisense strand) that can hybridize to the target nucleic acid.

The second strand can be incorporated into an RNA Induced Silencing Complex (RISC).

A UNA oligomer of this disclosure may comprise naturally-occurring nucleic acid nucleotides, and modifications thereof that are compatible with gene silencing activity.

In some aspects, a UNA oligomer is a double stranded construct molecule that is able to inhibit gene expression.

As used herein, the term strand refers to a single, contiguous chain of monomers, the chain having any number of internal monomers and two end monomers, where each end monomer is attached to one internal monomer on one side and is not attached to a monomer on the other side, so that it ends the chain.

The monomers of a UNA oligomer may be attached via phosphodiester linkages, phosphorothioate linkages, gapped linkages, and other variations.

In some embodiments, a UNA oligomer can include mismatches in complementarity between the first and second strands. In other embodiments, a UNA oligomer may have 1, or 2, or 3 mismatches. The mismatches may occur at any position in the duplex region.

The target of a UNA oligomer can be a target nucleic acid of a target gene.

A UNA oligomer may have one or two overhangs outside the duplex region. The overhangs can be an unpaired portion at the end of the first strand or second strand. The lengths of the overhang portions of the first and second strands can be the same or different.

A UNA oligomer may have at least one blunt end. A blunt end does not have an overhang portion, and the duplex region at a blunt end terminates at the same position for both the first and second strands.

A UNA oligomer can be RNA-induced silencing complex (RISC) length, which means that it has a duplex length of less than 25 base pairs.

In certain embodiments, a UNA oligomer can be a single strand that folds upon itself and hybridizes to itself to form a double stranded region having a connecting loop.

In some embodiments, disclosed herein is a UNA oligomer having reduced off-target effects that can have a UNA monomer at the first position at the 5′-end of the first strand, also called the passenger strand, and one or both of the last two positions from the 3′-end of the first strand, as well as one or both of the last two positions from the 3′-end of the second strand, also called the guide strand. In some embodiments, a UNA oligomer having reduced off-target effects can have a UNA monomer at the first position at the 5′-end of the first strand, and one or both of the last two positions from the 3′-end of the first strand. In some embodiments, a UNA oligomer having reduced off-target effects can have a UNA monomer at the first position at the 5′-end of the first strand, also called the passenger strand, and one or more of the last two positions from the 3′-end of the second strand. In some embodiments, a UNA oligomer having reduced off-target effects can have a UNA monomer at the first position at the 5′-end of the first strand. In some embodiments, a UNA oligomer having reduced off-target effects can have a UNA monomer at one or more of the last two positions from the 3′-end of the first strand, as well as one or more of the last two positions from the 3′-end of the second strand. In some embodiments, in addition to having one or more UNA monomers at any of the positions described above, a UNA oligomer having reduced off-target effects can have a UNA monomer in the seed region at any one or more of positions 2-12 from the 5′-end of the second strand. In some embodiments, a UNA oligomer having reduced off-target effects can have a UNA monomer at the first position at the 5′-end of the sense strand. In some embodiments, a UNA oligomer having reduced off-target effects can have a UNA monomer at the first position at the 5′-end of the sense strand, and a UNA monomer at the ninth position from the 5′-end of the antisense strand. In some embodiments, a UNA oligomer having reduced off-target effects can have a UNA monomer at the first position at the 5′-end of the sense strand, and a UNA monomer at the tenth position from the 5′-end of the antisense strand. In some embodiments, a UNA oligomer having reduced off-target effects can have a UNA monomer at the first position at the 5′-end of the sense strand, and a UNA monomer at one or both of the ninth and tenth positions from the 5′-end of the antisense strand. In some embodiments, a UNA oligomer having reduced off-target effects can have a UNA monomer at the first position at the 5′-end of the sense strand, and a UNA monomer at one or both of the last two positions from the 3′-end of the sense strand. In some embodiments, a UNA oligomer having reduced off-target effects can have a UNA monomer at the first position at the 5′-end of the sense strand, and a UNA monomer at one or more of the last two positions from the 3′-end of the antisense strand. In some embodiments, a UNA oligomer having reduced off-target effects can have a UNA monomer at one or more of the last two positions from the 3′-end of the sense strand, and a UNA monomer at one or more of the last two positions from the 3′-end of the antisense strand.

In some embodiments, disclosed herein is a UNA oligomer having reduced off-target effects comprising a sense strand and an antisense strand as presented below in Table 3.

TABLE 3

| SEQ ID NO. | Oligo Name | Sense strand (5′ → 3′) | SEQ ID NO. | Antisense strand (5′ → 3′) |
|---|---|---|---|---|
| 1 | NTC | UrArGrCrGrArCrUrArArArCrArCrArUrCrGrCTT | 3 | rGrCrGrArUrGrUrGrUrUrUrArGrUrCrGrCrUrATT |
| 2 | REP | CrArGrCrArGrCrArGrCrArGrCrArGrCrArGrCTT | 4 | rGrCrUrGrCrUrGrCrUrGrCrUrGrCrUrGrCrUrGTT |
| 2 | REPU3 | CrArGrCrArGrCrArGrCrArGrCrArGrCrArGrCTT | 5 | rGrCUrGrCrUrGrCrArGrCrUrGrCrUrGrCrUrGTT |
| 2 | REPU5 | CrArGrCrArGrCrArGrCrArGrCrArGrCrArGrCTT | 6 | rGrCrUrGCrUrGrCrArGrCrUrGrCrUrGrCrUrGTT |
| 2 | REPU7 | CrArGrCrArGrCrArGrCrArGrCrArGrCrArGrCTT | 7 | rGrCrUrGrCrUGrCrArGrCrUrGrCrUrGrCrUrGTT |
| 2 | REPU9 | CrArGrCrArGrCrArGrCrArGrCrArGrCrArGrCTT | 8 | rGrCrUrGrCrUrGrCArGrCrUrGrCrUrGrCrUrGTT |
| 2 | REPU10 | CrArGrCrArGrCrArGrCrArGrCrArGrCrArGrCTT | 9 | rGrCrUrGrCrUrGrCrAGrCrUrGrCrUrGrCrUrGTT |
| 2 | REPU910 | CrArGrCrArGrCrArGrCrArGrCrArGrCrArGrCTT | 10 | rGrCrUrGrCrUrGrCAGrCrUrGrCrUrGrCrUrGTT |
| 2 | REPU1011 | CrArGrCrArGrCrArGrCrArGrCrArGrCrArGrCTT | 11 | rGrCrUrGrCrUrGrCrAGCrUrGrCrUrGrCrUrGTT |
| 2 | REPU11 | CrArGrCrArGrCrArGrCrArGrCrArGrCrArGrCTT | 12 | rGrCrUrGrCrUrGrCrArGCrUrGrCrUrGrCrUrGTT |
| 2 | REPU13 | CrArGrCrArGrCrArGrCrArGrCrArGrCrArGrCTT | 13 | rGrCrUrGrCrUrGrCrArGrCrUGrCrUrGrCrUrGTT |
| 2 | REPU15 | CrArGrCrArGrCrArGrCrArGrCrArGrCrArGrCTT | 14 | rGrCrUrGrCrUrGrCrArGrCrUrGrCUrGrCrUrGTT |

N: DNA, rN: RNA, N: UNA

### Lipid-Based Formulations

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., siRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 Jun; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane, release at the cytoplasm (for RNAs), and so on. Successful nucleic acid-based therapy thus depends upon the ability of the vector to deliver the nucleic acids to the target sites inside of the cells in order to obtain sufficient levels of a desired activity such as expression of a gene or interference of translation.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including siRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (see, for example, Int. J. Nanomedicine. 2014; 9: 1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid, which can complex to and associate with the negatively charged backbone of the nucleic acid core. Ionizable cationic lipids with apparent pKa values below about 7 have the benefit of providing a cationic lipid for complexing with the nucleic acid’s negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following intravenous (i.v.) administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation.

### Lipid-siRNA (UNA Oligomer) Formulations

An siRNA or UNA oligomer as disclosed herein or a pharmaceutically acceptable salt thereof can be incorporated into a lipid formulation (i.e., a lipid-based delivery vehicle).

In the context of the present disclosure, a lipid-based delivery vehicle typically serves to transport a desired UNA oligomer to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing a UNA oligomer of the present disclosure. In some embodiments, the lipid-based delivery vehicle comprises a nanoparticle or a bilayer of lipid molecules and a UNA oligomer of the present disclosure. In some embodiments, the lipid bilayer preferably further comprises a neutral lipid or a polymer. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic UNA oligomer molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the UNA oligomer is fully encapsulated within the lipid portion of the lipid formulation such that the UNA oligomer in the lipid formulation is resistant in aqueous solution to nuclease degradation. In other embodiments, the lipid formulations described herein are substantially non-toxic to mammals such as humans.

The lipid formulations of the disclosure also typically have a total lipid:UNA oligomer ratio (mass/mass ratio) of from about 1:1 to about 100:1, from about 1:1 to about 50:1, from about 2:1 to about 45:1, from about 3:1 to about 40:1, from about 5:1 to about 38:1, or from about 10:1 to about 40:1, or from about 15:1 to about 35:1, or from about 20:1 to about 40:1; or from about 25:1 to about 35:1; or from about 27:1 to about 32:1; or from about 28:1 to about 32:1; or from about 29:1 to about 31:1. In some preferred embodiments, the total lipid:UNA oligomer ratio (mass/mass ratio) is from about 25:1 to about 35:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise a UNA oligomer, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol.

In the nucleic acid-lipid formulations, the UNA oligomer may be fully encapsulated within the lipid portion of the formulation, thereby protecting the nucleic acid from nuclease degradation. In preferred embodiments, a lipid formulation comprising a UNA oligomer is fully encapsulated within the lipid portion of the lipid formulation, thereby protecting the nucleic acid from nuclease degradation. In certain instances, the UNA oligomer in the lipid formulation is not substantially degraded after exposure of the particle to a nuclease at 37° C. for at least 20, 30, 45, or 60 minutes. In certain other instances, the UNA oligomer in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the UNA oligomer is complexed with the lipid portion of the formulation. One of the benefits of the formulations of the present disclosure is that the nucleic acid-lipid compositions are substantially non-toxic to mammals such as humans.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E = (I₀ - I)/I₀, where/and I₀ refers to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of UNA oligomer-liposomes. In some embodiments, the nucleic acid-lipid composition comprises a plurality of UNA oligomer-cationic liposomes. In some embodiments, the nucleic acid-lipid composition comprises a plurality of UNA oligomer-lipid nanoparticles.

In some embodiments, the lipid formulations comprise UNA oligomer that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the UNA oligomer encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, the UNA oligomers described herein are lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) a UNA oligomer of the present disclosure,
(b) a cationic lipid,
(c) an aggregation reducing agent (such as polyethylene glycol (PEG) lipid or PEG- modified lipid),
(d) optionally a non-cationic lipid (such as a neutral lipid), and
(e) optionally, a sterol.

In one some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. Exemplary cationic lipids (including ionizable cationic lipids), helper lipids (e.g., neutral lipids), sterols, and ligand-containing lipids (e.g., PEG-lipids) are described hereinbelow.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA). Cationic lipids, such as DOTAP, (1,2-dioleoyl-3- trimethylammonium-propane) and DOTMA (N-[1-(2,3-dioleoyloxy)propyl]-N,N,N-trimethyl- ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N- dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P-(N-(N′,N′-dimethylaminoethane)- carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Pat. Publication Nos. 2011/0256175, 2012/0128760, and 2012/0027803; U.S. Pat. Nos. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl- N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In some embodiments, the lipid formulation comprises the cationic lipid with Formula (I) as described in WO 2018/078053. In this context, the disclosure of WO 2018/078053 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula (V)

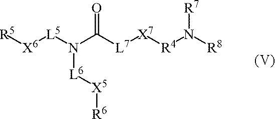

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂₋C₃₁ alkynyl and cholesteryl; L⁵ and L⁶ are each independently selected from the group consisting of a linear C₁₋C₂₀ alkyl and C₂₋C₂₀ alkenyl; X⁵ is —C(O)O—, whereby —C(O)OR⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁₋C₆ alkyl; and R⁷ and R⁸ are each independently selected from the group consisting of a hydrogen and a linear or branched C₁₋C₆ alkyl.

In some embodiments, X⁷ is S.

In some embodiments, X⁵ is —C(O)O—, whereby —C(O)O—R⁶ is formed and X⁶ is —C(O)O— whereby —C(O)O—R⁵ is formed.

In some embodiments, R⁷ and R⁸ are each independently selected from the group consisting of methyl, ethyl and isopropyl.

In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl.

In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁₋C₃ alkyl. In some embodiments, p is 6 and L⁵ is a C₃ alkyl. In some embodiments, p is 7. In some embodiments, p is 8 and L⁵ is a C₁₋C₃ alkyl. In some embodiments, R⁵ consists of -CH((CH₂)ₚCH₃)((CH₂)ₚ₋₁CH₃), wherein p is 7 or 8.

In some embodiments, R⁴ is ethylene or propylene. In some embodiments, R⁴ is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X⁵ is —C(O)O—, whereby —C(O)O—R⁶ is formed, X⁶ is —C(O)O— whereby —C(O)O—R⁵ is formed, L⁵ and L⁶ are each independently a linear C₃₋C₇ alkyl, L⁷ is absent, R⁵ is —CH((CH₂)ₚCH₃)₂, and R⁶ is C₇₋C₁₂ alkenyl. In some further embodiments, p is 6 and R⁶ is C₉ alkenyl.

In some embodiments, the lipid formulation comprises an ionizable cationic lipid selected from the group consisting of

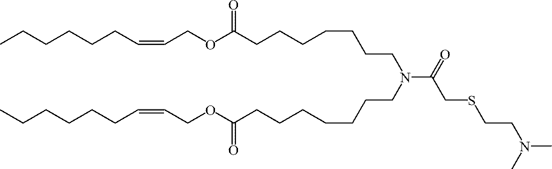 [SMILES: CCCCCC/C=C\COC(=O)CCCCCCCN(CCCCCCCC(=O)OC/C=C\CCCCCC)C(=O)CSCCN(C)C]

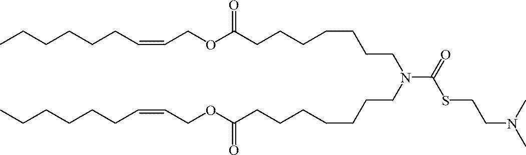 [SMILES: CCCCCC/C=C\COC(=O)CCCCCCCN(CCCCCCCC(=O)OC/C=C\CCCCCC)C(=O)SCCN(C)C]

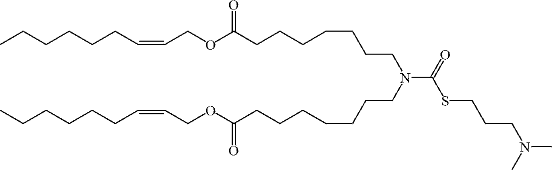 [SMILES: CCCCCC/C=C\COC(=O)CCCCCCCN(CCCCCCCC(=O)OC/C=C\CCCCCC)C(=O)SCCCN(C)C]

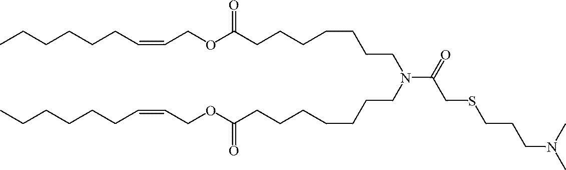 [SMILES: CCCCCC/C=C\COC(=O)CCCCCCCN(CCCCCCCC(=O)OC/C=C\CCCCCC)C(=O)CSCCCN(C)C]

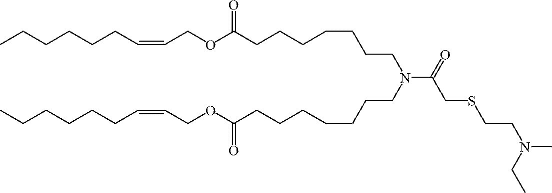 [SMILES: CCCCCC/C=C\COC(=O)CCCCCCCN(CCCCCCCC(=O)OC/C=C\CCCCCC)C(=O)CSCCN(C)CC]

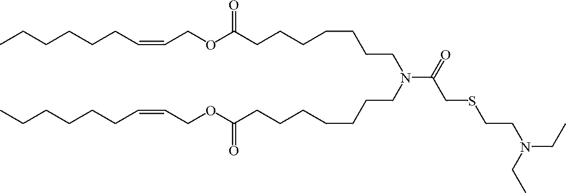 [SMILES: CCCCCC/C=C\COC(=O)CCCCCCCN(CCCCCCCC(=O)OC/C=C\CCCCCC)C(=O)CSCCN(CC)CC]

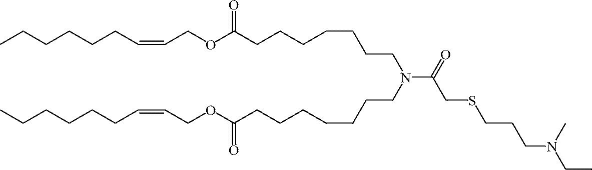 [SMILES: CCCCCC/C=C\COC(=O)CCCCCCCN(CCCCCCCC(=O)OC/C=C\CCCCCC)C(=O)CSCCCN(C)CC]

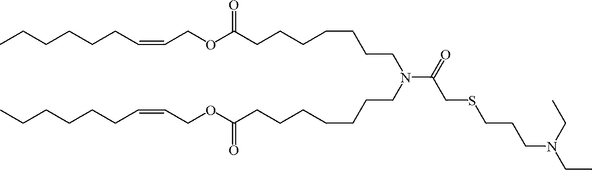 [SMILES: CCCCCC/C=C\COC(=O)CCCCCCCN(CCCCCCCC(=O)OC/C=C\CCCCCC)C(=O)CSCCCN(CC)CC]

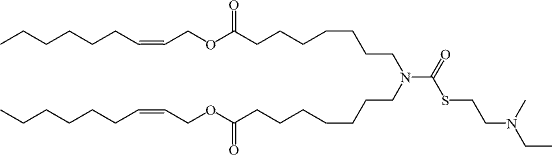 [SMILES: CCCCCC/C=C\COC(=O)CCCCCCCN(CCCCCCCC(=O)OC/C=C\CCCCCC)C(=O)SCCN(C)CC]

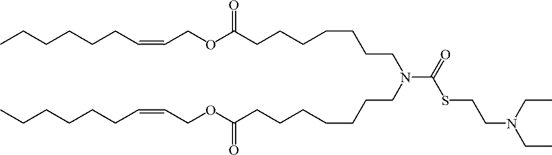 [SMILES: CCCCCC/C=C\COC(=O)CCCCCCCN(CCCCCCCC(=O)OC/C=C\CCCCCC)C(=O)SCCN(CC)CC]

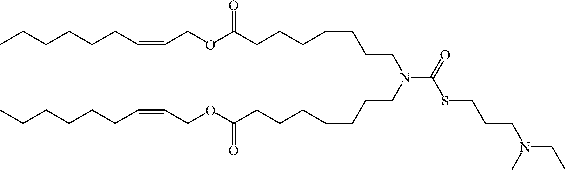 [SMILES: CCCCCC/C=C\COC(=O)CCCCCCCN(CCCCCCCC(=O)OC/C=C\CCCCCC)C(=O)SCCCN(C)CC]

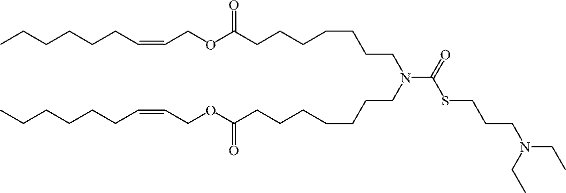 [SMILES: CCCCCC/C=C\COC(=O)CCCCCCCN(CCCCCCCC(=O)OC/C=C\CCCCCC)C(=O)SCCCN(CC)CC]

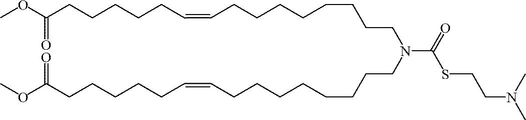

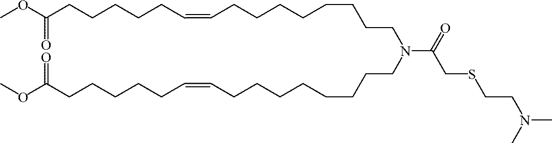

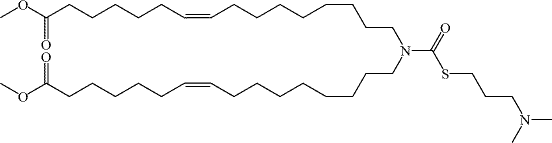

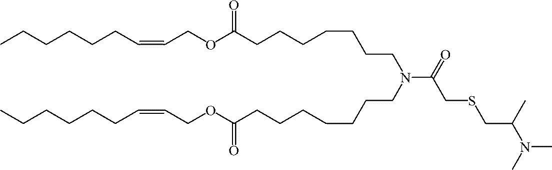 [SMILES: CCCCCC/C=C\COC(=O)CCCCCCCN(CCCCCCCC(=O)OC/C=C\CCCCCC)C(=O)CSCC(C)N(C)C]

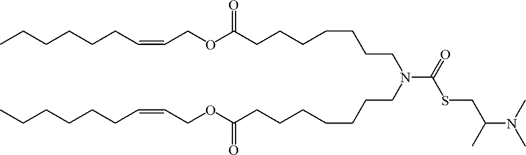 [SMILES: CCCCCC/C=C\COC(=O)CCCCCCCN(CCCCCCCC(=O)OC/C=C\CCCCCC)C(=O)SCC(C)N(C)C]

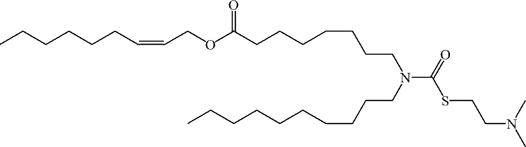 [SMILES: CCCCCC/C=C\COC(=O)CCCCCCCN(CCCCCCCCCC)C(=O)SCCN(C)C]

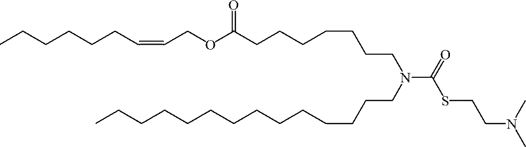 [SMILES: CCCCCC/C=C\COC(=O)CCCCCCCN(CCCCCCCCCCCCCC)C(=O)SCCN(C)C]

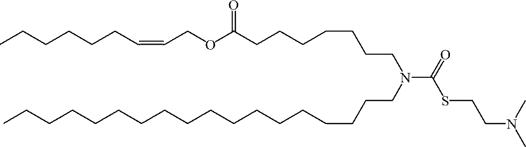 [SMILES: CCCCCC/C=C\COC(=O)CCCCCCCN(CCCCCCCCCCCCCCCCCC)C(=O)SCCN(C)C]

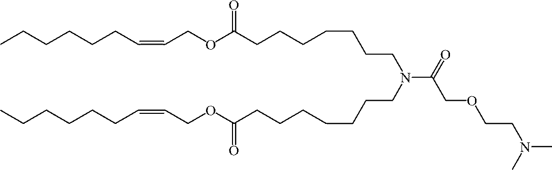 [SMILES: CCCCCC/C=C\COC(=O)CCCCCCCN(CCCCCCCC(=O)OC/C=C\CCCCCC)C(=O)COCCN(C)C]

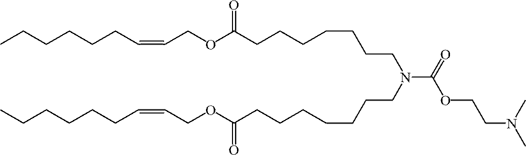 [SMILES: CCCCCC/C=C\COC(=O)CCCCCCCN(CCCCCCCC(=O)OC/C=C\CCCCCC)C(=O)OCCN(C)C]

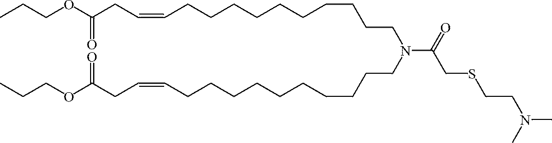 [SMILES: CCCOC(=O)C/C=C\CCCCCCCCCCN(CCCCCCCCCC/C=C\CC(=O)OCCC)C(=O)CSCCN(C)C]

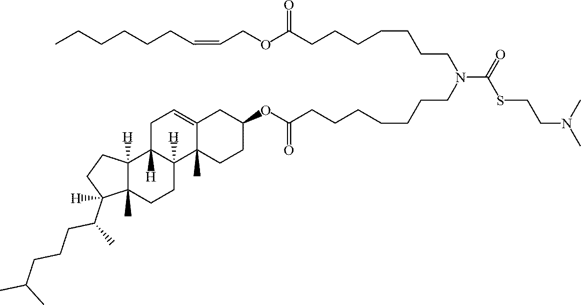

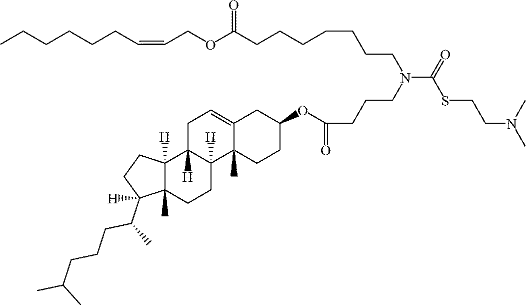

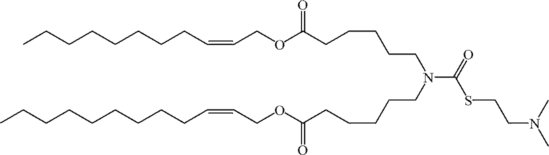 [SMILES: CCCCCCCC/C=C\COC(=O)CCCCCN(CCCCCC(=O)OC/C=C\CCCCCCCCC)C(=O)SCCN(C)C]

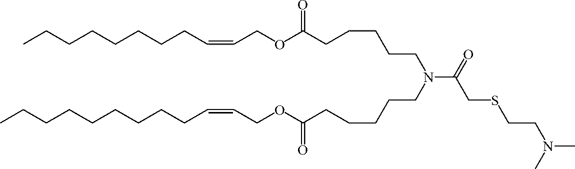 [SMILES: CCCCCCCC/C=C\COC(=O)CCCCCN(CCCCCC(=O)OC/C=C\CCCCCCCCC)C(=O)CSCCN(C)C]

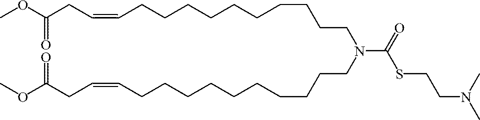

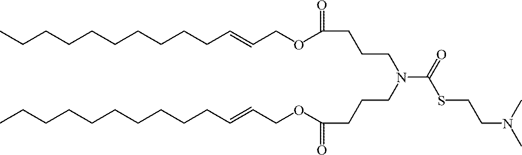 [SMILES: CCCCCCCCCC/C=C/COC(=O)CCCN(CCCC(=O)OC/C=C/CCCCCCCCCC)C(=O)SCCN(C)C]

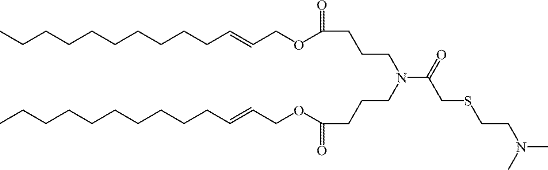 [SMILES: CCCCCCCCCC/C=C/COC(=O)CCCN(CCCC(=O)OC/C=C/CCCCCCCCCC)C(=O)CSCCN(C)C]

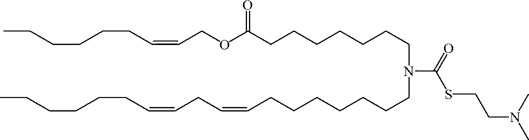 [SMILES: CCCCCC/C=C\C/C=C\CCCCCCCN(CCCCCCCC(=O)OC/C=C\CCCCCC)C(=O)SCCN(C)C]

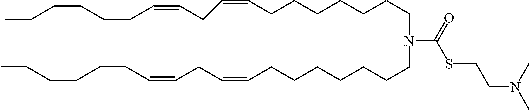 [SMILES: CCCCCC/C=C\C/C=C\CCCCCCCN(CCCCCCC/C=C\C/C=C\CCCCCC)C(=O)SCCN(C)C]

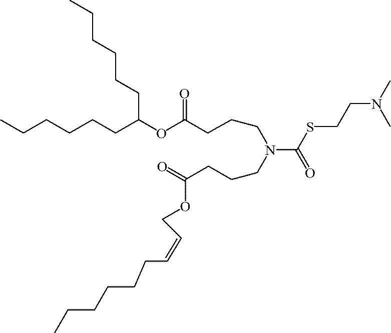 [SMILES: CCCCCC/C=C\COC(=O)CCCN(CCCC(=O)OC(CCCCCC)CCCCCC)C(=O)SCCN(C)C]

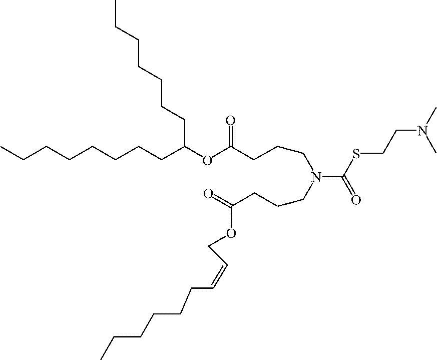 [SMILES: CCCCCC/C=C\COC(=O)CCCN(CCCC(=O)OC(CCCCCCC)CCCCCCCC)C(=O)SCCN(C)C]

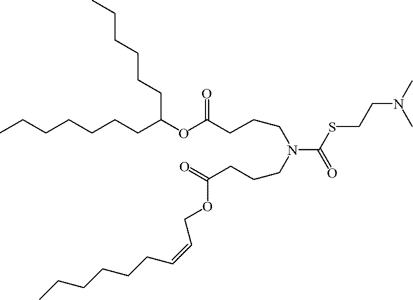 [SMILES: CCCCCC/C=C\COC(=O)CCCN(CCCC(=O)OC(CCCCCC)CCCCCCC)C(=O)SCCN(C)C]

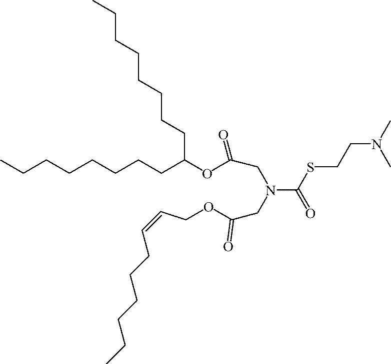 [SMILES: CCCCCC/C=C\COC(=O)CN(CC(=O)OC(CCCCCCCC)CCCCCCCC)C(=O)SCCN(C)C]

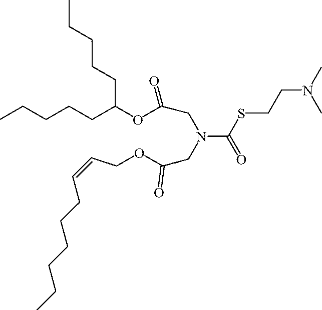 [SMILES: CCCCCC/C=C\COC(=O)CN(CC(=O)OC(CCCCC)CCCCC)C(=O)SCCN(C)C]

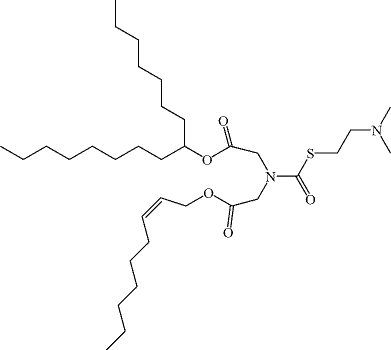 [SMILES: CCCCCC/C=C\COC(=O)CN(CC(=O)OC(CCCCCCC)CCCCCCCC)C(=O)SCCN(C)C]

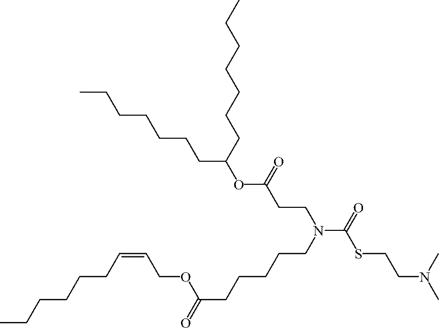 [SMILES: CCCCCC/C=C\COC(=O)CCCCCN(CCC(=O)OC(CCCCCCC)CCCCCCC)C(=O)SCCN(C)C]

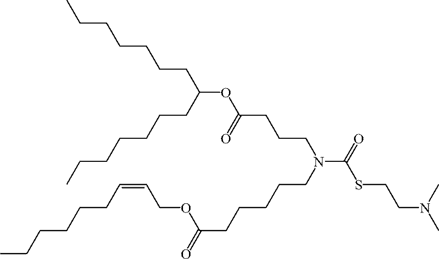 [SMILES: CCCCCC/C=C\COC(=O)CCCCCN(CCCC(=O)OC(CCCCCCC)CCCCCCC)C(=O)SCCN(C)C]

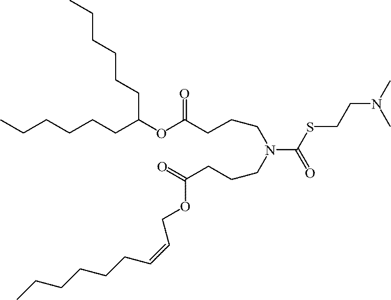 [SMILES: CCCCCC/C=C\COC(=O)CCCN(CCCC(=O)OC(CCCCCC)CCCCCC)C(=O)SCCN(C)C]

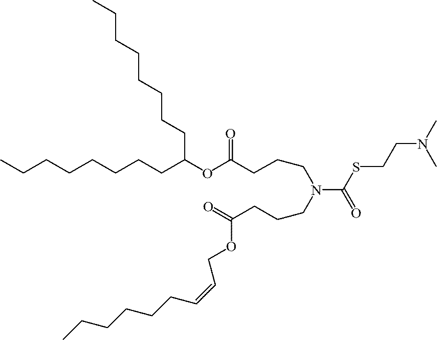 [SMILES: CCCCCC/C=C\COC(=O)CCCN(CCCC(=O)OC(CCCCCCCC)CCCCCCCC)C(=O)SCCN(C)C]

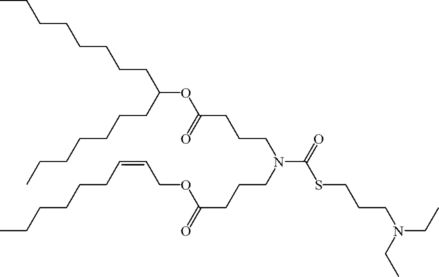 [SMILES: CCCCCC/C=C\COC(=O)CCCN(CCCC(=O)OC(CCCCCCC)CCCCCCCC)C(=O)SCCCN(CC)CC]

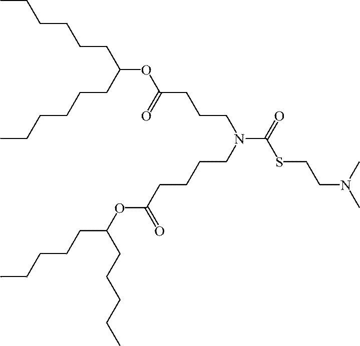 [SMILES: CCCCCCC(CCCCCC)OC(=O)CCCN(CCCCC(=O)OC(CCCCC)CCCCC)C(=O)SCCN(C)C]

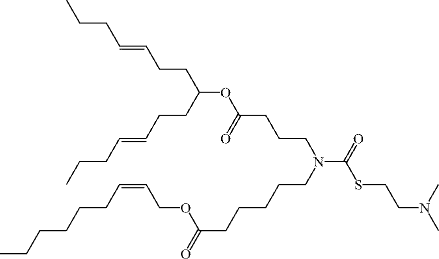 [SMILES: CCC/C=C/CCC(CC/C=C/CCC)OC(=O)CCCN(CCCCCC(=O)OC/C=C\CCCCCC)C(=O)SCCN(C)C]

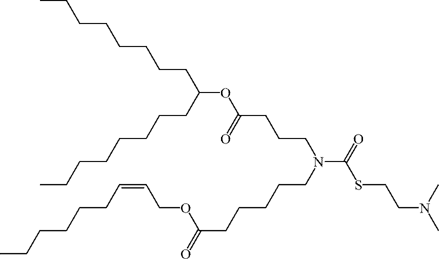 [SMILES: CCCCCC/C=C\COC(=O)CCCCCN(CCCC(=O)OC(CCCCCCCC)CCCCCCCC)C(=O)SCCN(C)C]

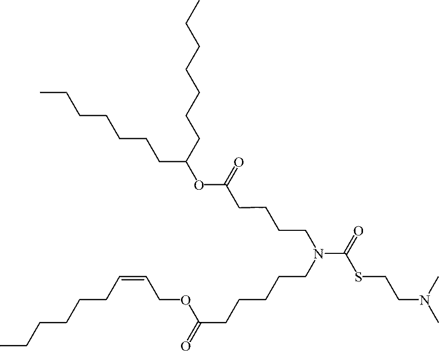 [SMILES: CCCCCC/C=C\COC(=O)CCCCCN(CCCCC(=O)OC(CCCCCCC)CCCCCCC)C(=O)SCCN(C)C]

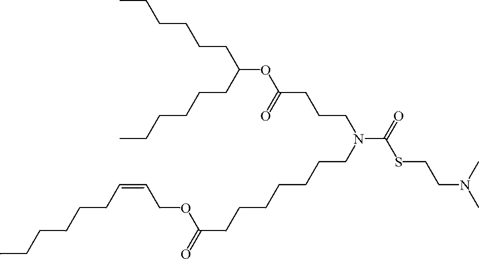 [SMILES: CCCCCC/C=C\COC(=O)CCCCCCCN(CCCC(=O)OC(CCCCCC)CCCCCC)C(=O)SCCN(C)C]

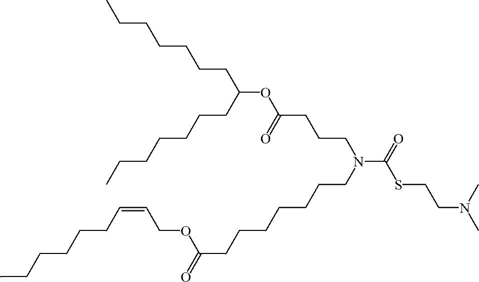 [SMILES: CCCCCC/C=C\COC(=O)CCCCCCCN(CCCC(=O)OC(CCCCCCC)CCCCCCC)C(=O)SCCN(C)C]

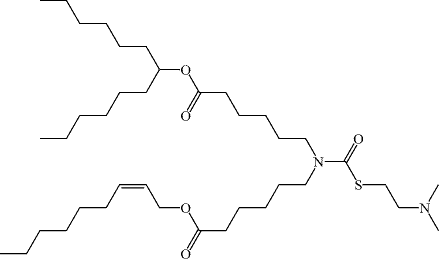 [SMILES: CCCCCC/C=C\COC(=O)CCCCCN(CCCCCC(=O)OC(CCCCCC)CCCCCC)C(=O)SCCN(C)C]

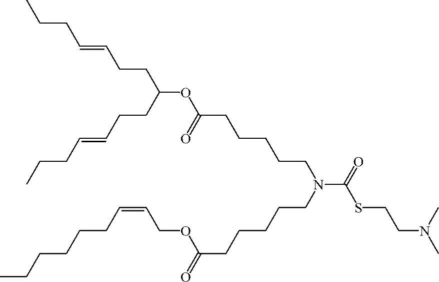 [SMILES: CCC/C=C/CCC(CC/C=C/CCC)OC(=O)CCCCCN(CCCCCC(=O)OC/C=C\CCCCCC)C(=O)SCCN(C)C]

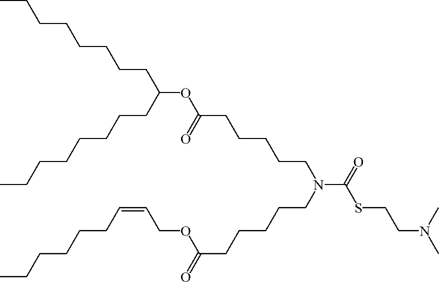 [SMILES: CCCCCC/C=C\COC(=O)CCCCCN(CCCCCC(=O)OC(CCCCCCCC)CCCCCCCC)C(=O)SCCN(C)C]

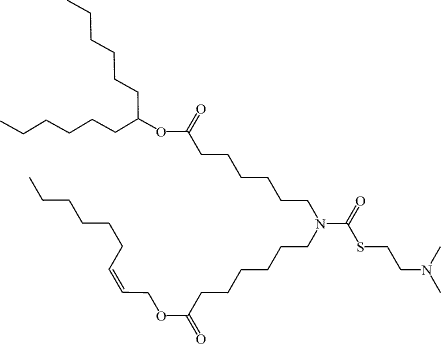 [SMILES: CCCCCC/C=C\COC(=O)CCCCCCN(CCCCCCC(=O)OC(CCCCCC)CCCCCC)C(=O)SCCN(C)C]

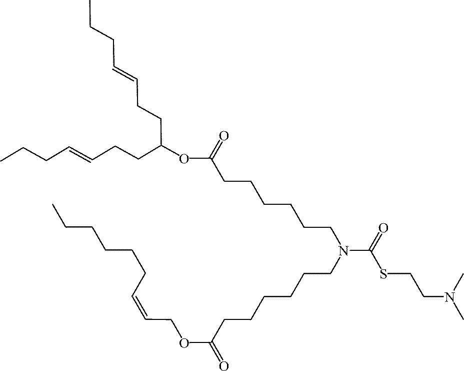 [SMILES: CCC/C=C/CCC(CC/C=C/CCC)OC(=O)CCCCCCN(CCCCCCC(=O)OC/C=C\CCCCCC)C(=O)SCCN(C)C]

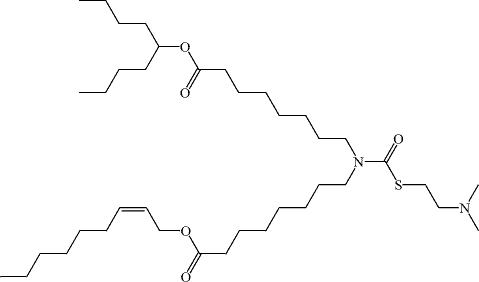 [SMILES: CCCCCC/C=C\COC(=O)CCCCCCCN(CCCCCCCC(=O)OC(CCCC)CCCC)C(=O)SCCN(C)C]

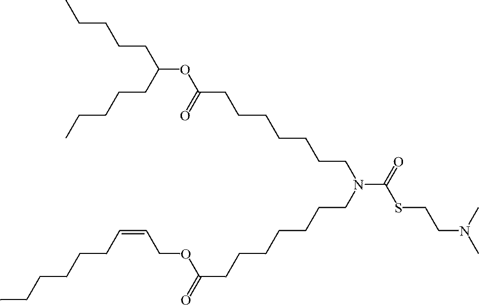 [SMILES: CCCCCC/C=C\COC(=O)CCCCCCCN(CCCCCCCC(=O)OC(CCCCC)CCCCC)C(=O)SCCN(C)C]

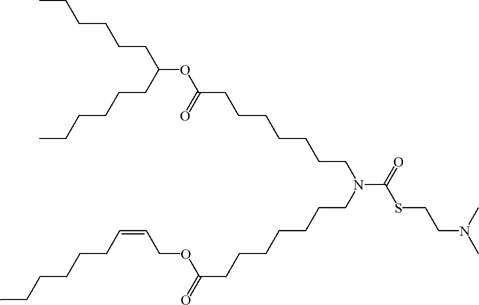 [SMILES: CCCCCC/C=C\COC(=O)CCCCCCCN(CCCCCCCC(=O)OC(CCCCCC)CCCCCC)C(=O)SCCN(C)C]

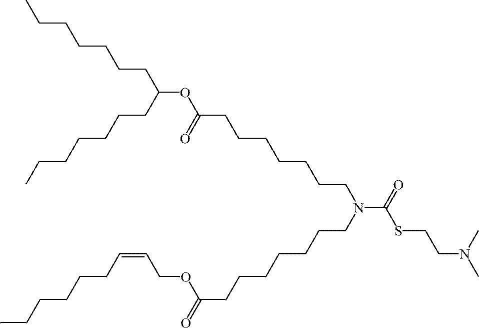 [SMILES: CCCCCC/C=C\COC(=O)CCCCCCCN(CCCCCCCC(=O)OC(CCCCCCC)CCCCCCC)C(=O)SCCN(C)C]

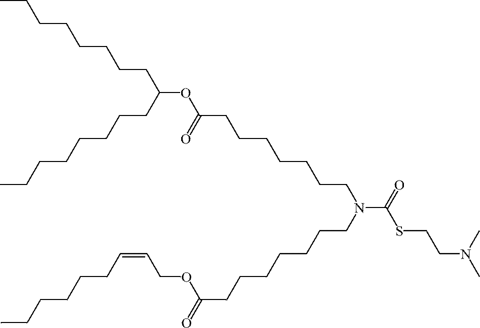 [SMILES: CCCCCC/C=C\COC(=O)CCCCCCCN(CCCCCCCC(=O)OC(CCCCCCCC)CCCCCCCC)C(=O)SCCN(C)C]

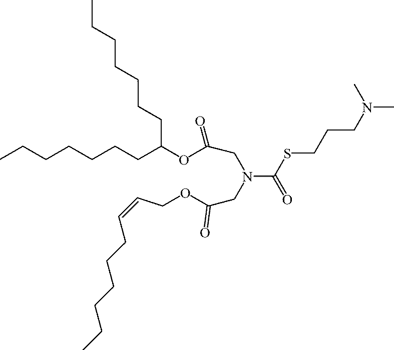 [SMILES: CCCCCC/C=C\COC(=O)CN(CC(=O)OC(CCCCCCC)CCCCCCC)C(=O)SCCCN(C)C]

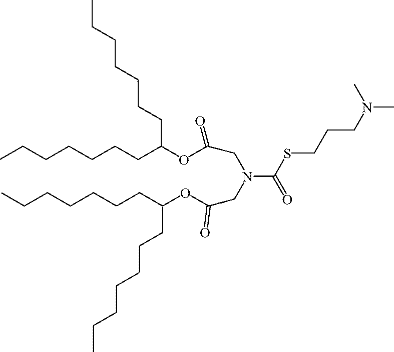 [SMILES: CCCCCCCC(CCCCCCC)OC(=O)CN(CC(=O)OC(CCCCCCC)CCCCCCC)C(=O)SCCCN(C)C]

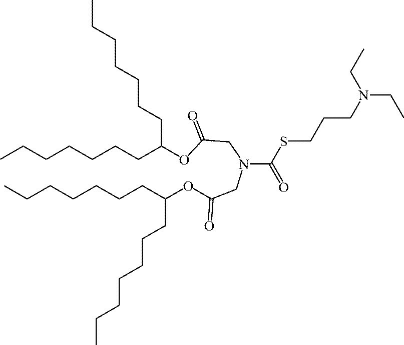 [SMILES: CCCCCCCC(CCCCCCC)OC(=O)CN(CC(=O)OC(CCCCCCC)CCCCCCC)C(=O)SCCCN(CC)CC]

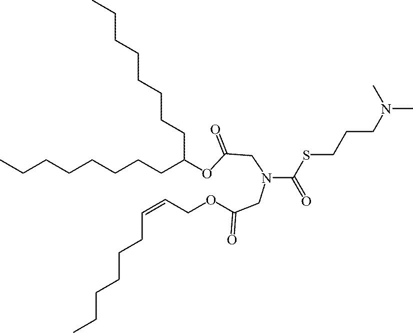 [SMILES: CCCCCC/C=C\COC(=O)CN(CC(=O)OC(CCCCCCCC)CCCCCCCC)C(=O)SCCCN(C)C]

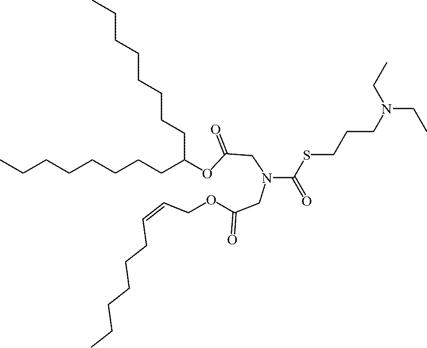 [SMILES: CCCCCC/C=C\COC(=O)CN(CC(=O)OC(CCCCCCCC)CCCCCCCC)C(=O)SCCCN(CC)CC]

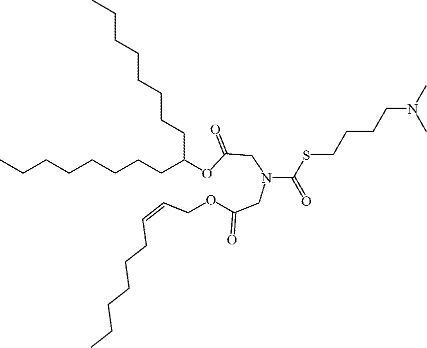 [SMILES: CCCCCC/C=C\COC(=O)CN(CC(=O)OC(CCCCCCCC)CCCCCCCC)C(=O)SCCCCN(C)C]

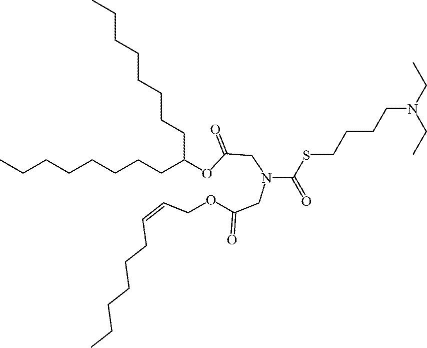 [SMILES: CCCCCC/C=C\COC(=O)CN(CC(=O)OC(CCCCCCCC)CCCCCCCC)C(=O)SCCCCN(CC)CC]

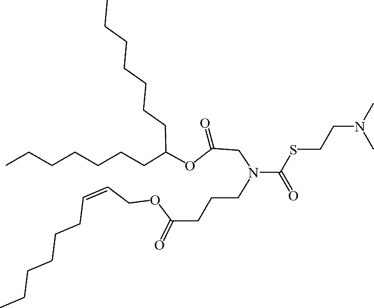 [SMILES: CCCCCC/C=C\COC(=O)CCCN(CC(=O)OC(CCCCCCC)CCCCCCC)C(=O)SCCN(C)C]

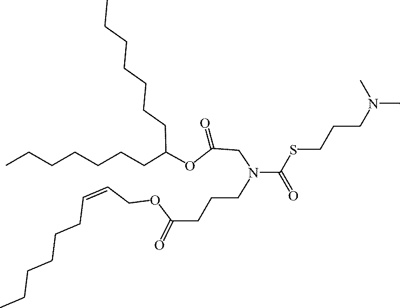 [SMILES: CCCCCC/C=C\COC(=O)CCCN(CC(=O)OC(CCCCCCC)CCCCCCC)C(=O)SCCCN(C)C]

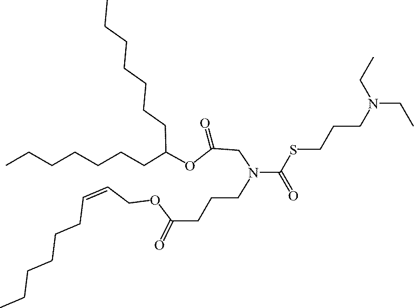 [SMILES: CCCCCC/C=C\COC(=O)CCCN(CC(=O)OC(CCCCCCC)CCCCCCC)C(=O)SCCCN(CC)CC]

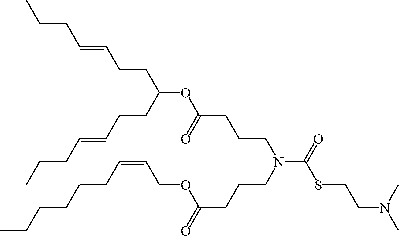 [SMILES: CCC/C=C/CCC(CC/C=C/CCC)OC(=O)CCCN(CCCC(=O)OC/C=C\CCCCCC)C(=O)SCCN(C)C]

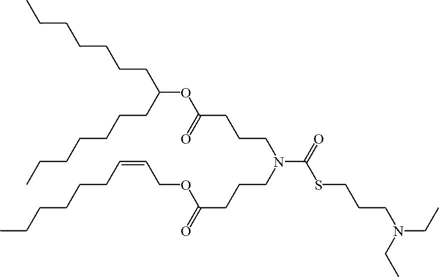 [SMILES: CCCCCC/C=C\COC(=O)CCCN(CCCC(=O)OC(CCCCCCC)CCCCCCC)C(=O)SCCCN(CC)CC]

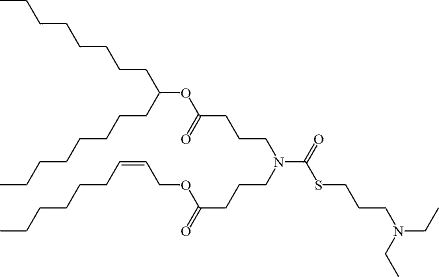 [SMILES: CCCCCC/C=C\COC(=O)CCCN(CCCC(=O)OC(CCCCCCCC)CCCCCCCC)C(=O)SCCCN(CC)CC]

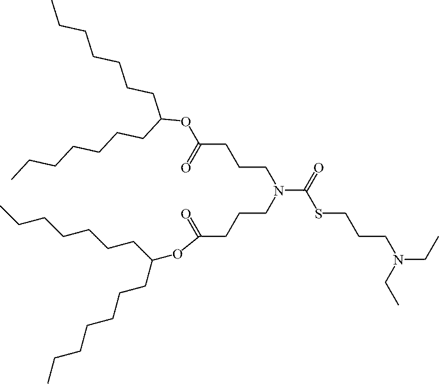 [SMILES: CCCCCCCC(CCCCCCC)OC(=O)CCCN(CCCC(=O)OC(CCCCCCC)CCCCCCC)C(=O)SCCCN(CC)CC]

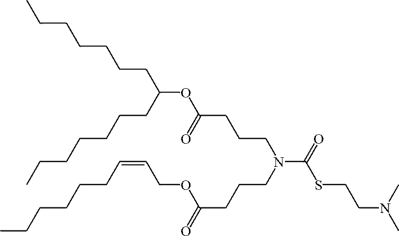 [SMILES: CCCCCC/C=C\COC(=O)CCCN(CCCC(=O)OC(CCCCCCC)CCCCCCC)C(=O)SCCN(C)C]

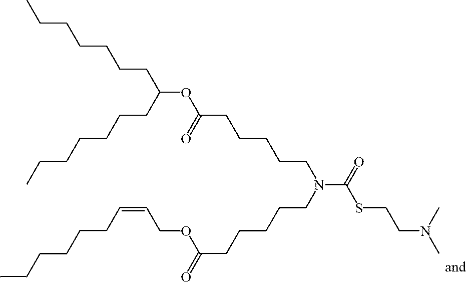

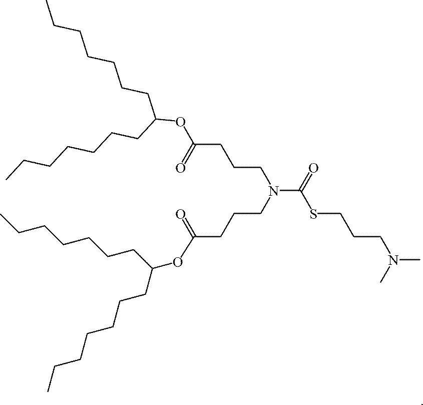

In some embodiments, the lipid formulation comprises an ionizable cationic lipid having a structure selected from

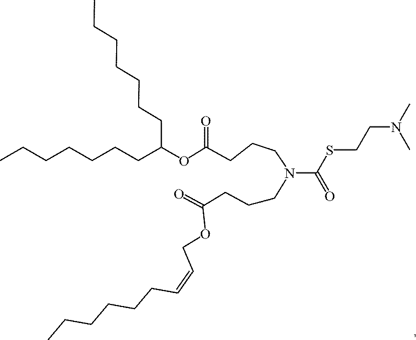

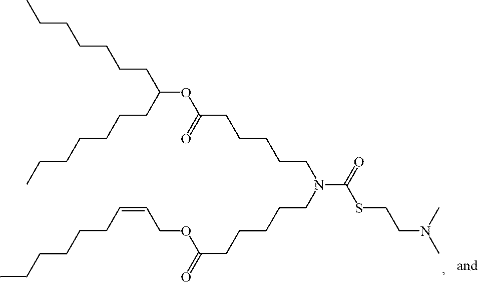

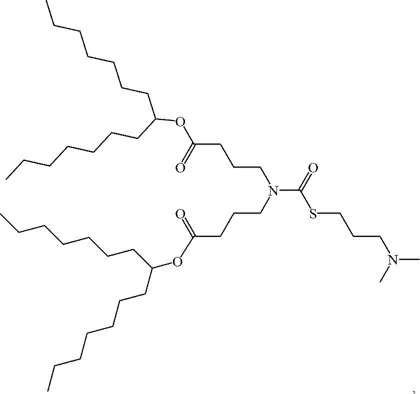

or a pharmaceutically acceptable salt thereof.

In embodiments, any one or more lipids recited herein may be expressly excluded.

### Helper Lipids and Sterols

The UNA oligomer-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (see, for example, Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 Jan; 9(1): 105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (see, for example, J. R. Soc. Interface. 2012 Mar 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′- hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the neutral lipid present in the lipid formulation comprises or consists of one or more phospholipids, e.g., a cholesterol-free lipid formulation. In yet other embodiments, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 2 mol% to about 20 mol%, from about 3 mol% to about 18 mol%, from about 4 mol% to about 16 mol%, about 5 mol% to about 14 mol%, from about 6 mol% to about 12 mol%, from about 5 mol% to about 10 mol%, from about 5 mol% to about 9 mol%, or about 2 mol%, about 3 mol%, about 4 mol%, about 5 mol%, about 6 mol%, about 7 mol%, about 8 mol%, about 9 mol%, about 10 mol%, about 11 mol%, or about 12 mol% (or any fraction thereof or the range therein) of the total lipid present in the lipid formulation.

The cholesterol or cholesterol derivative in the lipid formulation may comprise up to about 40 mol%, about 45 mol%, about 50 mol%, about 55 mol%, or about 60 mol% of the total lipid present in the lipid formulation. In some embodiments, the cholesterol or cholesterol derivative comprises about 15 mol% to about 45 mol%, about 20 mol% to about 40 mol%, about 25 mol% to about 35 mol%, or about 28 mol% to about 35 mol%; or about 25 mol%, about 26 mol%, about 27 mol%, about 28 mol%, about 29 mol%, about 30 mol%, about 31 mol%, about 32 mol%, about 33 mol%, about 34 mol%, about 35 mol%, about 36 mol%, or about 37 mol% of the total lipid present in the lipid formulation.

The percentage of helper lipid present in the lipid formulation is a target amount, and the actual amount of helper lipid present in the formulation may vary, for example, by ± 5 mol%.

A lipid formulation containing a cationic lipid compound or ionizable cationic lipid compound may be on a molar basis about 30-70% cationic lipid compound, about 25-40 % cholesterol, about 2-15% helper lipid, and about 0.5-5% of a polyethylene glycol (PEG) lipid, wherein the percent is of the total lipid present in the formulation. In some embodiments, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1- 15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (see, for example, Front Pharmacol. 2015 Dec 1; 6:286).

In a preferred embodiment, the lipid conjugate is a PEG-lipid. The inclusion of polyethylene glycol (PEG) in a lipid formulation as a coating or surface ligand, a technique referred to as PEGylation, helps to protects nanoparticles from the immune system and their escape from RES uptake (see, for example, Nanomedicine (Lond). 2011 Jun; 6(4):715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol%) and will shift to brush conformation as the content of PEG-DSPE is increased past a certain level (see, for example, Journal of Nanomaterials. 2011;2011:12). It has been shown that increased PEGylation leads to a significant increase in the circulation half-life of lipid formulations (see, for example, Annu. Rev. Biomed. Eng. 2011 Aug 15; 13():507-30; J. Control Release. 2010 Aug 3; 145(3):178-81).

Suitable examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol- succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH₂).

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In preferred embodiments, the PEG moiety has an average molecular weight of about 2,000 daltons or about 750 daltons. The average molecular weight may be any value or subvalue within the recited ranges, including endpoints.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In a preferred embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, as well as combinations thereof (such as a linker containing both a carbamate linker moiety and an amido linker moiety). In a preferred embodiment, a carbamate linker is used to couple the PEG to the lipid.

In other embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

In some embodiments, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 or about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some embodiments, the lipid conjugate can comprise a mixture of a compound of Formula (II), (III), and or (IV) as described herein in combination with a PEG-lipid. In some embodiments, the lipid conjugate can comprise a lipid having one or more GalNAc moieties conjugated thereto.

In some embodiments, the lipid conjugate (e.g., PEG-lipid) comprises from about 0.1 mol% to about 2 mol%, from about 0.5 mol% to about 2 mol%, from about 1 mol% to about 2 mol%, from about 0.6 mol% to about 1.9 mol%, from about 0.7 mol% to about 1.8 mol%, from about 0.8 mol% to about 1.7 mol%, from about 0.9 mol% to about 1.6 mol%, from about 0.9 mol% to about 1.8 mol%, from about 1 mol% to about 1.8 mol%, from about 1 mol% to about 1.7 mol%, from about 1.2 mol% to about 1.8 mol%, from about 1.2 mol% to about 1.7 mol%, from about 1.3 mol% to about 1.6 mol%, or from about 1.4 mol% to about 1.6 mol% (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ± 0.5 mol%. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells’ endocytic mechanisms where the contents of the lipid delivery vehicle are delivered to the cytosol of the target cell (see, for example, Nucleic Acid Therapeutics, 28(3):146-157, 2018). Specifically, in the case of a trinucleotide expansion interfering UNA oligomer-lipid formulation described herein, the UNA oligomer-lipid formulation enters cells through receptor mediated endocytosis. Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For lipid formulations comprising a GalNAc moiety, Tris-GalNAc binds to the Asialoglycoprotein receptor that is highly expressed on hepatocytes resulting in rapid endocytosis. While the exact mechanism of escape across the endosomal lipid bilayer membrane remains unknown, sufficient amounts of siRNAs enter the cytoplasm to induce robust, target selective RNAi responses in vivo.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid (see, for example, Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly, lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

### Pharmaceutical Compositions and Methods of Treatment

To facilitate RNA interference in vivo, the nucleic acid lipid formulation delivery vehicles described herein can be combined with one or more additional nucleic acids, carriers, targeting ligands or stabilizing reagents, or in pharmacological compositions where it is mixed with suitable excipients. Techniques for formulation and administration of drugs may be found in “Remington’s Pharmaceutical Sciences,” Mack Publishing Co., Easton, Pa., latest edition. Preferably, the nucleic acid lipid formulation is a UNA oligomer-lipid nanoparticle formulation as described herein. In some embodiments, the pharmaceutical composition further comprises pharmaceutically acceptable excipients. Pharmaceutical compositions disclosed herein preferably facilitate RNA interference in vivo.

The lipid formulations and pharmaceutical compositions of the present disclosure may be administered and dosed in accordance with current medical practice, taking into account the clinical condition of the subject, the site and method of administration, the scheduling of administration, the subject’s age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient knockdown of the activity of a target.

In some embodiments, the pharmaceutical compositions described are administered systemically. Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously. In some embodiments, the administration results in delivery of the UNA oligomer to a brain cell.

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or UNA oligomer); (4) alter the biodistribution (e.g., target the polynucleotide, primary construct, or UNA oligomer to specific tissues or cell types); (5) increase the knockdown activity of the UNA oligomer in vivo; and/or (6) alter the selectivity of the UNA oligomer of a target gene in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or mRNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the UNA oligomer of the present disclosure may be formulated using self-assembled nucleic acid nanoparticles.

Various excipients for formulating pharmaceutical compositions and techniques for preparing the composition are known in the art (see, for example, Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

A dosage form of the composition of this disclosure can be solid, which can be reconstituted in a liquid prior to administration. The solid can be administered as a powder. The solid can be in the form of a capsule, tablet, or gel. In some embodiments, the pharmaceutical composition comprises a nucleic acid lipid formulation that has been lyophilized.

In a preferred embodiment, the dosage form of the pharmaceutical compositions described herein can be a liquid suspension of UNA oligomer-lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about 70° C. In some embodiments, the suspension is diluted with sterile water during intravenous administration. In some embodiments, intravenous administration comprises diluting the suspension with about 2 volumes to about 6 volumes of sterile water. In some embodiments, the suspension comprises about 0.1 mg to about 3.0 mg UNA oligomer/mL, about 15 mg/mL to about 25 mg/mL of an ionizable cationic lipid, about 0.5 mg/mL to about 2.5 mg/mL of a PEG-lipid, about 1.8 mg/mL to about 3.5 mg/mL of a helper lipid, about 4.5 mg/mL to about 7.5 mg/mL of a cholesterol, about 7 mg/mL to about 15 mg/mL of a buffer, about 2.0 mg/mL to about 4.0 mg/mL of NaCl, about 70 mg/mL to about 110 mg/mL of sucrose, and about 50 mg/mL to about 70 mg/mL of glycerol. In some embodiments, a lyophilized UNA oligomer lipid nanoparticle formulation can be resuspended in a buffer as described herein.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the UNA oligomer -lipid formulation may be administered in a time release formulation, for example in a composition which includes a slow release polymer. The active agent can be prepared with carriers that will protect against rapid release, for example a controlled release vehicle such as a polymer, microencapsulated delivery system, or a bioadhesive gel. Prolonged delivery of the UNA oligomer, in various compositions of the disclosure can be brought about by including in the composition agents that delay absorption, for example, aluminum monostearate hydrogels and gelatin.

In some embodiments, the compositions of the disclosure are administered to a subject such that a UNA oligomer concentration of at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight is administered in a single dose or as part of single treatment cycle. In some embodiments, the compositions of the disclosure are administered to a subject such that a total amount of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, or at least about 125 mg UNA oligomer is administered in one or more doses up to a maximum dose of about 300 mg, about 350 mg, about 400 mg, about 450 mg, or about 500 mg UNA oligomer.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

Alternatively, the compositions of the present disclosure may be administered in a local rather than systemic manner, for example, via injection of the pharmaceutical composition directly into a targeted tissue, preferably in a depot or sustained release formulation. Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present disclosure complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

In some embodiments, this disclosure provides a method of treating a disease or disorder in a mammalian subject. A therapeutically effective amount of a composition of this disclosure containing a nucleic may be administered to a subject having a disease or disorder associated with expression or overexpression of a gene that can be reduced, decreased, downregulated, or silenced by the composition.

The oligomer-lipid compositions may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the biologically active agent.

### Combinations

The UNA oligomer and formulations thereof described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. In general, it is expected that agents utilized in combination with the presently disclosed UNA oligomer and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### Definitions

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The phrases “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

The terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

The terms “antigens of interest” or “desired antigens” include those proteins and other biomolecules provided herein that are immunospecifically bound by the antibodies and fragments, mutants, variants, and alterations thereof described herein. Examples of antigens of interest include, but are not limited to, insulin, insulin-like growth factor, hGH, tPA, cytokines, such as interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TRAIL; G-CSF, GM-CSF, M-CSF, MCP-1 and VEGF.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the exemplary alkyl substituent groups described herein.

The term “alkyl,” as used herein, is inclusive of both straight chain and branched chain saturated groups from 1 to 20 carbons (e.g., from 1 to 10 or from 1 to 6), unless otherwise specified. Alkyl groups are exemplified by methyl, ethyl, n- and iso-propyl, n-, sec-,iso- and tert-butyl, neopentyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) COO-aryl-C₁₋₆ alkoxy; (5) azido; (6) halo; (7) (C₂₋₉ heterocyclyl)oxy; (8) hydroxy, optionally substituted with an O-protecting group; (9) nitro; (10) oxo (e.g., carboxyaldehyde or acyl); (11) C₁₋₇ spirocyclyl; (12) thioalkoxy; (13) thiol; (14) —CO₂R^{A′}, optionally substituted with an O-protecting group and where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ to aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ to aryl, and (d) C₁₋₆ alkyl-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alkyl-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alkyl-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of — NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀, alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of -NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein. For example, the alkyl group of a C₁-alkaryl can be further substituted with an oxo group to afford the respective aryloyl substituent.

The term “lower alkyl” means a group having one to six carbons in the chain which chain may be straight or branched. Non-limiting examples of suitable alkyl groups include methyl, ethyl, n-propyl, isopropyl, n-butyl, t-butyl, n-pentyl, and hexyl.

The term “alkylsulfinyl,” as used herein, represents an alkyl group attached to the parent molecular group through an —S(O)— group. Exemplary unsubstituted alkylsulfinyl groups are from 1 to 6, from 1 to 10, or from 1 to 20 carbons. In some embodiments, the alkyl group can be further substituted with 1, 2, 3, or 4 substituent groups as defined herein.

The term “alkylsulfinylalkyl,” as used herein, represents an alkyl group, as defined herein, substituted by an alkylsulfinyl group. Exemplary unsubstituted alkylsulfinylalkyl groups are from 2 to 12, from 2 to 20, or from 2 to 40 carbons. In some embodiments, each alkyl group can be further substituted with 1, 2, 3, or 4 substituent groups as defined herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the exemplary alkyl substituent groups described herein.

The term “alkynyloxy” represents a chemical substituent of formula —OR, where R is a C₂₋₂₀ alkynyl group (e.g., C₂₋₆ or C₂₋₁₀ alkynyl), unless otherwise specified. Exemplary alkynyloxy groups include ethynyloxy, propynyloxy, and the like. In some embodiments, the alkynyl group can be further substituted with 1, 2, 3, or 4 substituent groups as defined herein (e.g., a hydroxy group).

The term “amidine,” as used herein, represents a —C(═NH)NH₂ group.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkylcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkylheterocyclyl (e.g., alkylheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the disclosure can be an unsubstituted amino (i.e., —NH₂) or a substituted amino (i.e., —N(R′)₂). In a preferred embodiment, amino is —NH₂ or —NHR^{N1}, wherein R^{N1} is, independently, OH, NO₂, NH₂, NR^{N2} ₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₁₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). In some embodiments, the amino acid is attached to the parent molecular group by a carbonyl group, where the side chain or amino group is attached to the carbonyl group. Exemplary side chains include an optionally substituted alkyl, aryl, heterocyclyl, alkylaryl, alkylheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of — NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alkyl-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alkyl-C₆₋₁₀ aryl, and (d) hydroxy; (17) — SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alkyl-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂-₂₀ alkenyl (e.g., C₂-₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein.

The term “aminoalkoxy,” as used herein, represents an alkoxy group, as defined herein, substituted by an amino group, as defined herein. The alkyl and amino each can be further substituted with 1, 2, 3, or 4 substituent groups as described herein for the respective group (e.g., CO₂R^{A}, where R^{A′} is selected from the group consisting of (a) C₁-₆ alkyl, (b) C₆₋₁₀ aryl, (c) hydrogen, and (d) C₁₋₆ alkyl-C₆₋₁₀ aryl, e.g., carboxy).

The term “aminoalkyl,” as used herein, represents an alkyl group, as defined herein, substituted by an amino group, as defined herein. The alkyl and amino each can be further substituted with 1, 2, 3, or 4 substituent groups as described herein for the respective group (e.g., CO₂R^{A}, where R^{A′} is selected from the group consisting of (a) C₁-₆ alkyl, (b) C₆₋₁₀ aryl, (c) hydrogen, and (d) C₁₋₆ alkyl-C₆₋₁₀ aryl, e.g., carboxy, and/or an N-protecting group).

The term “aminoalkenyl,” as used herein, represents an alkenyl group, as defined herein, substituted by an amino group, as defined herein. The alkenyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “aminoalkynyl,” as used herein, represents an alkynyl group, as defined herein, substituted by an amino group, as defined herein. The alkynyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, anthracenyl, phenanthrenyl, fluorenyl, indanyl, indenyl, and the like, and may be optionally substituted with 1, 2, 3, 4, or 5 substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alkyl-C₆₋₁₀ aryl; (8) azido; (9) C₃₋₈ cycloalkyl; (10) C₁₋₆ alkyl-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alkyl-C₁₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁ aryl, and (d) C₁₋₆ alkyl-C₆₋₁₀ aryl; (19) — (CH₂)_{q}SO₂R^{D′}, where q is an integer from zero to four and where R^{D′} is selected from the group consisting of (a) alkyl, (b) C₆₋₁₀ aryl, and (c) alkyl-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alkyl-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alkyl-C₁₋₁₂heterocyclyl (e.g., C₁₋₆ alkyl-C₁₋₁₂ heteroaryl); (26) C₂₋₂₀ alkenyl; and (27) C₁₋₂₀ alkynyl. In some embodiments, each of these groups can be further substituted as described herein. For example, the alkyl group of a C₁-alkylaryl or a C₁-alkylheterocyclyl can be further substituted with an oxo group to afford the respective aryloyl and (heterocyclyl)oyl substituent group.

The term “arylalkoxy,” as used herein, represents an alkylaryl group, as defined herein, attached to the parent molecular group through an oxygen atom. Exemplary unsubstituted arylalkoxy groups include from 7 to 30 carbons (e.g., from 7 to 16 or from 7 to 20 carbons, such as C₆₋₁₀ aryl-C₁₋₆ alkoxy, C₆₋₁₀ aryl-C₁₋₁₀ alkoxy, or C₆₋₁₀ aryl-C₁₋₂₀ alkoxy). In some embodiments, the arylalkoxy group can be substituted with 1, 2, 3, or 4 substituents as defined herein

The term “arylalkoxycarbonyl,” as used herein, represents an arylalkoxy group, as defined herein, attached to the parent molecular group through a carbonyl (e.g., — C(O)—O—alkyl-aryl). Exemplary unsubstituted arylalkoxy groups include from 8 to 31 carbons (e.g., from 8 to 17 or from 8 to 21 carbons, such as C₆₋₁₀ aryl-C₁₋₆ alkoxy-carbonyl, C₆₋₁₀ aryl-C₁₋₁₀ alkoxy-carbonyl, or C₆₋₁₀ aryl-C₁₋₂₀ alkoxy-carbonyl). In some embodiments, the arylalkoxycarbonyl group can be substituted with 1, 2, 3, or 4 substituents as defined herein.

The term “aryloxy” represents a chemical substituent of formula —OR′, where R′ is an aryl group of 6 to 18 carbons, unless otherwise specified. In some embodiments, the aryl group can be substituted with 1, 2, 3, or 4 substituents as defined herein.

The term “aryloyl,” as used herein, represents an aryl group, as defined herein, that is attached to the parent molecular group through a carbonyl group. Exemplary unsubstituted aryloyl groups are of 7 to 11 carbons. In some embodiments, the aryl group can be substituted with 1, 2, 3, or 4 substituents as defined herein.

The phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The terms “include,” “have,” or the like is used in the description or the claims, such term is intended to be inclusive in a manner similar to the term “comprise” as “comprise” is interpreted when employed as a transitional word in a claim.

The terms “about,” “substantially,” and “approximately” may provide an industry-accepted tolerance for their corresponding terms and/or relativity between items, such as from less than one percent to five percent.

The term “azido” represents an —N₃ group, which can also be represented as —N═N═N.

The term “bicyclic,” as used herein, refer to a structure having two rings, which may be aromatic or non-aromatic. Bicyclic structures include spirocyclyl groups, as defined herein, and two rings that share one or more bridges, where such bridges can include one atom or a chain including two, three, or more atoms. Exemplary bicyclic groups include a bicyclic carbocyclyl group, where the first and second rings are carbocyclyl groups, as defined herein; a bicyclic aryl groups, where the first and second rings are aryl groups, as defined herein; bicyclic heterocyclyl groups, where the first ring is a heterocyclyl group and the second ring is a carbocyclyl (e.g., aryl) or heterocycyl (e.g., heteroaryl) group; and bicyclic heteroaryl groups, where the first ring is a heteroaryl group and the second ring is a carbocyclyl (e.g., aryl) or heterocyclyl (e.g., heteroaryl) group. In some embodiments, the bicyclic group can be substituted with 1, 2, 3, or 4 substituents as defined herein for cycloalkyl, heterocyclyl, and aryl groups.

The term “boranyl,” as used herein, represents —B(R^{B1})₃, where each R^{B1} is, independently, selected from the group consisting of H and optionally substituted alkyl. In some embodiments, the boranyl group can be substituted with 1, 2, 3, or 4 substituents as defined herein for alkyl.

The term “biocompatible” means compatible with living cells, tissues, organs or systems posing little to no risk of injury, toxicity or rejection by the immune system.

The term “biodegradable” means capable of being broken down into innocuous products by the action of living things.

The phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present disclosure may be considered biologically active if even a portion of the polynucleotide is biologically active or mimics an activity considered biologically relevant.

The terms “carbocyclic” and “carbocyclyl,” as used herein, refer to an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbamoyl,” as used herein, represents —C(O)—N(R^{N1})₂, where the meaning of each R^{N1} is found in the definition of “amino” provided herein.

The term “carbamoylalkyl,” as used herein, represents an alkyl group, as defined herein, substituted by a carbamoyl group, as defined herein. The alkyl group can be further substituted with 1, 2, 3, or 4 substituent groups as described herein.

The term “carbamyl,” as used herein, refers to a carbamate group having the structure —NR^{N1}C(═O)OR or —OC(═O)N(R^{N1})₂, where the meaning of each R^{N1} is found in the definition of “amino” provided herein, and R is alkyl, cycloalkyl, alkylcycloalkyl, aryl, alkylaryl, heterocyclyl (e.g., heteroaryl), or alkylheterocyclyl (e.g., alkylheteroaryl), as defined herein.

The term “carbonyl,” as used herein, represents a C(O) group, which can also be represented as C═O.

The term “carboxyaldehyde” represents an acyl group having the structure —C(O)H.

The term “carboxy,” as used herein, means —CO₂H.

The term “carboxyalkoxy,” as used herein, represents an alkoxy group, as defined herein, substituted by a carboxy group, as defined herein. The alkoxy group can be further substituted with 1, 2, 3, or 4 substituent groups as described herein for the alkyl group, and the carboxy group can be optionally substituted with one or more O-protecting groups.

The term “carboxyalkyl,” as used herein, represents an alkyl group, as defined herein, substituted by a carboxy group, as defined herein. The alkyl group can be further substituted with 1, 2, 3, or 4 substituent groups as described herein, and the carboxy group can be optionally substituted with one or more O-protecting groups.

The term “carboxyaminoalkyl,” as used herein, represents an aminoalkyl group, as defined herein, substituted by a carboxy, as defined herein. The carboxy, alkyl, and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group, and/or an O-protecting group).

The term “comprising” is intended to be open and permits but does not require the inclusion of additional elements or steps. When the term “comprising” is used herein, the term “consisting of” is thus also encompassed and disclosed.

The term “composition” means a product comprising the specified ingredients in the specified amounts, as well as any product that results, directly or indirectly, from combination of the specified ingredients in the specified amounts.

The term “in combination with” means the administration of a pharmaceutical composition or UNA oligomer of the present disclosure with other medicaments in the methods of treatment of this disclosure, means-that the pharmaceutical composition or UNA oligomer of the present disclosure and the other medicaments are administered sequentially or concurrently in separate dosage forms, or are administered concurrently in the same dosage form.

The term “cyano,” as used herein, represents an —CN group.

The term “cycloalkoxy” represents a chemical substituent of formula —OR, where R is a C₃₋₈ cycloalkyl group, as defined herein, unless otherwise specified. The cycloalkyl group can be further substituted with 1, 2, 3, or 4 substituent groups as described herein. Exemplary unsubstituted cycloalkoxy groups are from 3 to 8 carbons. In some embodiment, the cycloalkyl group can be further substituted with 1, 2, 3, or 4 substituent groups as described herein.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this disclosure can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₂ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alkyl-C₆₋₁₀ aryl; (8) azido; (9) C₃₋₈ cycloalkyl; (10) C₁₋₆ alkyl-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alkyl-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alkyl-C₆₋₁₀ aryl; (19) — (CH₂)_{q}SO₂R^{D′}, where q is an integer from zero to four and where R^{D′} is selected from the group consisting of (a) C₆₋₁₀ alkyl, (b) C₆₋₁₀ aryl, and (c) C₁₋₆ alkyl-C₆₋₁₀ aryl; (20) — (CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alkyl-C₁₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alkyl-C₁₋₁₂ heterocyclyl (e.g., C₁₋₆ alkyl-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkyl group of a C₁-alkaryl or a C₁-alkylheterocyclyl can be further substituted with an oxo group to afford the respective aryloyl and (heterocyclyl)oyl substituent group.

The term “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

The term “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

The term “diastereomer,” as used herein means stereoisomers that are not mirror images of one another and are non-superimposable on one another.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R¹ and R² are both stearoyl (i.e., distearoyl).

The term “dialkyloxypropyl” or “DAA” includes a compound having 2 alkyl chains, R and R, both of which have independently between 2 and 30 carbons. The alkyl groups can be saturated or have varying degrees of unsaturation.

The term “delivery” refers to the act or manner of delivering a compound, substance, entity, moiety, cargo or payload.

The term “delivery agent” refers to any substance that facilitates, at least in part, the in vivo delivery of a polynucleotide to targeted cells.

The terms “destable,” “destabilize,” or “destabilizing region” means a region or molecule that is less stable than a starting, wild-type or native form of the same region or molecule.

The term “detectable label” refers to one or more markers, signals, or moieties, which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

The term “digest” means to break apart into smaller pieces or components. When referring to polypeptides or proteins, digestion results in the production of peptides.

The term “distal” means situated away from the center or away from a point or region of interest.

The term “effective amount” of an agent, as used herein, is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

The term “enantiomer,” as used herein, means each individual optically active form of a compound of the disclosure, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “engineered” when they are designed to have a feature or property, whether structural or chemical, that varies from a starting point, wild type or native molecule.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

The term “feature” refers to a characteristic, a property, or a distinctive element.

The term “formulation” includes at least a polynucleotide and a delivery agent.

The term “fragment,” as used herein, refers to a portion. For example, fragments of proteins may comprise polypeptides obtained by digesting full-length protein isolated from cultured cells.

The term “functional” biological molecule is a biological molecule in a form in which it exhibits a property and/or activity by which it is characterized.

The term “fully encapsulated” means that the nucleic acid (e.g., siRNA) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” also means that the nucleic acid-lipid particles do not rapidly decompose into their component parts upon in vivo administration.

The terms “halo” and “Halogen”, as used herein, represents a halogen selected from bromine, chlorine, iodine, or fluorine.

The term “haloalkoxy,” as used herein, represents an alkoxy group, as defined herein, substituted by a halogen group (i.e., F, Cl, Br, or I). A haloalkoxy may be substituted with one, two, three, or, in the case of alkyl groups of two carbons or more, four halogens. Haloalkoxy groups include perfluoroalkoxys (e.g., —OCF₃), —OCHF₂, —OCH₂F, —OCCl₃, —OCH₂CH₂Br, —OCH₂CH(CH₂CH₂Br)CH₃, and —OCHICH₃. In some embodiments, the haloalkoxy group can be further substituted with 1, 2, 3, or 4 substituent groups as described herein for alkyl groups.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls (e.g., —CF₃), —CHF₂, —CH₂F, —CCl₃, —CH₂CH₂Br, — CH₂CH(CH₂CH₂Br)CH₃, and —CHICH₃. In some embodiments, the haloalkyl group can be further substituted with 1, 2, 3, or 4 substituent groups as described herein for alkyl groups.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined for a heterocyclyl group.

The term “heterocyclyl,” as used herein represents a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, indazolyl, quinolyl, isoquinolyl, quinoxalinyl, dihydroquinoxalinyl, quinazolinyl, cinnolinyl, phthalazinyl, benzimidazolyl, benzothiazolyl, benzoxazolyl, benzothiadiazolyl, furyl, thienyl, thiazolidinyl, isothiazolyl, triazolyl, tetrazolyl, oxadiazolyl (e.g., 1,2,3-oxadiazolyl), purinyl, thiadiazolyl (e.g., 1,2,3-thiadiazolyl), tetrahydrofuranyl, dihydrofuranyl, tetrahydrothienyl, dihydrothienyl, dihydroindolyl, dihydroquinolyl, tetrahydroquinolyl, tetrahydroisoquinolyl, dihydroisoquinolyl, pyranyl, dihydropyranyl, dithiazolyl, benzofuranyl, isobenzofuranyl, benzothienyl, and the like, including dihydro and tetrahydro forms thereof, where one or more double bonds are reduced and replaced with hydrogens. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H-purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Additional heterocyclics include 3,3a,4,5,6,6a-hexahydro-pyrrolo[3,4-b]pyrrol-(2H)-yl, and 2,5-diazabicyclo[2.2.1]heptan-2-yl, homopiperazinyl (or diazepanyl), tetrahydropyranyl, dithiazolyl, benzofuranyl, benzothienyl, oxepanyl, thiepanyl, azocanyl, oxecanyl, and thiocanyl. Heterocyclic groups also include groups of the Formula

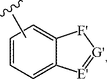

wherein, E′ is selected from the group consisting of—N— and —CH—; F′ is selected from the group consisting of —N═CH—, —NH—CH₂—, —NH—C(O)—, —NH—, —CH═N—, —CH₂—NH—, —C(O)—NH—, —CH═CH—, —CH₂—, —CH₂CH₂—, —CH₂O—, — OCH₂—, —O—, and —S—; and G′ is selected from the group consisting of —CH— and — N—.

Any of the heterocyclyl groups disclosed herein may be optionally substituted with one, two, three, four or five substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alkyl-C₆₋₁₀ aryl; (8) azido; (9) C₃₋₈ cycloalkyl; (10) C₁₋₆ alkyl-C₃₋₈ cycloalkyl; (11) halo; (12) C₁₋₁₂ heterocyclyl (e.g., C₂₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alkyl-C₆₋₁₀ aryl; (18) -(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alkyl-C₆₋₁₀ aryl; (19) —(CH₂)_{q}SO₂R^{D′}, where q is an integer from zero to four and where R^{D′} is selected from the group consisting of (a) C₁₋₆ alkyl, (b) C₆₋₁₀ aryl, and (c) C₁₋₆ alkyl-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alkyl-C₆₋₁₀ aryl; (21) thiol; (22) C₆₋₁₀ aryloxy; (23) C₃₋₈ cycloalkoxy; (24) arylalkoxy; (25) C₁₋₆ alkyl-C₁₋₁₂ heterocyclyl (e.g., C₁₋₆ alkyl-C₁₋₁₂ heteroaryl); (26) oxo; (27) (C₁₋₁₂ heterocyclyl)imino; (28) C₂₋₂₀ alkenyl; and (29) C₂₋₂₀ alkynyl. In some embodiments, each of these groups can be further substituted as described herein. For example, the alkyl group of a C₁-alkylaryl or a C₁-alkylheterocyclyl can be further substituted with an oxo group to afford the respective aryloyl and (heterocyclyl)oyl substituent group.

The term “(heterocyclyl)imino,” as used herein, represents a heterocyclyl group, as defined herein, attached to the parent molecular group through an imino group. In some embodiments, the heterocyclyl group can be substituted with 1, 2, 3, or 4 substituent groups as defined herein.

The term “(heterocyclyl)oxy,” as used herein, represents a heterocyclyl group, as defined herein, attached to the parent molecular group through an oxygen atom. In some embodiments, the heterocyclyl group can be substituted with 1, 2, 3, or 4 substituent groups as defined herein.

The term “(heterocyclyl)oyl,” as used herein, represents a heterocyclyl group, as defined herein, attached to the parent molecular group through a carbonyl group. In some embodiments, the heterocyclyl group can be substituted with 1, 2, 3, or 4 substituent groups as defined herein.

The term “hydrocarbon,” as used herein, represents a group consisting only of carbon and hydrogen atoms.

The term “hydroxy,” as used herein, represents an —OH group. In some embodiments, the hydroxy group can be substituted with 1, 2, 3, or 4 substituent groups (e.g., O-protecting groups) as defined herein for an alkyl.

The term “hydroxyalkenyl,” as used herein, represents an alkenyl group, as defined herein, substituted by one to three hydroxy groups, with the proviso that no more than one hydroxy group may be attached to a single carbon atom of the alkyl group, and is exemplified by dihydroxypropenyl, hydroxyisopentenyl, and the like. In some embodiments, the hydroxyalkenyl group can be substituted with 1, 2, 3, or 4 substituent groups (e.g., O-protecting groups) as defined herein for an alkyl.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like. In some embodiments, the hydroxyalkyl group can be substituted with 1, 2, 3, or 4 substituent groups (e.g., O-protecting groups) as defined herein for an alkyl.

The term “hydroxyalkynyl,” as used herein, represents an alkynyl group, as defined herein, substituted by one to three hydroxy groups, with the proviso that no more than one hydroxy group may be attached to a single carbon atom of the alkyl group. In some embodiments, the hydroxyalkynyl group can be substituted with 1, 2, 3, or 4 substituent groups (e.g., O-protecting groups) as defined herein for an alkyl.

The term “hydrate” means a solvate wherein the solvent molecule is H₂O.

The term “homology” refers to the overall relatedness between polymeric molecules, e.g. between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

The term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

The term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

The term “isomer,” as used herein, means any tautomer, stereoisomer, enantiomer, or diastereomer of any compound of the disclosure. It is recognized that the compounds of the disclosure can have one or more chiral centers and/or double bonds and, therefore, exist as stereoisomers, such as double-bond isomers (i.e., geometric E/Z isomers) or diastereomers (e.g., enantiomers (i.e., (+) or (-)) or cis/trans isomers). According to the disclosure, the chemical structures depicted herein, and therefore the compounds of the disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “nitro,” as used herein, represents an —NO₂ group.

The term “nucleic acid” means deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′—O—methyl ribonucleotides, peptide-nucleic acids (PNAs).

The term “oxo” as used herein, represents ═O.

The term “perfluoroalkyl,” as used herein, represents an alkyl group, as defined herein, where each hydrogen radical bound to the alkyl group has been replaced by a fluoride radical. Perfluoroalkyl groups are exemplified by trifluoromethyl, pentafluoroethyl, and the like.

The term “perfluoroalkoxy,” as used herein, represents an alkoxy group, as defined herein, where each hydrogen radical bound to the alkoxy group has been replaced by a fluoride radical. Perfluoroalkoxy groups are exemplified by trifluoromethoxy, pentafluoroethoxy, and the like.

The term “spirocyclyl,” as used herein, represents a C₂₋₇ alkyl diradical, both ends of which are bonded to the same carbon atom of the parent group to form a spirocyclic group, and also a C₁₋₆ heteroalkyl diradical, both ends of which are bonded to the same atom. The heteroalkyl radical forming the spirocyclyl group can containing one, two, three, or four heteroatoms independently selected from the group consisting of nitrogen, oxygen, and sulfur. In some embodiments, the spirocyclyl group includes one to seven carbons, excluding the carbon atom to which the diradical is attached. The spirocyclyl groups of the disclosure may be optionally substituted with 1, 2, 3, or 4 substituents provided herein as optional substituents for cycloalkyl and/or heterocyclyl groups.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present disclosure may exist in different tautomeric forms, all of the latter being included within the scope of the present disclosure.

The term “sulfoalkyl,” as used herein, represents an alkyl group, as defined herein, substituted by a sulfo group of—SO₃H. In some embodiments, the alkyl group can be further substituted with 1, 2, 3, or 4 substituent groups as described herein, and the sulfo group can be further substituted with one or more O-protecting groups (e.g., as described herein).

The term “sulfonyl,” as used herein, represents an —S(O)₂— group.

The term “thioalkylaryl,” as used herein, represents a chemical substituent of formula —SR, where R is an alkylaryl group. In some embodiments, the alkylaryl group can be further substituted with 1, 2, 3, or 4 substituent groups as described herein.

The term “thioalkylheterocyclyl,” as used herein, represents a chemical substituent of formula —SR, where R is an alkylheterocyclyl group. In some embodiments, the alkylheterocyclyl group can be further substituted with 1, 2, 3, or 4 substituent groups as described herein.

The term “thioalkoxy,” as used herein, represents a chemical substituent of formula —SR, where R is an alkyl group, as defined herein. In some embodiments, the alkyl group can be further substituted with 1, 2, 3, or 4 substituent groups as described herein.

The term “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form multimers (e.g., through linkage of two or more polynucleotides) or conjugates, as well as to administer a payload, as described herein. Examples of chemical groups that can be incorporated into the linker include, but are not limited to, alkyl, alkenyl, alkynyl, amido, amino, ether, thioether, ester, alkyl, heteroalkyl, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

The term “mammal” means a human or other mammal or means a human being.

The term “messenger RNA” (mRNA) refers to any polynucleotide which encodes a protein or polypeptide of interest and which is capable of being translated to produce the encoded protein or polypeptide of interest in vitro, in vivo, in situ or ex vivo.

The term “modified” refers to a changed state or structure of a molecule of the disclosure. Molecules may be modified in many ways including chemically, structurally, and functionally. In one embodiment, the UNA oligomers of the present disclosure are modified by the introduction of non-natural nucleosides and/or nucleotides, e.g., as it relates to the natural ribonucleotides A, U, G, and C.

The term “microRNAs” (miRNA) means single-stranded RNA molecules of 21-23 nucleotides in length, which regulate gene expression miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

The term “naturally occurring” means existing in nature without artificial aid.

The term “nucleotide” means natural bases (standard) and modified bases well known in the art. Such bases are generally located at the 1′ position of a nucleotide sugar moiety. Nucleotides generally comprise a base, sugar, and a phosphate group. The nucleotides can be unmodified or modified at the sugar, phosphate, and/or base moiety, (also referred to interchangeably as nucleotide analogs, modified nucleotides, non-natural nucleotides, non-standard nucleotides and other; see, for example, Usman and McSwiggen, supra; Eckstein, et al., International PCT Publication No. WO 92/07065; Usman, et al., International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include: inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine, and uracil at 1′ position or their equivalents.

The term “off target” refers to any unintended effect on any one or more target, gene, or cellular transcript.

The term “open reading frame” or “ORF” to a nucleic acid sequence (DNA or RNA) which is capable of encoding a polypeptide of interest. ORFs often begin with the start codon ATG, and end with a nonsense or termination codon or signal.

The phrase “operably linked” refers to a functional connection between two or more molecules, constructs, transcripts, entities, moieties or the like.

The term “paratope” refers to the antigen-binding site of an antibody.

The term “peptide” is less than or equal to 50 amino acids long, e.g., about 5, 10, 15, 20, 25, 30, 35, 40, 45, or 50 amino acids long.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The phrase “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington’s Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “physicochemical” means of or relating to a physical and/or chemical property.

The term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “RNA” means a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA. A ribonucleotide is a nucleotide with a hydroxyl group at the 2′ position of a β-D-ribo-furanose moiety. These terms include double-stranded RNA, single-stranded RNA, isolated RNA such as partially purified RNA, essentially pure RNA, synthetic RNA, recombinantly produced RNA, as well as modified and altered RNA that differs from naturally occurring RNA by the addition, deletion, substitution, modification, and/or alteration of one or more nucleotides. Alterations of an RNA can include addition of non-nucleotide material, such as to the end(s) of an interfering RNA or internally, for example at one or more nucleotides of an RNA nucleotides in an RNA molecule include non-standard nucleotides, such as non-naturally occurring nucleotides or chemically synthesized nucleotides or deoxynucleotides. These altered RNAs can be referred to as analogs.

The term “RNAi” means an RNA-dependent gene silencing process that is controlled by the RNA-induced silencing complex (RISC) and is initiated by short double-stranded RNA molecules in a cell, where they interact with the catalytic RISC component argonaute. When the double-stranded RNA or RNA-like iNA or siRNA is exogenous (coming from infection by a virus with an RNA genome or from transfected iNA or siRNA), the RNA or iNA is imported directly into the cytoplasm and cleaved to short fragments by the enzyme dicer. The initiating dsRNA can also be endogenous (originating in the cell), as in pre-microRNAs expressed from RNA-coding genes in the genome. The primary transcripts from such genes are first processed to form the characteristic stem-loop structure of pre-miRNA in the nucleus, then exported to the cytoplasm to be cleaved by dicer. Thus, the two dsRNA pathways, exogenous and endogenous, converge at the RISC complex. The active components of an RNA-induced silencing complex (RISC) are endonucleases called argonaute proteins, which cleave the target mRNA strand complementary to their bound siRNA or iNA. As the fragments produced by dicer are double-stranded, they could each in theory produce a functional siRNA or iNA. However, only one of the two strands, which is known as the guide strand, binds the argonaute protein and directs gene silencing. The other anti-guide strand or passenger strand is degraded during RISC activation.

The term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

The term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

The term “solvate” means a physical association of a compound of this disclosure with one or more solvent molecules. This physical association involves varying degrees of ionic and covalent bonding, including hydrogen bonding. In certain instances, the solvate will be capable of isolation, for example, when one or more solvent molecules are incorporated in the crystal lattice of the crystalline solid. “Solvate” encompasses both solution-phase and isolatable solvates. Non-limiting examples of suitable solvates include ethanolates, methanolates, and the like.

The term “split dose” is the division of single unit dose or total daily dose into two or more doses.

The term “stable” refers to a compound that is sufficiently robust to survive isolation to a useful degree of purity from a reaction mixture, and preferably capable of formulation into an efficacious therapeutic agent.

The terms “stabilize”, “stabilized,” “stabilized region,” means to make or become stable.

The term “substituted” means substitution with specified groups other than hydrogen, or with one or more groups, moieties, or radicals which can be the same or different, with each, for example, being independently selected.

The term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “total daily dose” is an amount given or prescribed in 24 hr period. It may be administered as a single unit dose.

The term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

The term “half-life” is the time required for a quantity such as nucleic acid or protein concentration or activity to fall to half of its value as measured at the beginning of a time period.

The term “in vitro” refers to events that occur in an artificial environment, e.g., in a test tube or reaction vessel, in cell culture, in a Petri dish, etc., rather than within an organism (e.g., animal, plant, or microbe).

The term “in vivo” refers to events that occur within an organism (e.g., animal, plant, or microbe or cell or tissue thereof).

The term “monomer” refers to a single unit, e.g., a single nucleic acid, which may be joined with another molecule of the same or different type to form an oligomer. In some embodiments, a monomer may be an unlocked nucleic acid, i.e., a UNA monomer.

The term “oligomer” may be used interchangeably with “polynucleotide” and refers to a molecule comprising at least two monomers and includes oligonucleotides such as DNAs and RNAs. In the case of oligomers containing RNA monomers and/or unlocked nucleic acid (UNA) monomers, the oligomers of the present disclosure may contain sequences in addition to the coding sequence (CDS). These additional sequences may be untranslated sequences, i.e., sequences which are not converted to protein by a host cell. These untranslated sequences can include a 5′ cap, a 5′ untranslated region (5′ UTR), a 3′ untranslated region (3′ UTR), and a tail region, e.g., a polyA tail region. As described in further detail herein, any of these untranslated sequences may contain one or more UNA monomers - these UNA monomers are not capable of being translated by a host cell’s machinery. In the context of the present disclosure, a “mRNA sequence,” “translatable polynucleotide,” or “translatable compound” refers to a sequence that comprises a region that is capable of being converted to a protein or a fragment thereof, such as a coding region or coding sequence of an RNA or a codon-optimized version thereof encoding a human protein.

The terms “Small interfering RNA (siRNA)” and “short interfering RNA” and “silencing RNA” mean a class of double-stranded RNA molecules, 16-40 nucleotides in length, that play a variety of roles in biology. Most notably, siRNA is involved in the RNA interference (RNAi) pathway, where it interferes with the expression of a specific gene. In addition to their role in the RNAi pathway, siRNAs also act in RNAi-related pathways, e.g., as an antiviral mechanism or in shaping the chromatin structure of a genome; the complexity of these pathways is only now being elucidated.

The terms “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

The term “translatable” may be used interchangeably with the term “expressible” and refers to the ability of polynucleotide, or a portion thereof, to be converted to a polypeptide by a host cell. As is understood in the art, translation is the process in which ribosomes in a cell’s cytoplasm create polypeptides. In translation, messenger RNA (mRNA) is decoded by tRNAs in a ribosome complex to produce a specific amino acid chain, or polypeptide. Furthermore, the term “translatable” when used in this specification in reference to an oligomer, means that at least a portion of the oligomer, e.g., the coding region of an oligomer sequence (also known as the coding sequence or CDS), is capable of being converted to a protein or a fragment thereof.

The term “translation efficiency” refers to a measure of the production of a protein or polypeptide by translation of a mRNA sequence in vitro or in vivo. [0080] This disclosure provides a range of mRNA sequence molecules, which can contain one or more UNA monomers, and a number of nucleic acid monomers, wherein the mRNA sequence can be expressible to provide a polypeptide or protein.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

### Compounds and Salts

Reference to a compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when compound of the present disclosure contain both a basic moiety, such as, but not limited to, a pyridine or imidazole, and an acidic moiety, such as, but not limited to, a carboxylic acid, zwitterions (“inner salts”) may be formed and are included within the term “salt(s)” as used herein. The salts can be pharmaceutically acceptable (i.e., non-toxic, physiologically acceptable) salts, although other salts are also useful. Salts of a compound of the present disclosure may be formed, for example, by reacting a compound of the present disclosure with an amount of acid or base, such as an equivalent amount, in a medium such as one in which the salt precipitates or in an aqueous medium followed by lyophilization.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-napthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates) undecanoates, and the like. Additionally, acids which are generally considered suitable for the formation of pharmaceutically useful salts from basic pharmaceutical compound are discussed, for example, by S. Berge et al, J. Pharmaceutical Sciences (1977) 66(1)1-19; P. Gould, International J. Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated by reference herein.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine or lysine. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

All such acid and base salts are intended to be pharmaceutically acceptable salts within the scope of the disclosure and all acid and base salts are considered equivalent to the free forms of the corresponding compound of the present disclosure for purposes of the disclosure.

Compounds disclosed herein can exist in unsolvated and solvated forms, including hydrated forms. In general, the solvated forms, with pharmaceutically acceptable solvents such as water, ethanol, and the like, are equivalent to the unsolvated forms for the purposes of this disclosure.

Also within the scope of the present disclosure are polymorphs of the compound of this disclosure (i.e., polymorphs of the compound as disclosed herein are within the scope of this disclosure).

### EXAMPLES

The present disclosure is further described in the following examples, which do not limit the scope of the disclosure described in the claims.

### Example 1: Construction of Luciferase Reporter Vectors

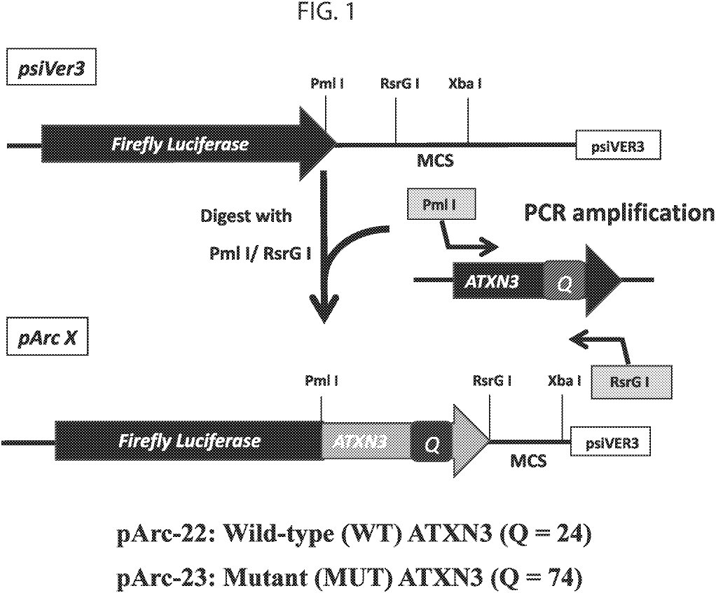

A luciferase fusion protein was designed and produced according to the following description as a tool to assess the knockdown activity of the presently disclosed unlocked nucleic acid (UNA) siRNAs. Open reading frames for human ataxin-3 with either 24 or 74 CAG repeats were PCR-amplified using genomic DNA isolated from fibroblasts in Machado-Joseph disease patient with ATXN3-specific primers containing either a Pml I or a RsrG I site and cloned in-frame into a psiVer3 vector downstream of the firefly luciferase that was constructed based on a psiCHECK™-2 vector (Promega, Madison, WI), as shown in FIG. 1. The resulting pArc vectors (i.e., pArc-22 representing wild-type (WT) ATXN-3 with only 24 CAG repeats and pArc-23 representing a mutant ATXN-3 having 74 CAG repeats) also contain a constitutively expressed Renilla luciferase gene, which served as an internal control to normalize transfection efficiency.

### Example 2: Transfection of Luciferase Reporter Vectors Into HEK293 Cells

The efficacy of the UNA siRNAs of the present disclosure was then assessed through a transfection experiment. A total of 5,000 HEK293 cells (American Type Culture Collection) was plated onto a 96-well plate one day before the transfection. The HEK293 cells were incubated at 37° C. in 100 µL of DMEM nutrient medium (Life Technologies, Carlsbad, CA) supplemented with 0.1 mM nonessential amino acids and 10% FBS (Life Technologies, Carlsbad, CA). The culture medium was changed to 90 µL of fresh medium just before the transfection. The reporter plasmid and siRNAs were co-transfected with transfection reagent. Lipofectamine™ 3000 (Life Technologies, Carlsbad, CA) was used to transfect the reporter plasmid (25 ng) and various amounts of siRNA together with P3000 into the cells according to the manufacturer’s instructions.

### Example 3: Efficacy of UNA Oligomer siRNAs Determined by Luciferase Reporter Assay

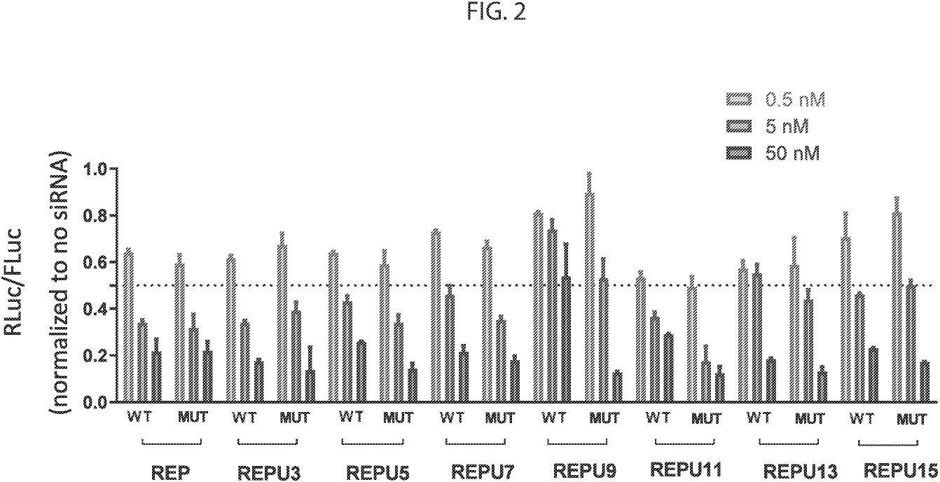

A Dual-Luciferase® Reporter Assay System (DLR assay system, Promega, Madison, WI) was used to perform dual-reporter assays on the psiCHECK2 based reporter systems. Twenty-four hours after the transfection of Example 2, the cells were washed gently with phosphate buffered saline. Then, a 40 µL aliquot of Passive Lysis Buffer (Promega, Madison, WI) was added to the cells and incubated with gentle rocking for 20 minutes at room temperature. Luciferase activities were measured using a Cytation™ 3 imaging reader (BioTek, Winooski, VT) and the effect of each of the UNA siRNAs on reporter expression was calculated based on the ratio of Firefly/Renilla to normalize cell number and transfection efficiency as shown in the results presented in FIGS. 2-5.

A reference oligomer having a non-UNA containing antisense strand was first designed, which is referred to herein as REP (Sense Strand SEQ ID NO. 2 and Antisense Strand SEQ ID NO. 4). Variants of the REP oligomer were then designed in which a single nucleotide was replaced with a UNA monomer. These UNA siRNAs were respectively referred to as REPU3 (Sense Strand SEQ ID NO. 2 and Antisense Strand SEQ ID NO. 6), REPU5 (Sense Strand SEQ ID NO. 2 and Antisense Strand SEQ ID NO. 7), REPU7 (Sense Strand SEQ ID NO. 2 and Antisense Strand SEQ ID NO. 7), REPU9 (Sense Strand SEQ ID NO. 2 and Antisense Strand SEQ ID NO. 8), REPU13 (Sense Strand SEQ ID NO. 2 and Antisense Strand SEQ ID NO. 13), and REPU15 (Sense Strand SEQ ID NO. 2 and Antisense Strand SEQ ID NO. 14). Sequences included in the various oligomers and corresponding SEQ ID NOs. are provided in Table 3. FIG. 2 shows the results of the luciferase reporter assay and the effect of these UNA siRNAs on the knockdown of the mutant ATXN3. It can be seen that generally the oligomers demonstrate a dose dependent effect with a greater knockdown activity observed as the dose increases from 0.5 nM to 5 nM then to 50 nM. The REPU9 oligomer showed great specificity toward the mutant reporter, with expression reduced to about 0.1 at the 50 nM dose while knockdown of the control at the same dose remained above 0.5. These results were then used to further design UNA siRNA oligomers for knockdown of mutant trinucleotide repeat expansion.

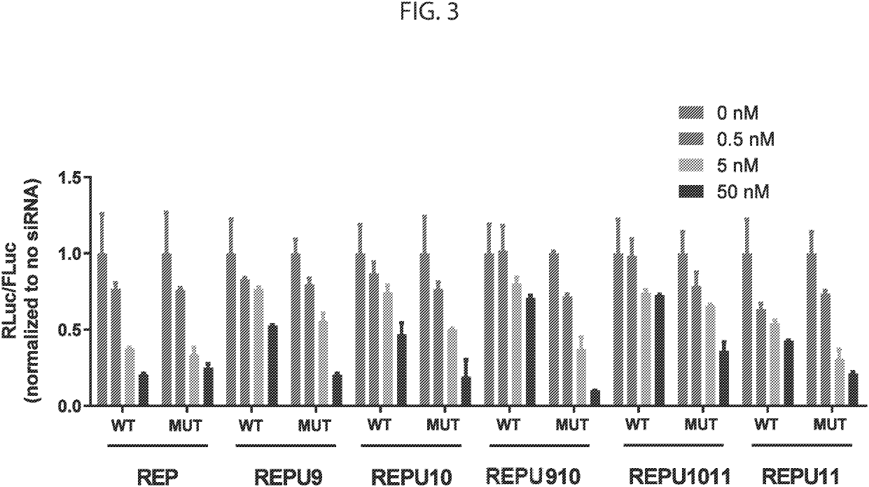

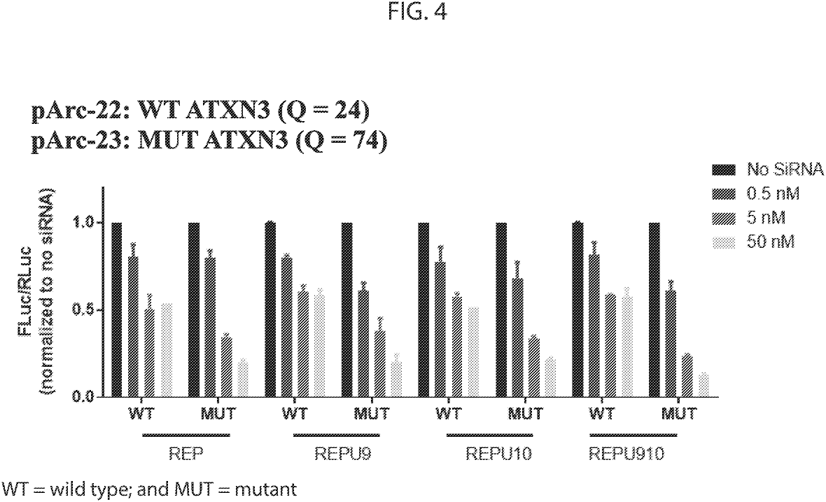

These further UNA siRNAs were designed to replace a nucleotide monomer adjacent to the UNA monomer of REPU9 or REPU11, either alone or in combination with the UNA monomer of REPU9 or REPU11. The resultant constructs were REPU10 (Sense Strand SEQ ID NO. 2 and Antisense Strand SEQ ID NO. 14), REPU910 (Sense Strand SEQ ID NO. 2 and Antisense Strand SEQ ID NO. 10), and REPU1011 (Sense Strand SEQ ID NO. 2 and Antisense Strand SEQ ID NO. 11) (sequences and corresponding SEQ ID NOs. provided in Table 3). Luciferase reporter assay results for comparing these constructs to the REP reference are shown in FIGS. 3 and 4. Each of REPU9, REPU10, and REPU11 showed good knockdown activity and selectivity for the mutant. When a combination construct REPU910 was used, the knockdown activity and selectivity were significantly improved. However, this same improvement was not observed for the construct REPU1011.

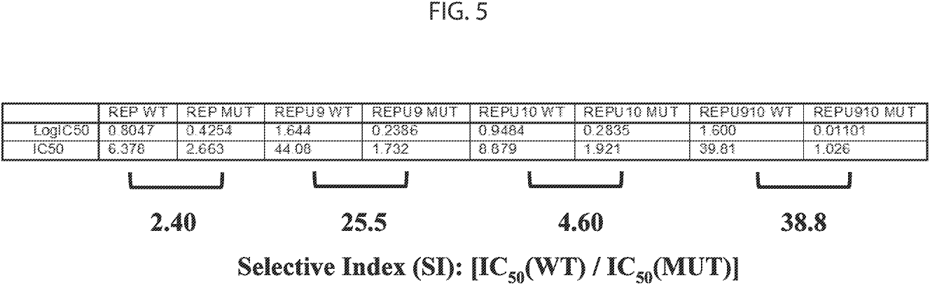

FIG. 5 further provides a table of the selective index (SI) for the constructs REP, REPU9, REPU10, and REPU910 for the mutant over the wild-type. The selective index can be thought of a ratio between activity towards one target to the activity of another. In the present experiment, the selective index for each UNA siRNA construct was calculated as the IC50 for the wild-type (WT) divided by the IC50 for the mutant (MUT). The oligomers REPU9 and REPU910 from Table 3 afforded excellent selectivity of mutant knockdown over the wild-type. As seen in FIG. 5, REPU9 showed a selective index of 25.5 and REPU910 showed a selective index of 38.8. These values far exceed the REP reference selective index of just 2.40. Moreover, REPU910 unexpectedly showed that the combination of UNA oligomers at positions 9 and 10 had a synergistic effect as the measured index of 38.8 exceeded the sum of the parts, that is REPU9 and REPU10 of 25.5 + 4.60 = 30.1.

### Example 4: Preparation of Lipid UNA siRNA Formulations

The UNA siRNA oligomers of Table 3 were lipid formulated using methods described, for example, in U.S. Application No. 16/232,212, filed on Mar. 18, 2020, the contents of which are incorporated in its entirety.

Lipid encapsulated UNA siRNA particles were prepared by mixing lipids (ionizable cationic lipid: DSPC: Cholesterol: PEG-DMG) in ethanol with UNA siRNA dissolved in citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 40-60 mM NaCl and 7-12% sucrose, pH 7.3. The formulation was concentrated followed by 0.2 µm filtration using PES filters. The UNA siRNA concentration in the formulation was then measured by Ribogreen fluorimetric assay following which the concentration was adjusted to a final desired concentration by diluting with HEPES buffer containing 40-60 mM NaCl, 7-12% sucrose, pH 7.3 containing glycerol. The final formulation was then filtered through a 0.2 µm filter and filled into glass vials, stoppered, capped and placed at -70 ± 5° C. The frozen formulations were characterized for their UNA siRNA content by HPLC or Ribogreen assay and percent encapsulation by Ribogreen assay, UNA siRNA integrity by fragment analyzer, lipid content by high performance liquid chromatography (HPLC), particle size by dynamic light scattering on a Malvern Zetasizer Nano ZS, pH and osmolality.

### Example 5: UNA siRNAs for Allele-Selective Knock Down of Androgen Receptor in SBMA

Further studies were conducted to assess the knockdown activity and selectivity of the UNA siRNAs described herein against repeat expansion mutant Spinobulbar Muscular Atrophy (SBMA) expression.

### I. Methods and Materials

### Protein Isolation and Western Blotting

Proteins analyzed in this study were isolated from fibroblasts using Cellytic™ MT Cell Lysis Reagent (Sigma-Aldrich), supplemented with Halt Protease and Phosphatase Inhibitor Cocktails (Thermo Scientific, Waltham, MA). The protein expression for the isolated protein was analyzed by western blot (WB) as described in the literature (Iida; Nature Communication, 2019). The following antibodies were used in the WB: anti-AR (androgen receptor) (1:2000; H280, Santa Cruz Biotechnology, Santa Cruz, CA), anti-GAPDH (Glyceraldehyde 3-phosphate dehydrogenase) (1:5000; 6C5, Abcam, Cambridge, MA). The density of each band in the western blot was quantitated by ImageJ software (NIH, Bethesda, MD).

### Cell Culture and Transfection

Dermal fibroblasts were collected at the biopsy stage from genetically confirmed SBMA patients and healthy control human fibroblasts were obtained from Kurabo Industries Ltd, Osaka, JP. The CAG repeat lengths for each of these sample populations were determined by PCR and Sanger sequencing. Next, the fibroblasts were maintained in Dulbecco’s Modified Eagle’s Medium (DMEM) supplemented with 10% fetal bovine serum (FBS). The cells were then plated in 6-well plates 24 hours before transfection, and the UNA siRNAs were transfected into cells with Lipofectamine™ RNAiMAX Transfection Reagent (Invitrogen), according to the manufacturer’s instructions. These transfected cells were then cultured in DMEM with 10% FBS and 50 nM dihydrotestosterone. Finally, the protein was isolated 48 hours after transfection.

### II. Results

The selective suppression of polyglutamine-expanded androgen receptor by UNA-modified siRNAs targeting CAG repeats was assessed. The UNA siRNAs tested were the REP control reference, REPU9, and REPU910. In addition, non-transfected cells (NTC) were also tested as a control. For reference, DNA sequence “CAG” encodes glutamine (symbol Gln or Q). For example, “Q48” refers to 48 copies of glutamine. In the present disclosure, the term “Q” followed by a numeral refers to the number sequential glutamine residues in the repeat domain of each fibroblast sample.

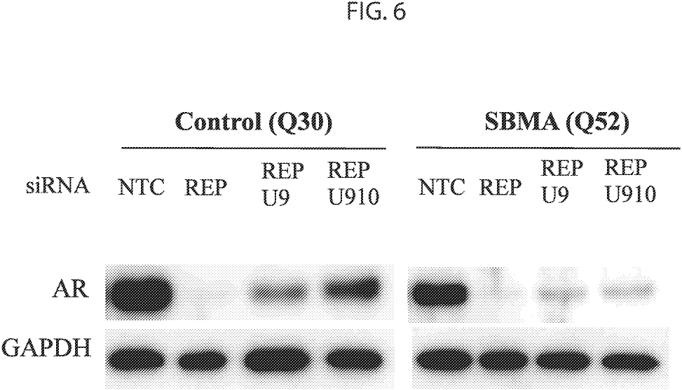

FIG. 6 shows the results of western blot analysis derived from the androgen receptor (AR) levels in healthy control (Q30) and SBMA (Q52) fibroblasts 48 hours after transfection, and these are compared with GAPDH, which serves as a positive indicator of cellular activity and that knockdown of AR is not due to toxicity from the UNA siRNAs tested. GAPDH was expressed for NTC as well as for REP, REPU9, and REPU910, indicating cells were viable throughout the experiment. For the REP reference construct, which did not include any UNA in the antisense strand, AR was knocked down for both the control Q30 and for the SBMA mutant Q52. Thus, the REP oligomer did not show selectivity for the SBMA mutant over the control.

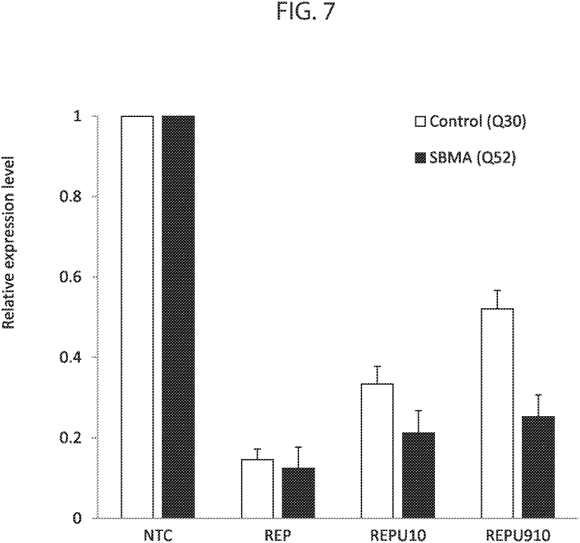

In contrast, strong expression bands for AR were observed for both REPU9 and REPU910 siRNAs for the control Q30, but these bands were substantially diminished in the mutant SBMA Q52 lanes. Thus, REPU9 and REPU910 both showed selective knockdown of SBMA while the REP construct did not. This high selectivity of knockdown effect of the UNA siRNAs on the AR expressed in fibroblasts from the SBMA patients in vitro is more clearly evident with the quantitative data shown in FIG. 7 for NTC, REP, REPU9, and REPU910 siRNAs. The bar graph shown in FIG. 7 was derived from the densitometry quantitation of AR protein expression levels obtained from WB analysis. No knockdown was observed for NTC for either the control or SBMA samples. The REP construct showed knockdown for both the control and SBMA, with relative expression levels for both samples of less than 0.2 In contrast, REPU9 showed a decrease from a relative expression level of about 0.4 for the control to about 0.2 for SBMA, and REPU910 showed a decrease from about 0.6 to about 0.2. This significant difference in knockdown activity between the control and SBMA shows that REPU9 and REPU10 have a significantly improved selectivity over the REP reference oligomer.

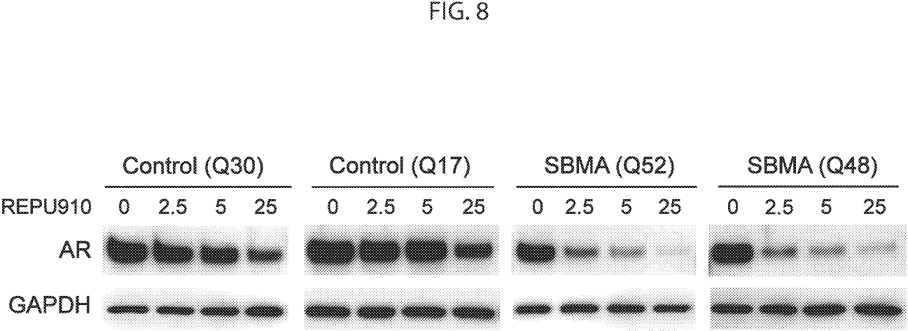

To further understand the dose response and selectivity toward differing polyglutamine repeat lengths for REPU910 siRNA, the effect of different concentrations of REPU910 (0, 2.5 nM, 5 nM, and 25 nM) on AR protein levels was measured in healthy controls (Q30 and Q17) and SBMA (Q52 and Q48) fibroblasts. FIG. 8 shows the western blot bands for AR and GAPDH control. GAPDH was expressed at all concentrations of REPU910 for both of the controls and both of the SBMA samples tested, indicating cell viability throughout the experiment. In addition, AR was expressed at all concentrations of REPU910 for both Control Q30 and Control Q17. For samples derived from SBMA patients, the intensity of the AR band decreased with increasing concentration of REPU910, thus indicating a dose-dependent effect.

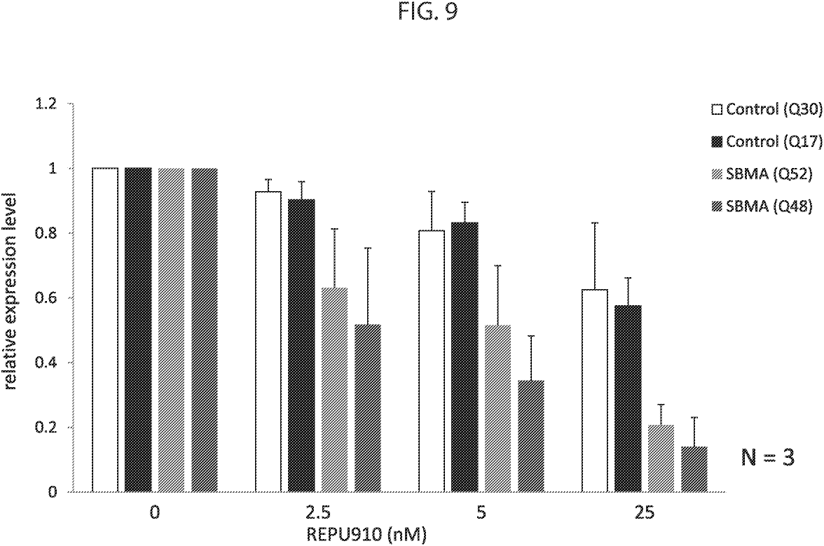

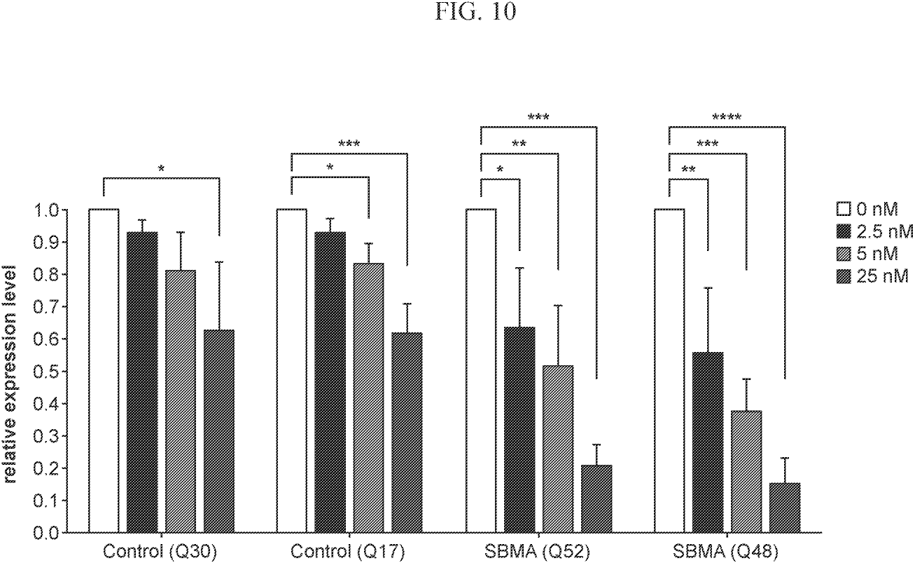

To quantitate relative expression of levels of the AR receptor for the western blot of FIG. 8, E Densitometry quantitation of the AR protein levels shown in FIG. 8 was performed, in which n = 3 (n=number of experiments); and *p<0.05, **p<0.01, ***p<0.001; and ****p<0.0001, and further using a one-way ANOVA (analysis of variance) with a post hoc Dunnett’s test. The results of these quantitated relative expression levels are depicted in the bar graphs of FIGS. 9 and 10. No knockdown was observed for the concentration of 0 nM, and expression levels generally decreased as the concentration of REPU910 increased. For each of the concentrations of 2.5 nM, 5 nM, and 25 nM, the expression levels of AR were significantly decreased for the SBMA Q52 and Q48 samples as compared to the control Q17 and Q30 samples, with values of about 0.6 for the Q17 and Q30 controls, about 0.2 for Q52 SBMA, and 0.15 for Q48 SBMA at the 25 nM concentration. These measurements confirm that REPU910 showed a high selectivity for polyglutamine expanded SBMA over the control.

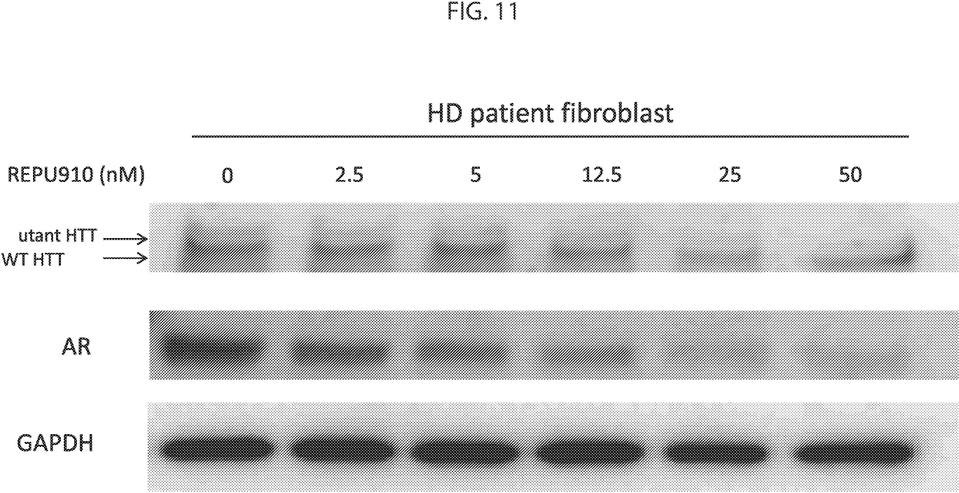

Additional dose-dependent experiments were conducted to assess the effect of REPU910 on the knockdown of AR and Huntingtin (HTT). The experiments were conducted in fibroblasts obtained from Huntington’s Disease patients, with transfections at varying concentrations of REPU910 UNA siRNA (0, 2.5, 5, 12.5, 25 and 50 nM) being performed according to the methods described above for SBMA fibroblasts. Western blot analysis was then performed using anti-HTT antibody, anti-AR antibody, and anti-GAPDH antibody. The results are shown in FIG. 11. At all concentrations of REPU910 UNA siRNA, expression of GAPDH was maintained, indicating that cells maintained their viability throughout the study. Additionally, wild-type (WT) HTT was also expressed even at concentrations of 50 nM. However, the level (band intensity) of mutant HTT and AR decreased in a dose-dependent manner, with mutant HTT bands no longer visible and AR bands almost completely knocked down at concentrations of 25 and 50 nM. Thus, REPU910 showed highly effective knockdown and selectivity of mutant HTT over the wild-type.

### Example 6

### Selective Suppression of Polyglutamine-Expanded AR by LIPID FORMULATION:REPU910 siRNA

### I. Methods and Materials

### Animals

Further experiments were conducted to assess the effect of REPU910 in vivo. The mouse protocols employed in this study were performed on transgenic mice expressing either the mutant androgen receptor (AR97Q) or the wild-type androgen receptor (AR24Q). The AR97Q and AR24Q mice were generated and maintained as described in the literature (Katsuno et al. Neuron (2002) 35:843-54). The UNA siRNA and mRNA that were used in this study were formulated into a lipid formulation as described in Example 4.

### Intracerebroventricular (ICV) Injection

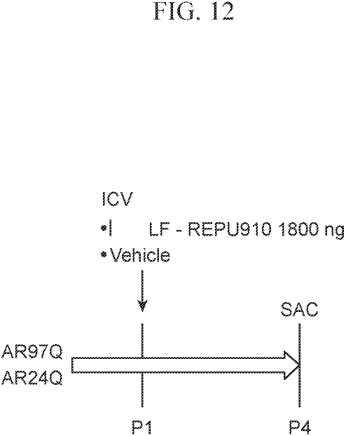

The in vivo tests of REPU910 in AR97Q and AR24Q mice were conducted using an intracerebroventricular injection (ICV) method, and FIG. 12 illustrates a scheme of the ICV experiment. This procedure has been previously described (see, for example, Sahashi et al. Genes Dev. (2012) 26:1874-84). Briefly, and in accordance with the method, at P1 neonatal mice were cryo-anesthetized on ice, and 2 µL (1800 ng) of LIPID FORMULATION-mRNA (LF-mRNA) or LIPID FORMULATION-UNA siRNA (LF-UNA siRNA) in saline containing Fast Green FCF (0.01% [w/v]; Sigma-Aldrich, St. Louis, MO) was injected into the lateral ventricle of the brain for each mouse using a 5-mL microsyringe (Hamilton Company, Reno, NV) and a 33-gauge needle. At P4, the mice were sacrificed, and their brains were dissected for further analysis.

### Protein Isolation and Western Blotting

After the mice were sacrificed, their brain regions and spinal cord were dissected and snap-frozen in powdered CO₂ in acetone. The protein fraction was isolated from mouse tissue and fibroblasts using Cellytic™ MT Cell Lysis Reagent (Sigma-Aldrich), supplemented with Halt™ Protease and Phosphatase Inhibitor Cocktails (Thermo Scientific, Waltham, MA). Next, the proteins were separated on 5-20% SDS-PAGE gels (Wako, Osaka, Japan) and the gels were then transferred to Hybond™-P membranes (GE Healthcare, Piscataway, NJ, USA). The primary antibodies used in this study were anti-AR (1:2000; H280, Santa Cruz Biotechnology, Santa Cruz, CA), anti-GAPDH (1:5000; 6C5, Abcam, Cambridge, MA), and anti-GFP (Green Fluorescent Protein) (1:1000; D5.1, Cell Signaling Technology, Beverly, MA). The density of each band was quantitated by ImageJ software (NIH, Bethesda, MD).

### II. Results

### Selective Suppression of Polyglutamine-Expanded AR by LIPID FORMULATION:REPU910 siRNA

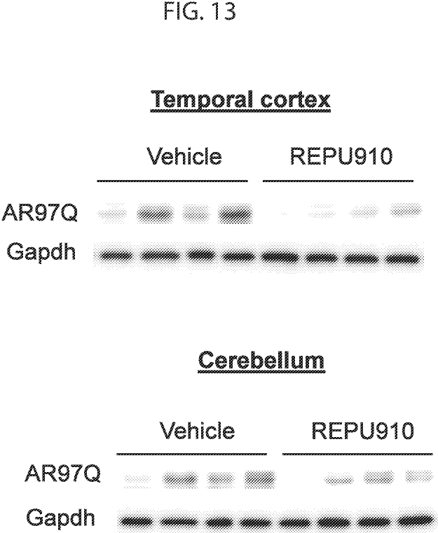

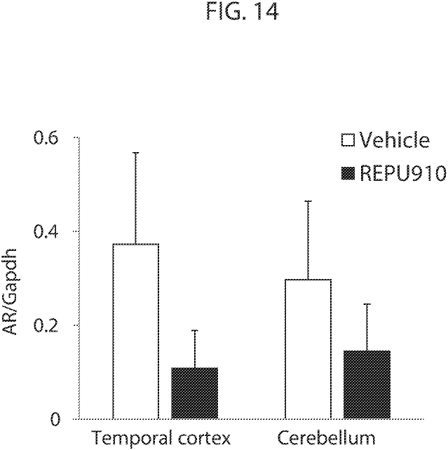

To assess the selectivity of REPU910 siRNA in vivo, transgenic mice carrying wild-type human AR (AR24Q) and mutant AR (AR97Q) were used. Vehicle (negative control) and lipid formulated REPU910 siRNA (LF-REPU910) were separately intracerebroventricularly administered in neonatal mice at P1. At P4, the mice were sacrificed and their brains were dissected. Three days after administration, AR protein levels were analyzed from tissues collected from the temporal cortex and the cerebellum. FIG. 13 shows the results of the western blot of samples from the temporal cortex and the cerebellum of AR97Q mice. The AR levels for the vehicle showed a greater intensity (establishing baseline levels) than the corresponding bands for LF-REPU910 treated samples. In addition, GAPDH levels were comparable for both vehicle and LF-REPU910 in both the temporal cortex and the cerebellum samples, indicating that LF-REPU910 knockdown of the mutant AR was not due to a toxicity effect, but rather confirming that the knockdown effect was due to RNA interference. In agreement with this observation, FIG. 14 shows densitometry quantitation of these AR97Q protein levels (n = 4, *p<0.05) normalized to GAPDH quantitation. The normalized expression levels showed about 70% suppression of mutant AR in the temporal cortex and 50% in the cerebellum of AR97Q mice that had been administered LF-REPU910.

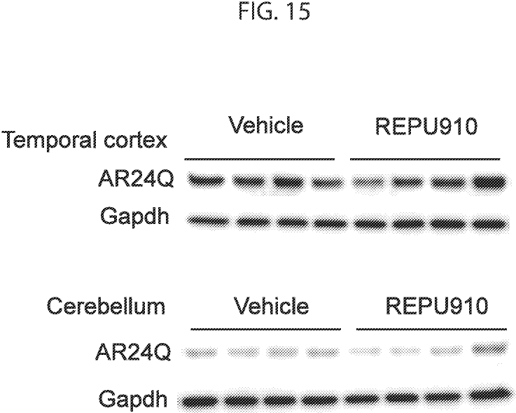

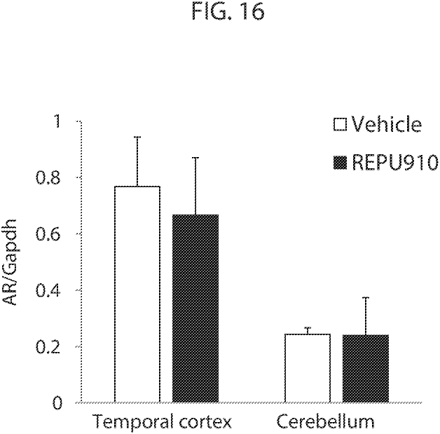

In contrast, western blot results of samples from the temporal cortex and the cerebellum of AR24Q mice showed substantially comparable expression intensity between vehicle and LF-REPU910 (FIG. 15). This low knockdown activity was confirmed by densitometry quantitation of AR24Q protein levels (normalized to GAPDH expression) for these samples (n = 4; FIG. 16). LF-REPU910 hardly suppressed wild-type AR expression in the temporal cortex and cerebellum of AR24Q mice, with levels decreasing from about 0.75 for the vehicle negative control to about 0.65 for LF-REPU910 (~13% decrease in expression) in the temporal cortex and almost no difference in expression levels observed for the cerebellum. Thus, the REPU910 oligomer showed high in vivo selectivity for the mutant AR.

### Example 7: Biodistribution Studies From Detection of ICV Injected LIPID FORMULATION-eGFP mRNA

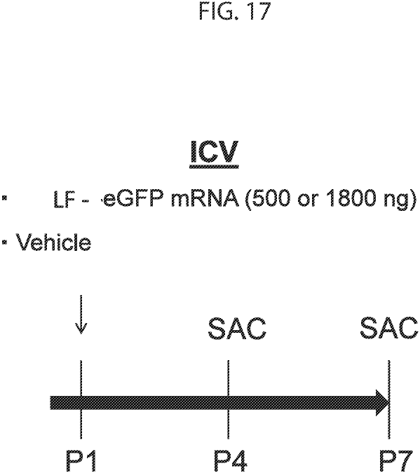

To further assess tissue uptake of lipid formulated agents, studies were designed for expressing enhanced green fluorescent protein (eGFP) in the brains of mice, which was then imaged to determine where uptake of lipid formulations occurred. FIG. 17 illustrates a scheme of the experiment. LIPID FORMULATION-eGFP mRNA (LF-eGFP mRNA) was prepared according to the method described in Example 4. The LF-eGFP mRNA particles (500 or 1800 ng) or empty vehicle (negative control) were intracerebroventricularly (ICV) injected into the lateral ventricle of P1 neonatal mice. Then, the mice were sacrificed at P4 or P7, and their brains were dissected. The P4 or P7 mouse brains were dissected in chilled phosphate-buffered saline (PBS) and imaged by fluorescent stereomicroscope (SZX16, Olympus, Tokyo, Japan). For the coronal sections, the brains were fixed in 4% paraformaldehyde for 1 hour and sectioned before the imaging experiments were carried out.

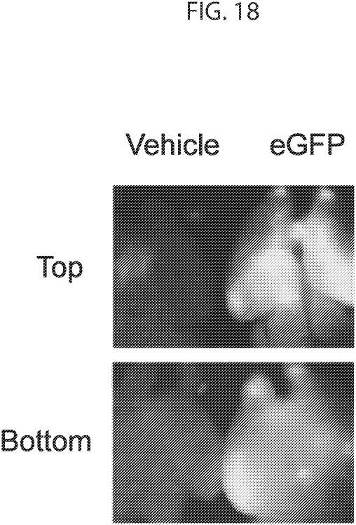

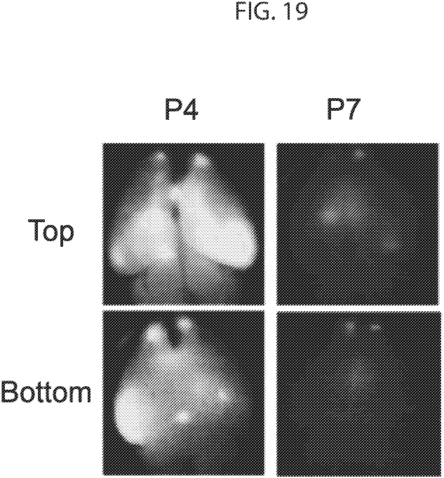

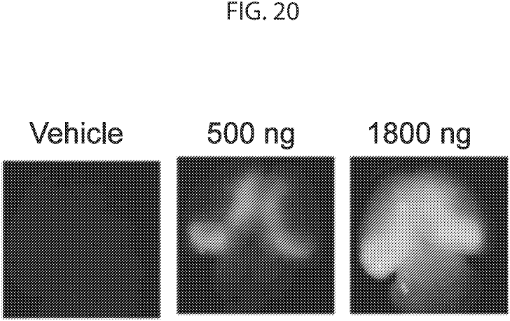

FIG. 18 shows the eGFP fluorescent images of the dissected brains, with both top and bottoms views for the vehicle negative control and for mice administered 1800 ng LF-eGFP mRNA. The high fluorescent intensity in the eGFP images as compared to the absence of detectable fluoresecence in the vehicle only negative controls indicates a high level of LF-eGFP mRNA uptake in the brains of the mice. However, at P7 the signal observed in eGFP images became weaker as compared to P4, but was still detectable (FIG. 19), showing that expression was transient. Furthermore, the eGFP signal increased in a dose-dependent manner, as seen by fluorescence imaging of tissue from vehicle negative control administered mice and samples from mice administered either 500 ng or 1800 ng LF-eGFP mRNA (FIG. 20).

### Example 8: Distribution of LIPID FORMULATION-eGFP mRNA Expression in the Central Nervous System

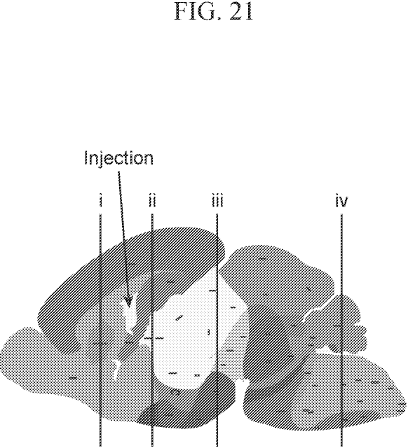

The experiments of Example 7 were extended to assess how LF-eGFP mRNA uptake is distributed in different regions of the central nervous system. FIG. 21 shows an illustration of an atlas of P4 sagittal brain, which specifically indicate the coronal sections: (i) the olfactory bulb; (ii) the lateral ventricle; (iii) the hippocampus; and (iv) the temporal cortex.

Mice were treated with 1800 ng LF-eGFP mRNA as described in Example 7, sacrificed at P4 and dissected to assess the distribution in the various regions of the brain via eGFP fluorescence imaging per the protocols described in Example 7. In addition, immunohistochemistry and western blotting studies were conducted.

For the immunohistochemistry studies, mouse brains were dissected and fixed immediately in a 10% buffered formalin solution. Sections (3 µm) were deparaffinized, heated in a microwave for 15 minutes in 10 mM citrate buffer (pH 6.0), and incubated overnight with the following primary antibody: anti-GFP (1:200; D5.1, Cell Signaling Technology). The samples were incubated with a secondary antibody labeled with a polymer as part of the Envision + system containing horseradish peroxidase (Dako Cytomation, Gostrup, Denmark). Images of IHC stained sections were photographed using an optical microscope (BX51, Olympus).

For the western blotting studies, the mice were sacrificed, and their brain regions and spinal cord were dissected and snap-frozen in powdered CO₂ in acetone. The protein fraction was isolated from mouse tissue and fibroblasts using Cellytic™ MT Cell Lysis Reagent (Sigma-Aldrich), supplemented with Halt™ Protease and Phosphatase Inhibitor Cocktails (Thermo Scientific, Waltham, MA). Next, equal amounts of protein were separated on 5-20% SDS-PAGE gels (Wako, Osaka, Japan) and then the gels were transferred to Hybond™-P membranes (GE Healthcare, Piscataway, NJ, USA). Anti-GFP antibody was used as the primary antibody (1:1000; D5.1, Cell Signaling Technology, Beverly, MA).

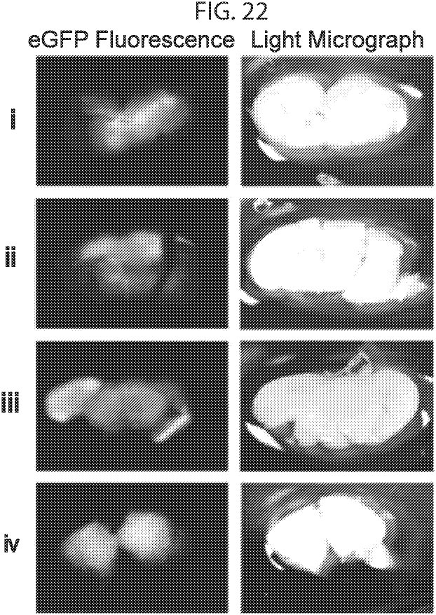

FIG. 22 shows fluorescence images and stereoscopic images (micrographs) for the dissected mouse brains. The left panels show the fluorescence images for LF-eGFP mRNA treated mice. Strong fluorescence was observed for each of (i) the olfactory bulb; (ii) the lateral ventricle; (iii) the hippocampus; and (iv) the temporal cortex. The stereoscopic images in the right panels show the full structure for each brain region and further that the uptake of LF-eGFP mRNA had a wide distribution. These images show effective delivery to the specific regions of the brain indicated above.

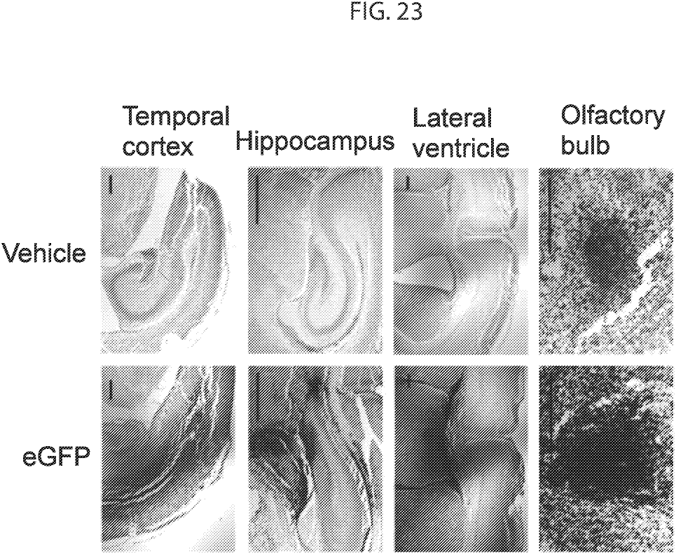

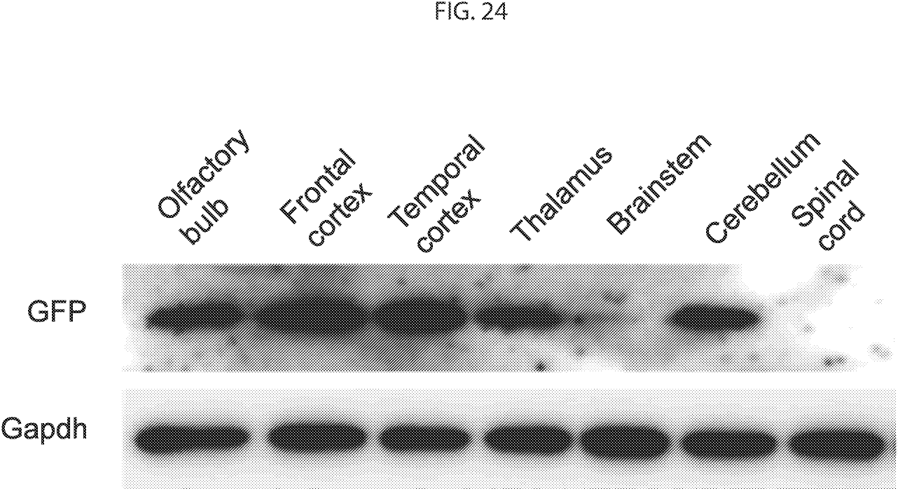

Immunohistochemistry (FIG. 23) was consistent with the results shown in FIG. 22. Significant eGFP expression was seen in micrographs of the temporal cortex, hippocampus, olfactry bulb, and lateral ventricle (FIG. 23). Finally, western blotting images are shown in FIG. 24, in which eGFP expression was low in the brainstem and spinal cord, strong in the olfactory bulb, frontal cortex, and temporal cortex, and moderate in the thalamus and brainstem.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. An oligomer comprising a sense strand and an antisense strand that mediates RNA interference against a target RNA sequence having a trinucleotide repeat expansion, wherein:
a) the antisense strand comprises a sequence having at least 80% identity to the sequence of Formula (I): rGrCrUrGrCrUrGrCX¹X²rCrUrGrCrUrGrCrUrG (I), wherein X¹ and X² are each independently selected from the group consisting of rA, rU, rG, rC, UNA-A, UNA-U, UNA-G, and UNA-C and at least one of X¹ and X² is a UNA monomer;
b) the oligomer comprises a UNA monomer at the first position at the 5′-end of the sense strand; and
c) the sense strand and the antisense strand each independently comprise 19-29 monomers.

2. The oligomer of claim 1, wherein the antisense strand comprises a sequence having at least 85% identity to the sequence of Formula (I).

3. The oligomer of any one of claims 1-2, wherein the antisense strand comprises a sequence having at least 90% identity to the sequence of Formula (I).

4. The oligomer of any one of claims 1-3, wherein the antisense strand comprises a sequence having at least 95% identity to the sequence of Formula (I).

5. The oligomer of any one of claims 1-4, wherein the antisense strand comprises a sequence having at least 99% identity to the sequence of Formula (I).

6. The oligomer of any one of claims 1-5, wherein the sense strand and the antisense strand each comprise deoxy T at the first position and the second position from the 3′ end.

7. The oligomer of any one of claims 1-6, wherein the oligomer further comprises one or more nucleic acid monomer analogs selected from the group consisting of locked nucleic acids, phosphorothioates, phosphoramidates, methyl phosphonates, chiral-methyl phosphonates, 2′-O-methyl ribonucleotides and peptide-nucleic acids.

8. The oligomer of any one of claims 1-7, wherein X¹ or X² is UNA-A.

9. The oligomer of any one of claims 1-7, wherein X¹ orX² is UNA-G.

10. The oligomer of any one of claims 1-7, wherein X¹ or X² is UNA-U.

11. The oligomer of any one of claims 1-7, wherein X¹ or X² is UNA-C.

12. The oligomer of any one of claims 1-11, wherein the UNA monomer at the first position at the 5′-end of the sense strand is UNA-A, UNA-U, UNA-G, or UNA-C.

13. The oligomer of any one of claims 1-7, wherein the UNA monomer at the first position at the 5′-end of the sense strand is UNA-C.

14. The oligomer of any one of claims 1-13, wherein the oligomer has one or two overhangs.

15. The oligomer of any one of claims 1-13, wherein the oligomer has at least one 3′-overhang.

16. The oligomer of any one of claims 1-13, wherein the oligomer has at least one 5′-overhang.

17. The oligomer of any one of claims 1-13, wherein the oligomer has at least one blunt end.

18. The oligomer of any one of claims 1-17, wherein the oligomer has reduced off-target effects as compared to an identical oligonucleotide with natural RNA monomers.

19. The oligomer of any one of claims 1-18, wherein the oligomer has increased or prolonged potency for gene silencing as compared to an identical oligonucleotide with natural RNA monomers.

20. The oligomer of any one of claims 1-19, wherein the sense and antisense strands are connected and form a duplex region with a loop at one end.

21. The oligomer of any one of claims 1-20, wherein the oligomer selectively inhibits mutant gene expression over wild-type gene expression.

22. The oligomer of any one of claims 1-21, wherein the oligomer selectively inhibits mutant gene expression versus wild-type gene expression by a factor of at least 5-fold.

23. The oligomer of any one of claims 1-22, wherein the sense strand comprises a sequence of SEQ ID NO: 2.

24. The oligomer of any one of claims 1-23, wherein the antisense strand comprises a sequence selected from SEQ ID NOs: 8-10.

25. The oligomer of any one of claims 1-23, wherein the antisense strand comprises a sequence of SEQ ID NO: 10.

26. The oligomer of any one of claims 1-22, wherein the sense strand comprises a sequence of SEQ ID NO: 2 and the antisense strand comprises a sequence of SEQ ID NO: 10.

27. The oligomer of any one of claims 1-26, wherein the oligomer is a conjugated oligomer of Formula (II) 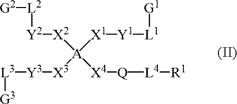
or a pharmaceutically acceptable salt or solvate thereof, wherein
A is a carbon;
X¹, X² and X³ of Formula (II) are each independently selected from the group consisting of C₁₋C₁₀ alkyl, —(CH₂)ₘ—O—(CH₂)ₙ— and —(CH₂)ₘ—N—(CH₂)ₙ—, wherein n is 1-36 and m is 1-30;
Y¹, ^{y2} and Y³ are each independently selected from the group consisting of —NHC(O)—C(O)NH—, —OC(O)—, —C(O)O—, —SC(O)—, —C(O)S— and P(Z)(OH)O₂, wherein Z is O or S;
L¹, L² and L³ are each independently selected from the group consisting of a C₁-C₁₀ alkyl, —(CH₂)ₑ—O—(CH₂)f—, —(CH₂)ₑ—S—(CH₂)f—, —(CH₂)ₑ—S(O)₂—(CH₂)f—, —(CH₂)ₑ—N—(CH₂)_{f}— and —(CH₂—CH₂—O)ₖ(CH₂)₂—, wherein e is 1-10, f is 1-16; and k is 1-20;
G¹, G² and G³ are each independently selected from the group consisting of a monosaccharide, a monosaccharide derivative, a vitamin, a polyol, a polysialic acid and a polysialic acid derivative;
X⁴ is selected from the group consisting of
(a) —(CH₂)_{g}—O—(CH₂)ₕ— or —(CH₂)_{g}—N—(CH₂)ₕ—, wherein g is 1-30 and h is 1-36,
(b) an amino acid, and
(c) —NHC(O)R², wherein R² is C₁-C₁₀ alkyl, a carbocycle, a heterocyclyl, a heteroaryl, a C₁-C₁₀ alkyl-carbocycle, a C₁-C₁₀ alkyl-heterocyclyl or a C₁-C₁₀ alkyl-heteroaryl, and wherein R² is optionally substituted;
Q is absent, alkylamino, —C(O)—(CH₂)ᵢ—, —(CH₂)ᵢ—O—(CH₂)ⱼ—, —(CH₂)ᵢ—NR³—(CH₂)ⱼ—, —(CH₂)ᵢ—S—S—(CH₂)ⱼ—, —(CH₂)ᵢ—S—(CH₂)ⱼ—, —(CH₂)ᵢ—S(O)₂—(CH₂)ⱼ—, —(CH₂)ᵢ—NHC(O)—(CH₂)ⱼ—, —(CH₂)ᵢ—C(O)NH—(CH₂)ⱼ—, —(CH₂)ᵢ—SC(O)—(CH₂)ⱼ—, or —(CH₂)ᵢ—C(O)S—(CH₂)ⱼ—, wherein i is 1-30; j is 1-36; and R³ is hydrogen or an alkyl;
L⁴ is absent, —C(O)O—, —C(O)NH—, a phosphate, C₁-C₁₀ alkyl-phosphate, C₂-C₁₀ alkenylphosphate, a phosphorothioate, C₁-C₁₀ alkyl-phosphorothioate, C₂-C₁₀ alkenylphosphorothioate, a boranophospate, a C₁-C₁₀ alkyl-boranophospate, a C₂-C₁₀ alkenylboranophospate, —C(O)NH—C₁—Cioalkyl-phosphate, —C(O)NH—Cz—C₁₀alkenyl-phosphate, —C(O)O—C₁-C₁₀alkyl-phosphate, —C(O)O—C₂-C₁₀alkenyl-phosphate, —C(O)NH—C₁-C₁₀alkylphosphorothioate, —C(O)NH—C₂—Cioalkenyl-phosphorothioate, —C(O)O—C₁-C₁₀alkylphosphorothioate, —C(O)O—C₂-C₁₀alkenyl-phosphorothioate, —C(O)—NH—C₁-C₁₀alkylboranophospate, —C(O)—NH—C₂-C₁₀alkenyl-boranophospate, —C(O)O—C₁-C₁₀alkyl-boranophospate or —C(O)O—C₂-C₁₀alkenyl-boranophospate; and
R¹ is an oligomer of any one of claims 1-26 conjugated at the R¹ position at its 5′ end or its 3′ end.

28. The oligomer of claim 27, wherein G¹, G² and G³ are each independently selected from folic acid, ribose, retinol, niacin, riboflavin, biotin, glucose, mannose, fucose, sucrose, lactose, mannose-6-phosphate, N-acetylgalactosamine, N-acetylglucosamine, a sialic acid, a sialic acid derivative, allose, altrose, arabinose, cladinose, erythrose, erythrulose, fructose, D-fucitol, L-fucitol, fucosamine, fucose, fuculose, galactosamine, D-galactosaminitol, galactose, glucosamine, glucosaminitol, glucose-6 phosphate, gulose glyceraldehyde, L-glycero-D-mannosheptose, glycerol, glycerone, gulose, idose, lyxose, mannosamine, psicose, quinovose, quinovosamine, rhamnitol, rhamnosamine, rhamnose, ribulose, sedoheptulose, sorbose, tagatose, talose, threose, xylose and xylulose.

29. The oligomer of claim 27 or 28, wherein G¹, G² and G³ are each N-acetylgalactosamine.

30. The oligomer of any one of claims 27 to 29, wherein X⁴ is selected from the group consisting of 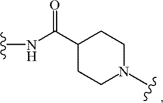 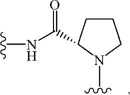 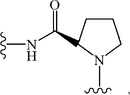 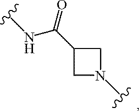 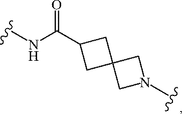 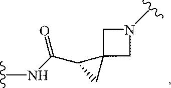 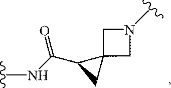 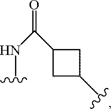 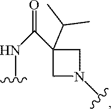 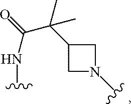 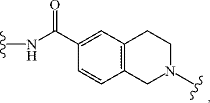 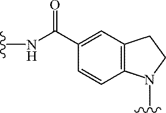 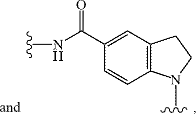
wherein X
⁴ is optionally substituted.

31. The oligomer of any one of claims 27 to 30, wherein X⁴ is 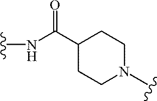
.

32. The oligomer of any one of claims 27 to 31, having the formula: 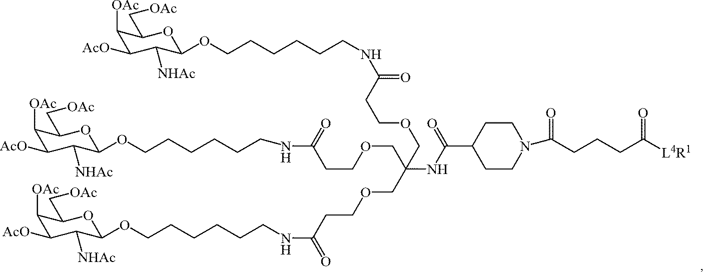
wherein R
¹ is an oligomer of any one of claims 1-26.

33. A compound selected from the group consisting of 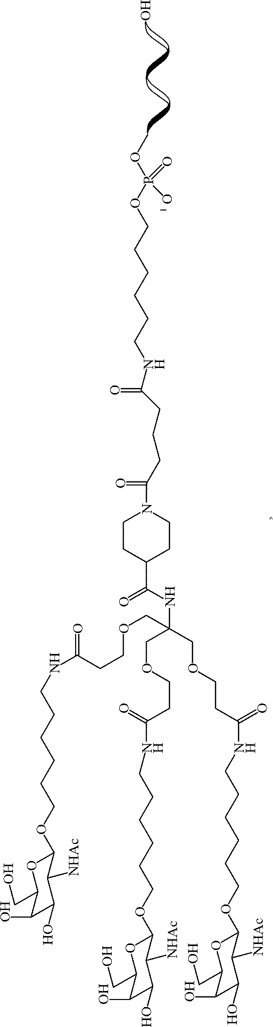 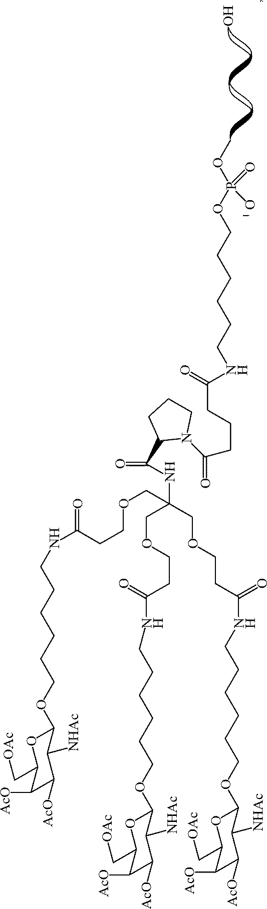 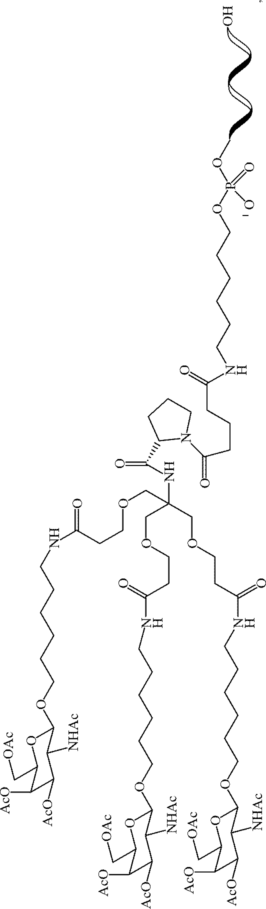 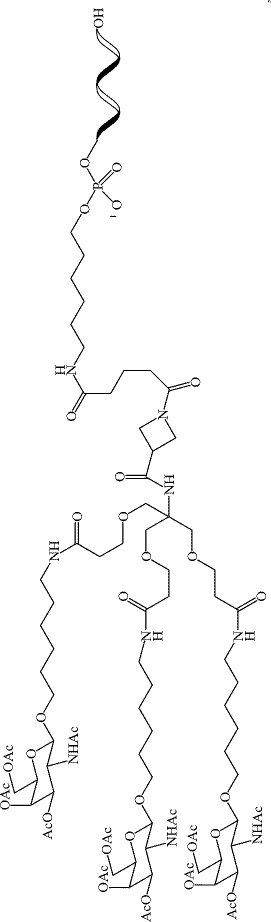 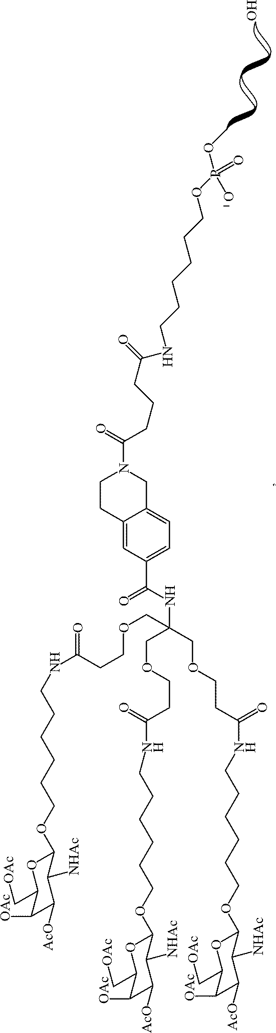 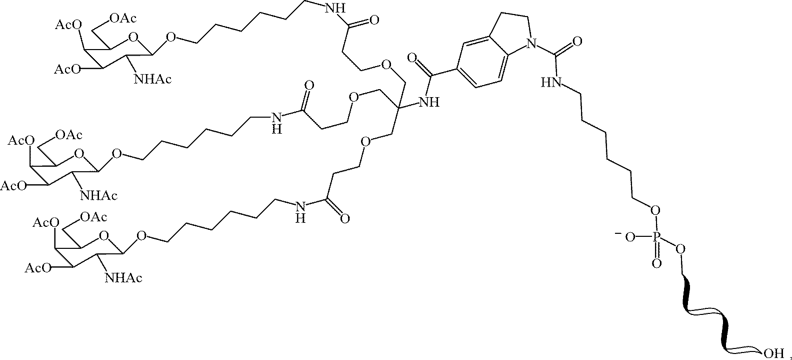
and 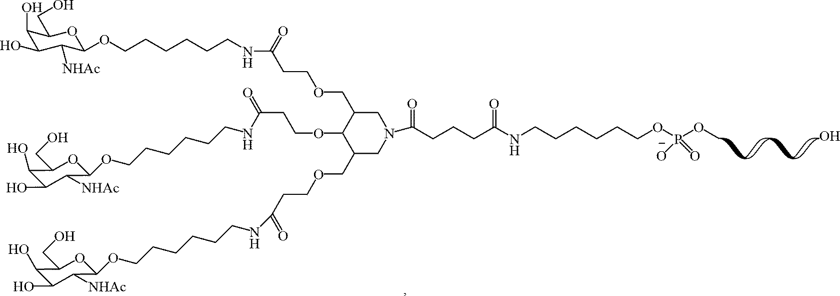
wherein 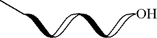
is an oligomer of any one of
claims 1-26 conjugated at its 5′ end or its 3′ end.

34. A compound having a formula selected from: 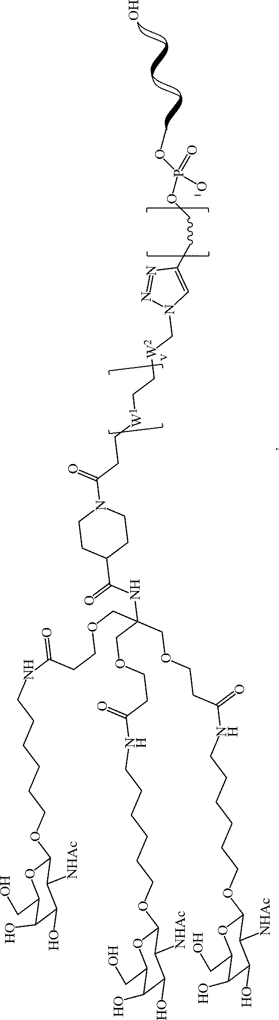 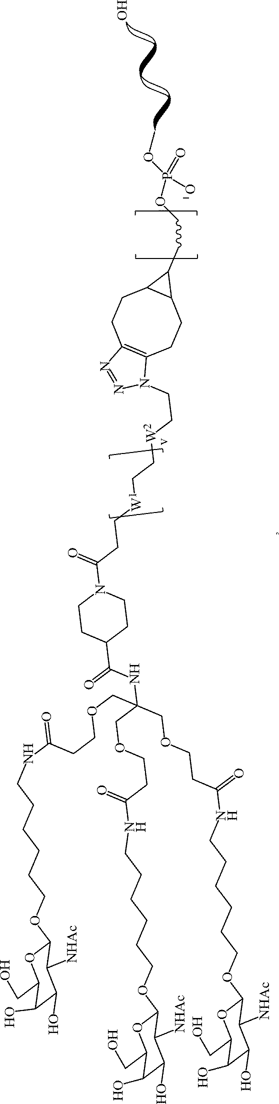 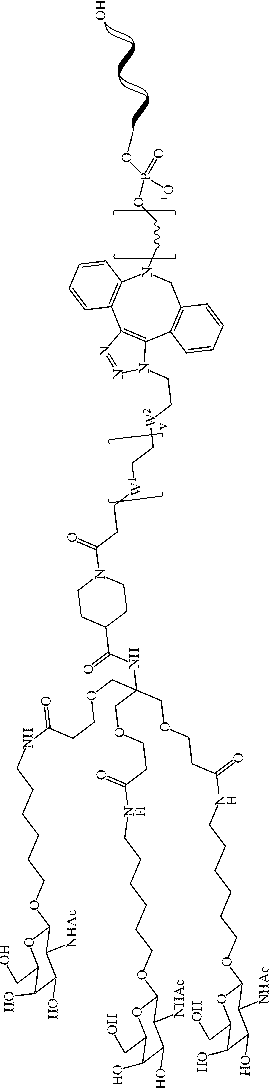
or 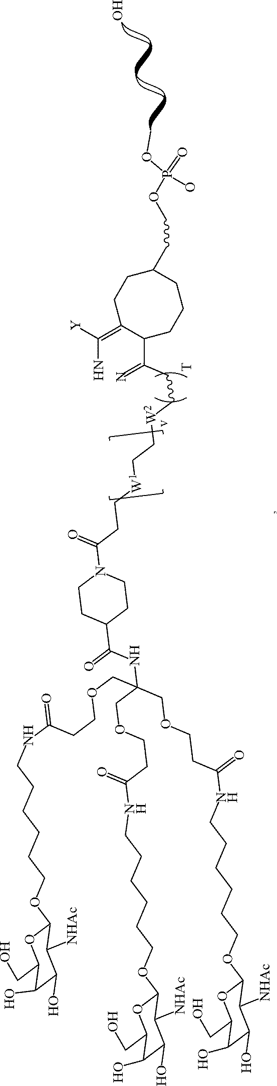
wherein 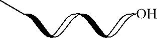
is an oligomer of any one of
claims 1-26 conjugated at its 5′ end or its 3′ end, and 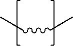
is C
₁-C₁₀ alkyl or C₂-C₁₀ alkenyl.

35. The compound of claim 34, wherein the compound is 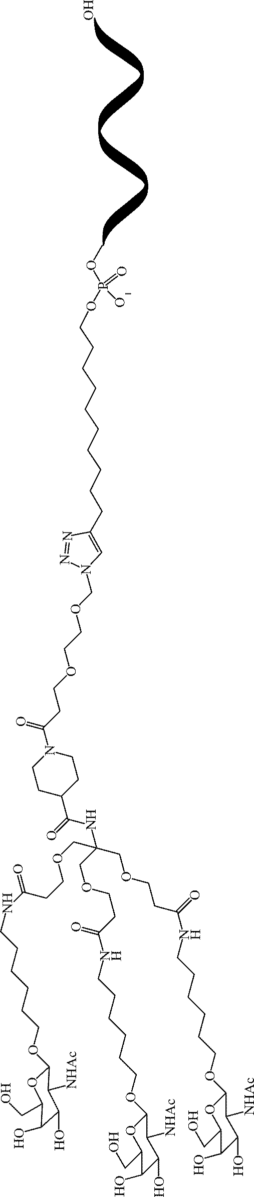 [SMILES: O ; COP(=O)([O-])OCCCCCCCCCCc1cn(COCCOCCC(=O)N2CCC(C(=O)NC(COCCC(=O)NCCCCCCOC3OC(CO)C(O)C(O)C3NC(C)=O)(COCCC(=O)NCCCCCCOC3OC(CO)C(O)C(O)C3NC(C)=O)COCCC(=O)NCCCCCCOC3OC(CO)C(O)C(O)C3NC(C)=O)CC2)nn1] 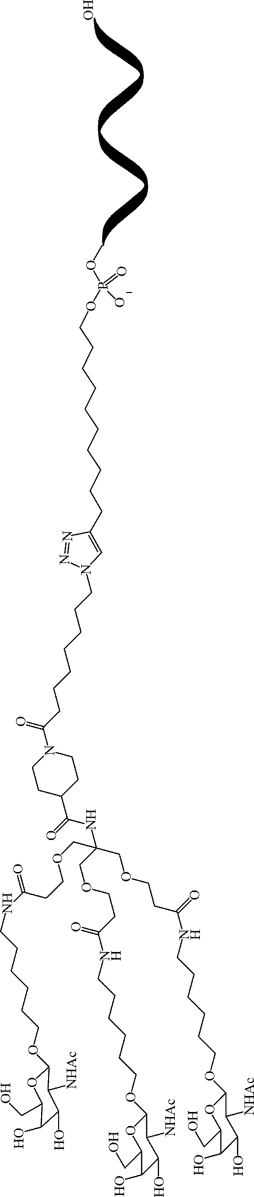 [SMILES: O ; COP(=O)([O-])OCCCCCCCCCCc1cn(CCCCCCCC(=O)N2CCC(C(=O)NC(COCCC(=O)NCCCCCCOC3OC(CO)C(O)C(O)C3NC(C)=O)(COCCC(=O)NCCCCCCOC3OC(CO)C(O)C(O)C3NC(C)=O)COCCC(=O)NCCCCCCOC3OC(CO)C(O)C(O)C3NC(C)=O)CC2)nn1] 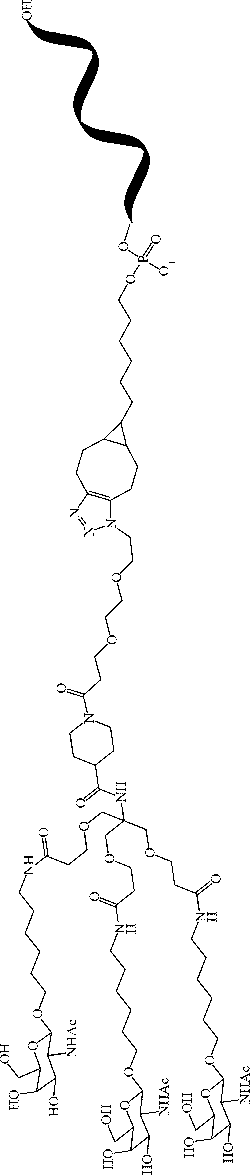 [SMILES: O ; COP(=O)([O-])OCCCCCCC1C2CCc3nnn(CCOCCOCCC(=O)N4CCC(C(=O)NC(COCCC(=O)NCCCCCCOC5OC(CO)C(O)C(O)C5NC(C)=O)(COCCC(=O)NCCCCCCOC5OC(CO)C(O)C(O)C5NC(C)=O)COCCC(=O)NCCCCCCOC5OC(CO)C(O)C(O)C5NC(C)=O)CC4)c3CCC12] 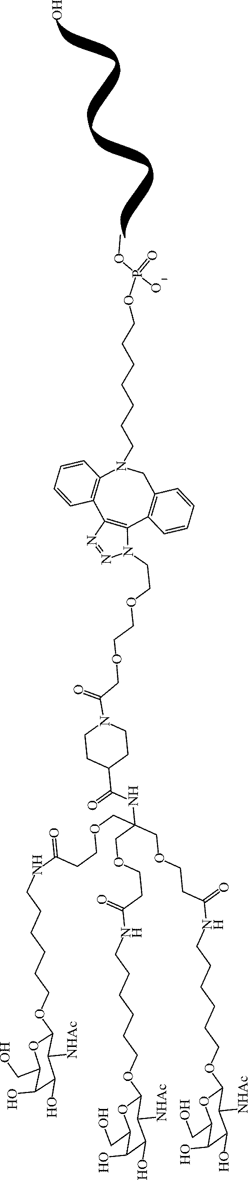 [SMILES: O ; COP(=O)([O-])OCCCCCCCN1Cc2ccccc2-c2c(nnn2CCOCCOCC(=O)N2CCC(C(=O)NC(COCCC(=O)NCCCCCCOC3OC(CO)C(O)C(O)C3NC(C)=O)(COCCC(=O)NCCCCCCOC3OC(CO)C(O)C(O)C3NC(C)=O)COCCC(=O)NCCCCCCOC3OC(CO)C(O)C(O)C3NC(C)=O)CC2)-c2ccccc21]
or 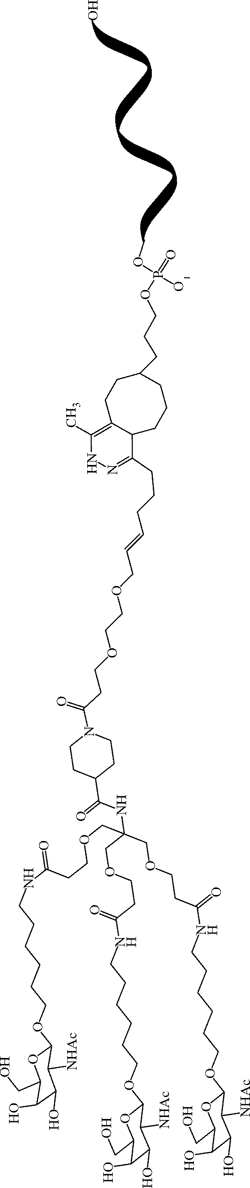 [SMILES: O ; COP(=O)([O-])OCCCC1CCCC2C(CCC/C=C/COCCOCCC(=O)N3CCC(C(=O)NC(COCCC(=O)NCCCCCCOC4OC(CO)C(O)C(O)C4NC(C)=O)(COCCC(=O)NCCCCCCOC4OC(CO)C(O)C(O)C4NC(C)=O)COCCC(=O)NCCCCCCOC4OC(CO)C(O)C(O)C4NC(C)=O)CC3)=NNC(C)=C2CC1]
.

36. A pharmaceutical composition comprising an oligomer of any one of claims 1-35 and a pharmaceutically acceptable carrier.

37. A pharmaceutical composition comprising an oligomer of any one of claims 1-35, and a lipid of Formula (V) 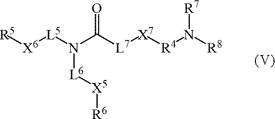
or a pharmaceutically acceptable salt or solvate thereof, wherein
R⁵ and R⁶ are each independently selected from the group consisting of a linear or branched C₁₋C₃₁ alkyl, C₂₋C₃₁ alkenyl, C₂₋C₃₁ alkynyl and cholesteryl;
L⁵ and L⁶ are each independently selected from the group consisting of a linear C₁-C₂₀ alkyl and C₂-C₂₀ alkenyl;
X⁵ is —C(O)O— or —OC(O)—;
X⁶ is —C(O)O— or —OC(O)—;
X⁷ is S or O;
L⁷ is absent or lower alkyl;
R⁴ is a linear or branched C₁₋C₆ alkyl; and
R⁷ and R⁸ are each independently selected from the group consisting of a hydrogen and a linear or branched C₁₋C₆ alkyl.

38. The pharmaceutical composition of claim 37, wherein X⁷ is S.

39. A pharmaceutical composition comprising an oligomer of any one of claims 1-35 and a lipid selected from the group consisting of 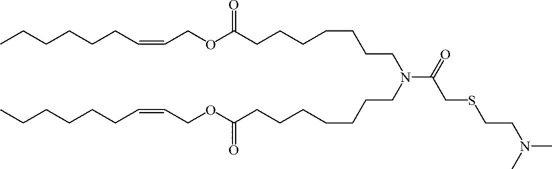 [SMILES: CCCCCC/C=C\COC(=O)CCCCCCCN(CCCCCCCC(=O)OC/C=C\CCCCCC)C(=O)CSCCN(C)C] 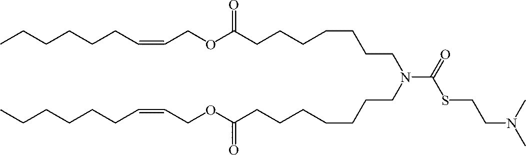 [SMILES: CCCCCC/C=C\COC(=O)CCCCCCCN(CCCCCCCC(=O)OC/C=C\CCCCCC)C(=O)SCCN(C)C] 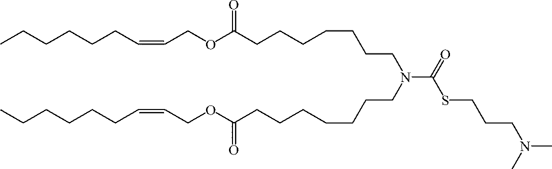 [SMILES: CCCCCC/C=C\COC(=O)CCCCCCCN(CCCCCCCC(=O)OC/C=C\CCCCCC)C(=O)SCCCN(C)C] 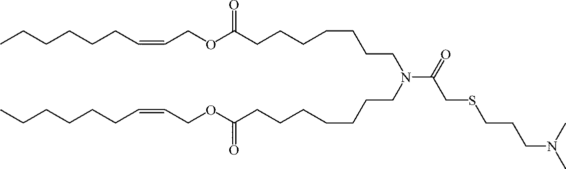 [SMILES: CCCCCC/C=C\COC(=O)CCCCCCCN(CCCCCCCC(=O)OC/C=C\CCCCCC)C(=O)CSCCCN(C)C] 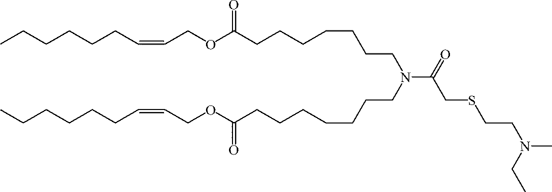 [SMILES: CCCCCC/C=C\COC(=O)CCCCCCCN(CCCCCCCC(=O)OC/C=C\CCCCCC)C(=O)CSCCN(C)CC] 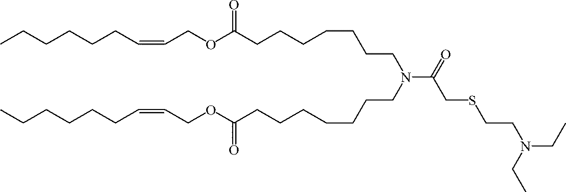 [SMILES: CCCCCC/C=C\COC(=O)CCCCCCCN(CCCCCCCC(=O)OC/C=C\CCCCCC)C(=O)CSCCN(CC)CC] 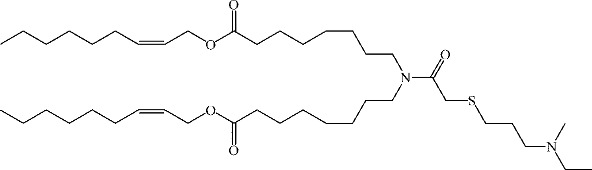 [SMILES: CCCCCC/C=C\COC(=O)CCCCCCCN(CCCCCCCC(=O)OC/C=C\CCCCCC)C(=O)CSCCCN(C)CC] 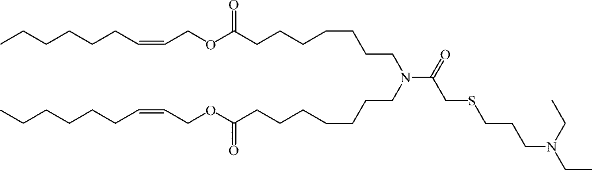 [SMILES: CCCCCC/C=C\COC(=O)CCCCCCCN(CCCCCCCC(=O)OC/C=C\CCCCCC)C(=O)CSCCCN(CC)CC] 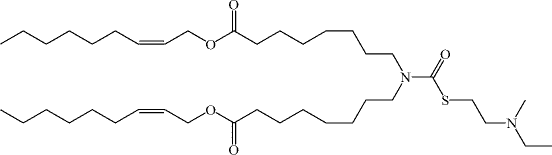 [SMILES: CCCCCC/C=C\COC(=O)CCCCCCCN(CCCCCCCC(=O)OC/C=C\CCCCCC)C(=O)SCCN(C)CC] 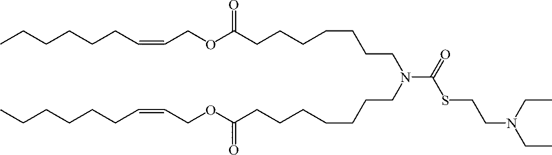 [SMILES: CCCCCC/C=C\COC(=O)CCCCCCCN(CCCCCCCC(=O)OC/C=C\CCCCCC)C(=O)SCCN(CC)CC] 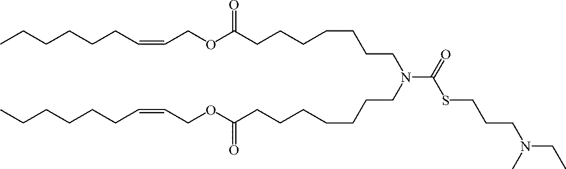 [SMILES: CCCCCC/C=C\COC(=O)CCCCCCCN(CCCCCCCC(=O)OC/C=C\CCCCCC)C(=O)SCCCN(C)CC] 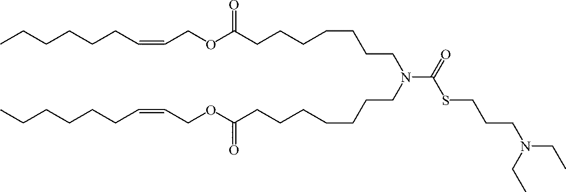 [SMILES: CCCCCC/C=C\COC(=O)CCCCCCCN(CCCCCCCC(=O)OC/C=C\CCCCCC)C(=O)SCCCN(CC)CC] 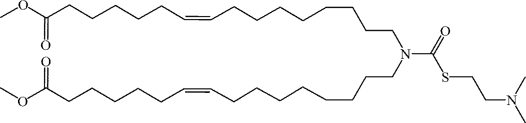 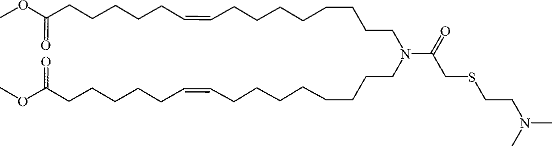 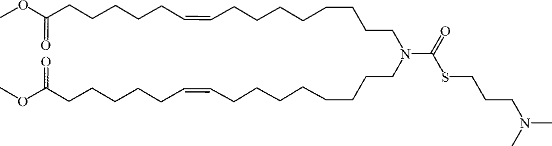 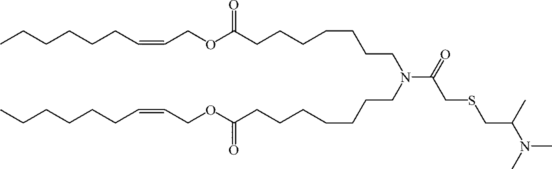 [SMILES: CCCCCC/C=C\COC(=O)CCCCCCCN(CCCCCCCC(=O)OC/C=C\CCCCCC)C(=O)CSCC(C)N(C)C] 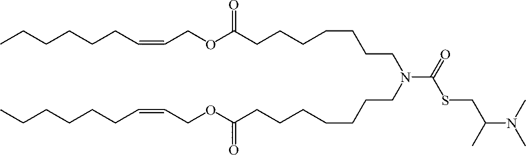 [SMILES: CCCCCC/C=C\COC(=O)CCCCCCCN(CCCCCCCC(=O)OC/C=C\CCCCCC)C(=O)SCC(C)N(C)C] 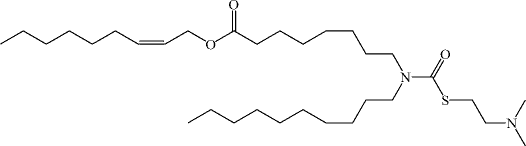 [SMILES: CCCCCC/C=C\COC(=O)CCCCCCCN(CCCCCCCCCC)C(=O)SCCN(C)C] 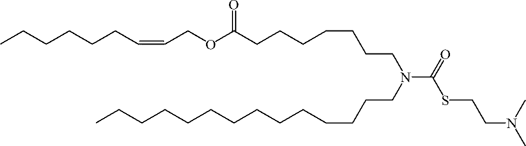 [SMILES: CCCCCC/C=C\COC(=O)CCCCCCCN(CCCCCCCCCCCCCC)C(=O)SCCN(C)C] 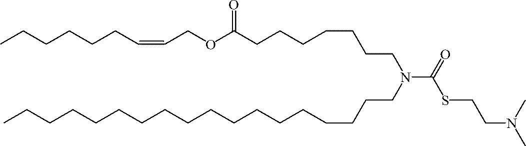 [SMILES: CCCCCC/C=C\COC(=O)CCCCCCCN(CCCCCCCCCCCCCCCCCC)C(=O)SCCN(C)C] 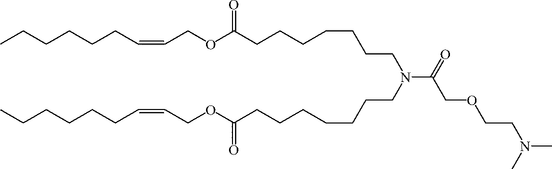 [SMILES: CCCCCC/C=C\COC(=O)CCCCCCCN(CCCCCCCC(=O)OC/C=C\CCCCCC)C(=O)COCCN(C)C] 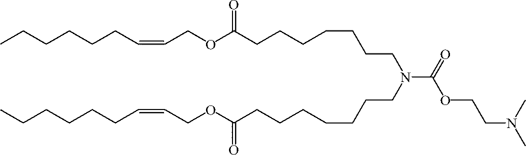 [SMILES: CCCCCC/C=C\COC(=O)CCCCCCCN(CCCCCCCC(=O)OC/C=C\CCCCCC)C(=O)OCCN(C)C] 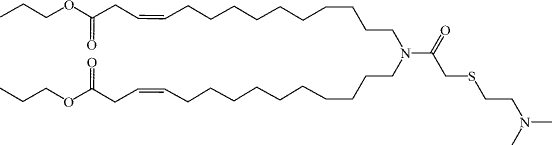 [SMILES: CCCOC(=O)C/C=C\CCCCCCCCCCN(CCCCCCCCCC/C=C\CC(=O)OCCC)C(=O)CSCCN(C)C] 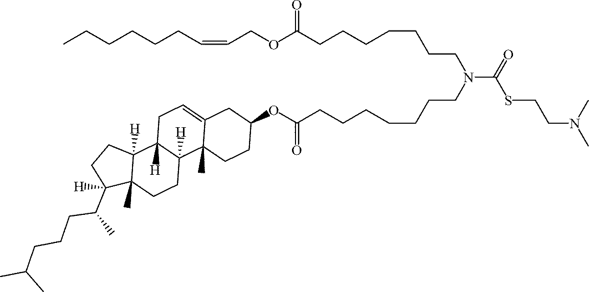 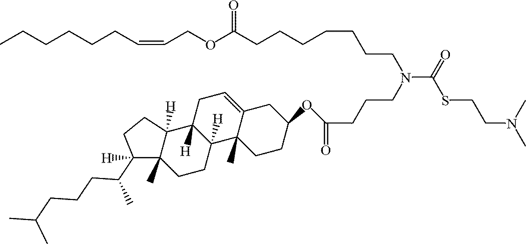 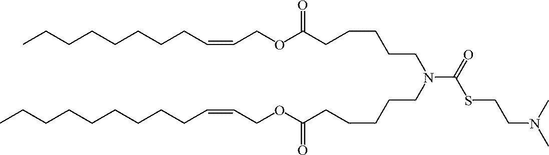 [SMILES: CCCCCCCC/C=C\COC(=O)CCCCCN(CCCCCC(=O)OC/C=C\CCCCCCCCC)C(=O)SCCN(C)C] 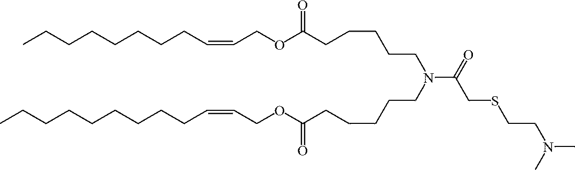 [SMILES: CCCCCCCC/C=C\COC(=O)CCCCCN(CCCCCC(=O)OC/C=C\CCCCCCCCC)C(=O)CSCCN(C)C] 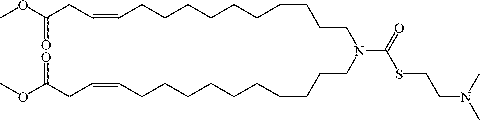 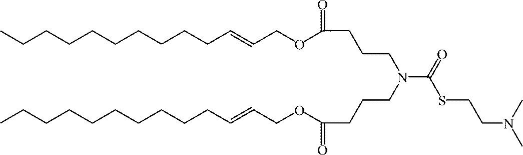 [SMILES: CCCCCCCCCC/C=C/COC(=O)CCCN(CCCC(=O)OC/C=C/CCCCCCCCCC)C(=O)SCCN(C)C] 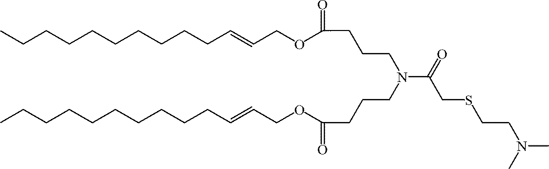 [SMILES: CCCCCCCCCC/C=C/COC(=O)CCCN(CCCC(=O)OC/C=C/CCCCCCCCCC)C(=O)CSCCN(C)C] 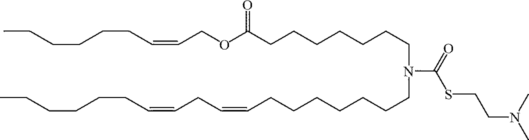 [SMILES: CCCCCC/C=C\C/C=C\CCCCCCCN(CCCCCCCC(=O)OC/C=C\CCCCCC)C(=O)SCCN(C)C] 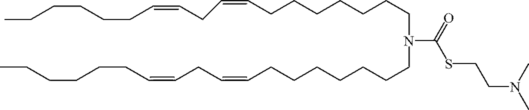 [SMILES: CCCCCC/C=C\C/C=C\CCCCCCCN(CCCCCCC/C=C\C/C=C\CCCCCC)C(=O)SCCN(C)C] 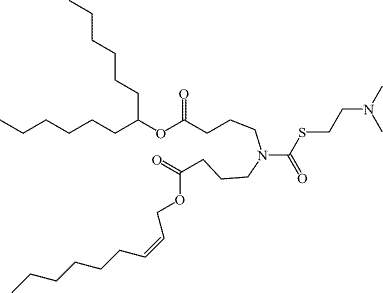 [SMILES: CCCCCC/C=C\COC(=O)CCCN(CCCC(=O)OC(CCCCCC)CCCCCC)C(=O)SCCN(C)C] 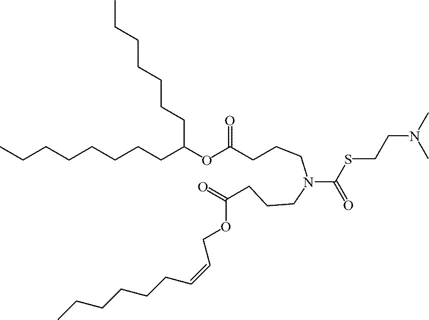 [SMILES: CCCCCC/C=C\COC(=O)CCCN(CCCC(=O)OC(CCCCCCC)CCCCCCCC)C(=O)SCCN(C)C] 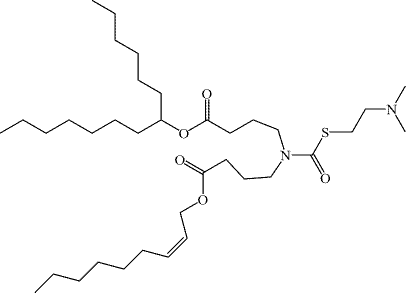 [SMILES: CCCCCC/C=C\COC(=O)CCCN(CCCC(=O)OC(CCCCCC)CCCCCCC)C(=O)SCCN(C)C] 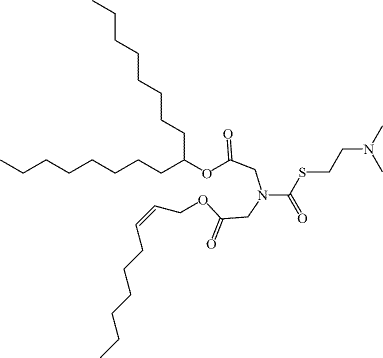 [SMILES: CCCCCC/C=C\COC(=O)CN(CC(=O)OC(CCCCCCCC)CCCCCCCC)C(=O)SCCN(C)C] 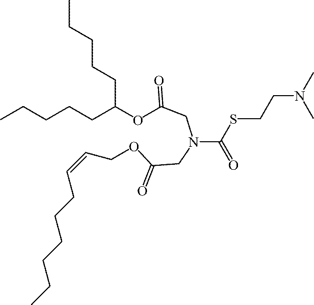 [SMILES: CCCCCC/C=C\COC(=O)CN(CC(=O)OC(CCCCC)CCCCC)C(=O)SCCN(C)C] 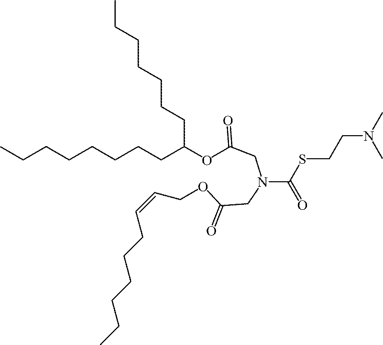 [SMILES: CCCCCC/C=C\COC(=O)CN(CC(=O)OC(CCCCCCC)CCCCCCCC)C(=O)SCCN(C)C] 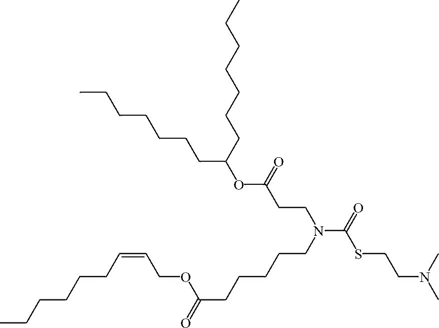 [SMILES: CCCCCC/C=C\COC(=O)CCCCCN(CCC(=O)OC(CCCCCCC)CCCCCCC)C(=O)SCCN(C)C] 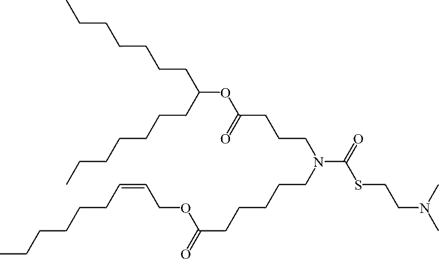 [SMILES: CCCCCC/C=C\COC(=O)CCCCCN(CCCC(=O)OC(CCCCCCC)CCCCCCC)C(=O)SCCN(C)C] 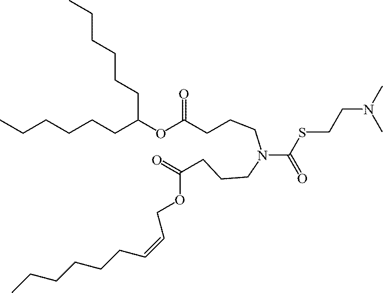 [SMILES: CCCCCC/C=C\COC(=O)CCCN(CCCC(=O)OC(CCCCCC)CCCCCC)C(=O)SCCN(C)C] 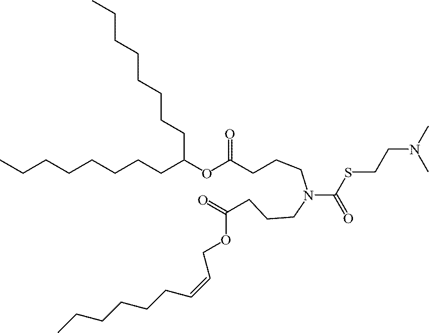 [SMILES: CCCCCC/C=C\COC(=O)CCCN(CCCC(=O)OC(CCCCCCCC)CCCCCCCC)C(=O)SCCN(C)C] 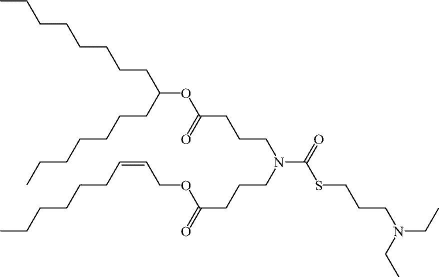 [SMILES: CCCCCC/C=C\COC(=O)CCCN(CCCC(=O)OC(CCCCCCC)CCCCCCCC)C(=O)SCCCN(CC)CC] 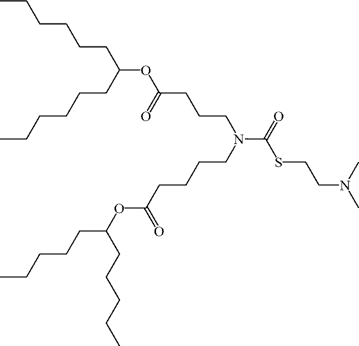 [SMILES: CCCCCCC(CCCCCC)OC(=O)CCCN(CCCCC(=O)OC(CCCCC)CCCCC)C(=O)SCCN(C)C] 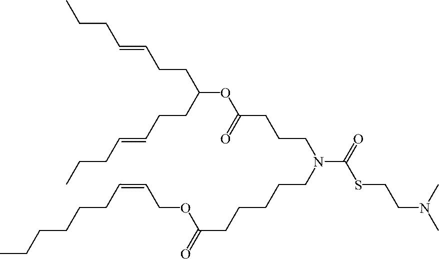 [SMILES: CCC/C=C/CCC(CC/C=C/CCC)OC(=O)CCCN(CCCCCC(=O)OC/C=C\CCCCCC)C(=O)SCCN(C)C] 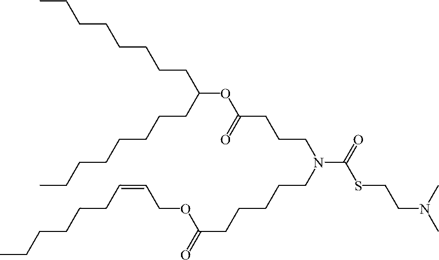 [SMILES: CCCCCC/C=C\COC(=O)CCCCCN(CCCC(=O)OC(CCCCCCCC)CCCCCCCC)C(=O)SCCN(C)C] 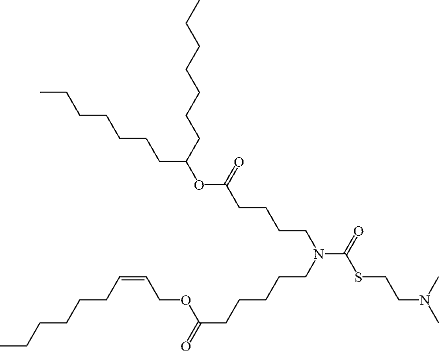 [SMILES: CCCCCC/C=C\COC(=O)CCCCCN(CCCCC(=O)OC(CCCCCCC)CCCCCCC)C(=O)SCCN(C)C] 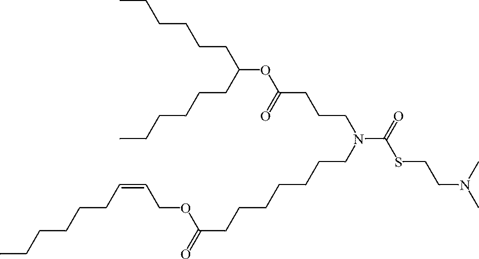 [SMILES: CCCCCC/C=C\COC(=O)CCCCCCCN(CCCC(=O)OC(CCCCCC)CCCCCC)C(=O)SCCN(C)C] 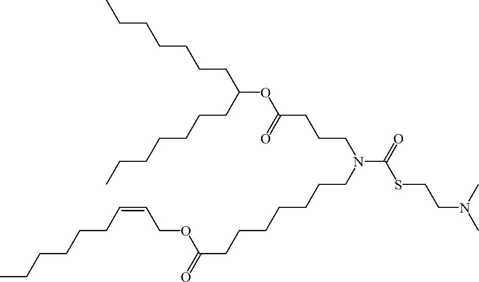 [SMILES: CCCCCC/C=C\COC(=O)CCCCCCCN(CCCC(=O)OC(CCCCCCC)CCCCCCC)C(=O)SCCN(C)C] 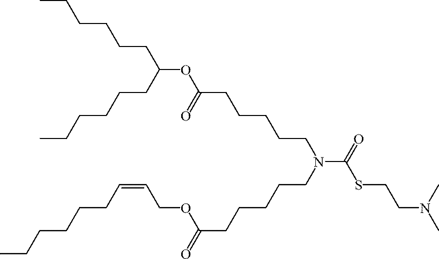 [SMILES: CCCCCC/C=C\COC(=O)CCCCCN(CCCCCC(=O)OC(CCCCCC)CCCCCC)C(=O)SCCN(C)C] 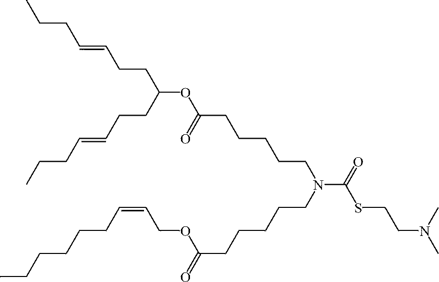 [SMILES: CCC/C=C/CCC(CC/C=C/CCC)OC(=O)CCCCCN(CCCCCC(=O)OC/C=C\CCCCCC)C(=O)SCCN(C)C] 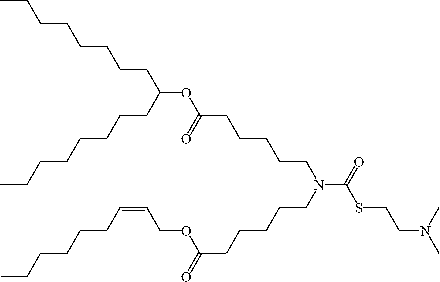 [SMILES: CCCCCC/C=C\COC(=O)CCCCCN(CCCCCC(=O)OC(CCCCCCCC)CCCCCCCC)C(=O)SCCN(C)C] 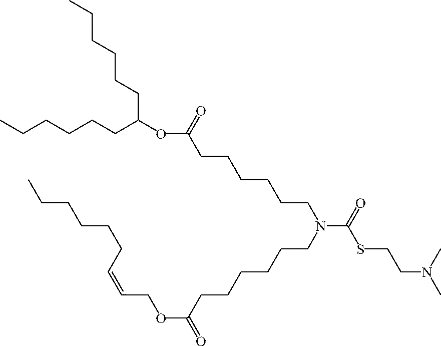 [SMILES: CCCCCC/C=C\COC(=O)CCCCCCN(CCCCCCC(=O)OC(CCCCCC)CCCCCC)C(=O)SCCN(C)C] 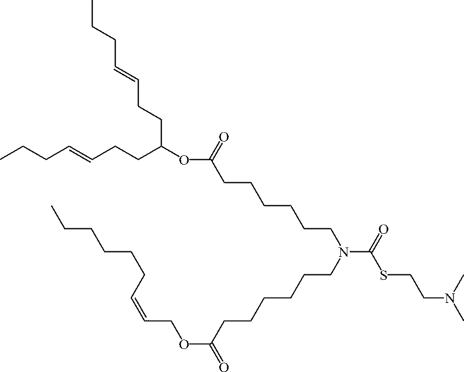 [SMILES: CCC/C=C/CCC(CC/C=C/CCC)OC(=O)CCCCCCN(CCCCCCC(=O)OC/C=C\CCCCCC)C(=O)SCCN(C)C] 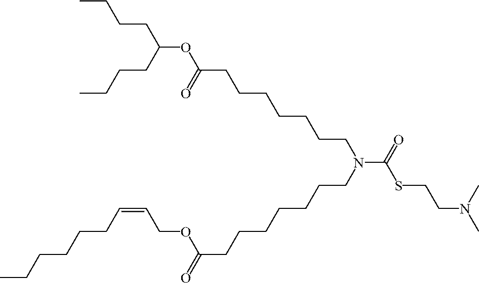 [SMILES: CCCCCC/C=C\COC(=O)CCCCCCCN(CCCCCCCC(=O)OC(CCCC)CCCC)C(=O)SCCN(C)C] 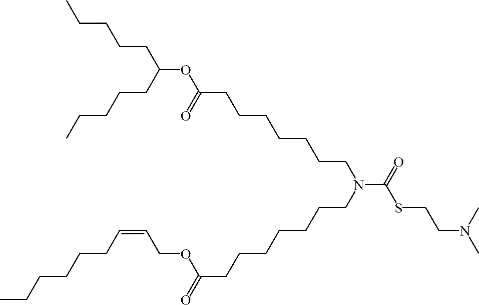 [SMILES: CCCCCC/C=C\COC(=O)CCCCCCCN(CCCCCCCC(=O)OC(CCCCC)CCCCC)C(=O)SCCN(C)C] 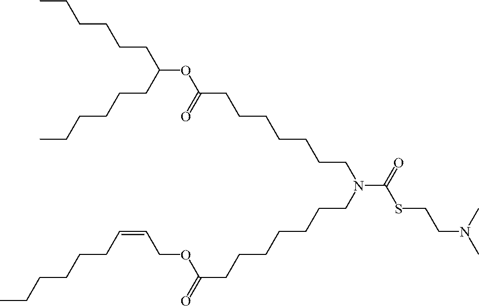 [SMILES: CCCCCC/C=C\COC(=O)CCCCCCCN(CCCCCCCC(=O)OC(CCCCCC)CCCCCC)C(=O)SCCN(C)C] 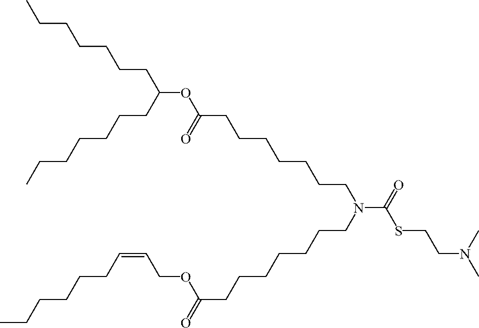 [SMILES: CCCCCC/C=C\COC(=O)CCCCCCCN(CCCCCCCC(=O)OC(CCCCCCC)CCCCCCC)C(=O)SCCN(C)C] 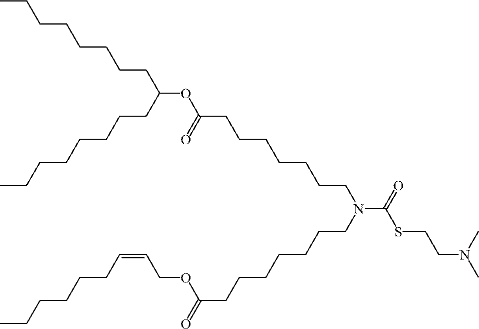 [SMILES: CCCCCC/C=C\COC(=O)CCCCCCCN(CCCCCCCC(=O)OC(CCCCCCCC)CCCCCCCC)C(=O)SCCN(C)C] 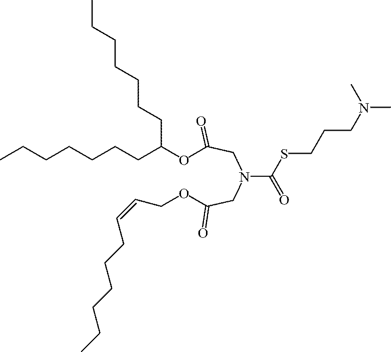 [SMILES: CCCCCC/C=C\COC(=O)CN(CC(=O)OC(CCCCCCC)CCCCCCC)C(=O)SCCCN(C)C] 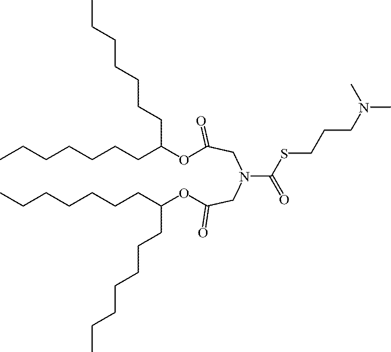 [SMILES: CCCCCCCC(CCCCCCC)OC(=O)CN(CC(=O)OC(CCCCCCC)CCCCCCC)C(=O)SCCCN(C)C] 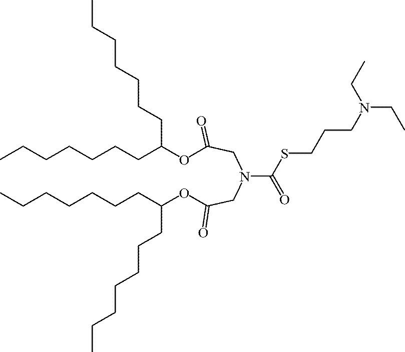 [SMILES: CCCCCCCC(CCCCCCC)OC(=O)CN(CC(=O)OC(CCCCCCC)CCCCCCC)C(=O)SCCCN(CC)CC] 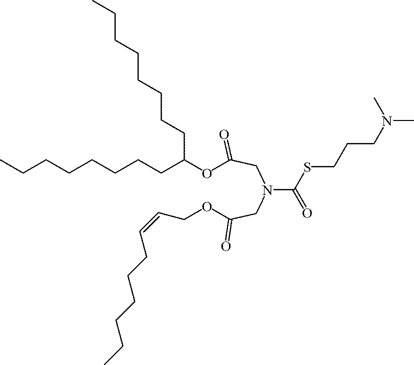 [SMILES: CCCCCC/C=C\COC(=O)CN(CC(=O)OC(CCCCCCCC)CCCCCCCC)C(=O)SCCCN(C)C] 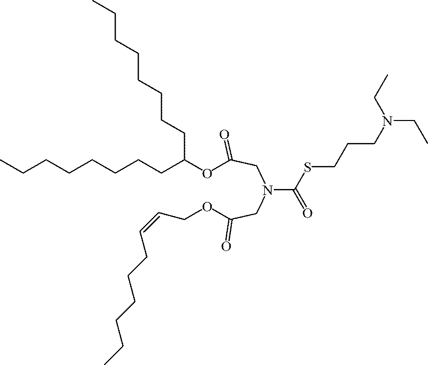 [SMILES: CCCCCC/C=C\COC(=O)CN(CC(=O)OC(CCCCCCCC)CCCCCCCC)C(=O)SCCCN(CC)CC] 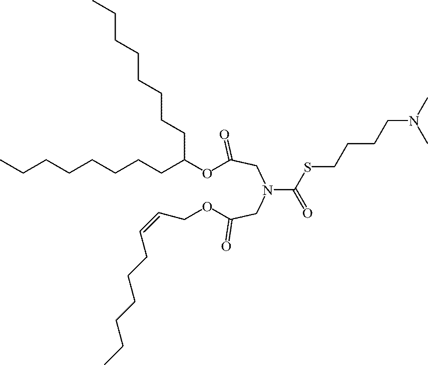 [SMILES: CCCCCC/C=C\COC(=O)CN(CC(=O)OC(CCCCCCCC)CCCCCCCC)C(=O)SCCCCN(C)C] 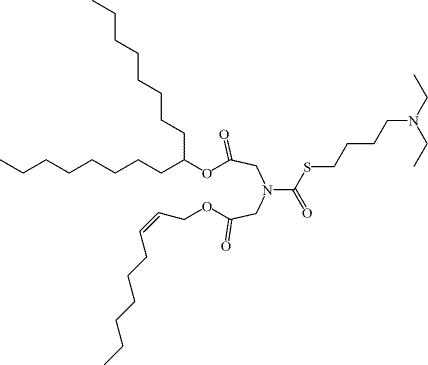 [SMILES: CCCCCC/C=C\COC(=O)CN(CC(=O)OC(CCCCCCCC)CCCCCCCC)C(=O)SCCCCN(CC)CC] 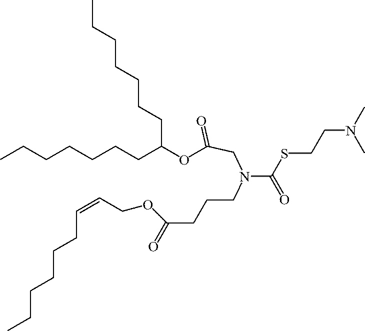 [SMILES: CCCCCC/C=C\COC(=O)CCCN(CC(=O)OC(CCCCCCC)CCCCCCC)C(=O)SCCN(C)C] 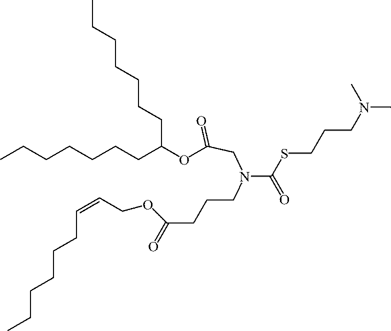 [SMILES: CCCCCC/C=C\COC(=O)CCCN(CC(=O)OC(CCCCCCC)CCCCCCC)C(=O)SCCCN(C)C] 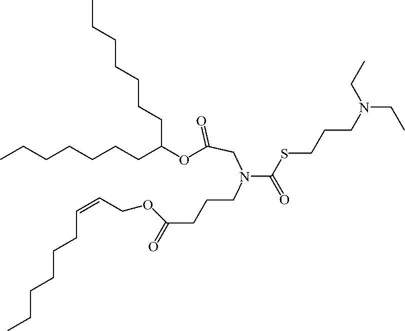 [SMILES: CCCCCC/C=C\COC(=O)CCCN(CC(=O)OC(CCCCCCC)CCCCCCC)C(=O)SCCCN(CC)CC] 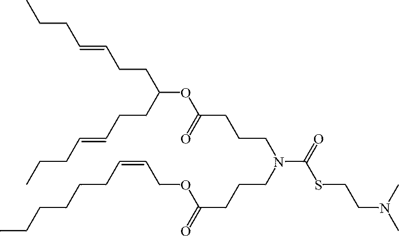 [SMILES: CCC/C=C/CCC(CC/C=C/CCC)OC(=O)CCCN(CCCC(=O)OC/C=C\CCCCCC)C(=O)SCCN(C)C] 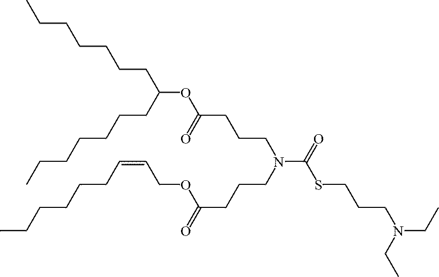 [SMILES: CCCCCC/C=C\COC(=O)CCCN(CCCC(=O)OC(CCCCCCC)CCCCCCC)C(=O)SCCCN(CC)CC] 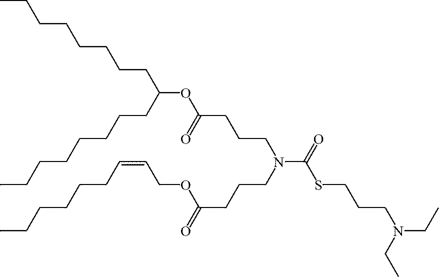 [SMILES: CCCCCC/C=C\COC(=O)CCCN(CCCC(=O)OC(CCCCCCCC)CCCCCCCC)C(=O)SCCCN(CC)CC] 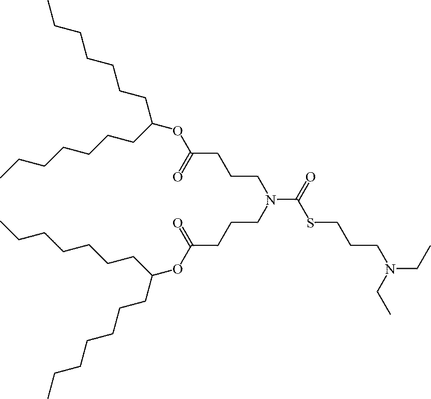 [SMILES: CCCCCCCC(CCCCCCC)OC(=O)CCCN(CCCC(=O)OC(CCCCCCC)CCCCCCC)C(=O)SCCCN(CC)CC] 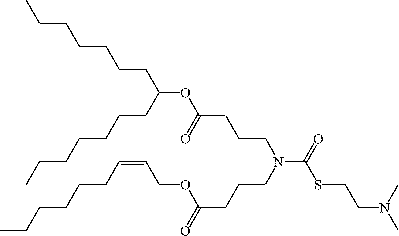 [SMILES: CCCCCC/C=C\COC(=O)CCCN(CCCC(=O)OC(CCCCCCC)CCCCCCC)C(=O)SCCN(C)C] 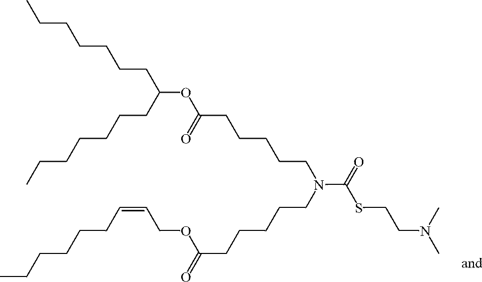 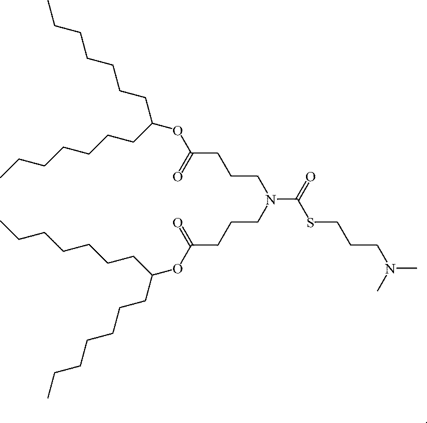
.

40. The pharmaceutical composition of any one of claims 37 to 39 further comprising a pharmaceutically acceptable carrier.

41. The pharmaceutical composition of any one of claims 36 to 40, wherein the composition is formulated for local or systemic administration.

42. The pharmaceutical composition of any one of claims 36 to 41, wherein the composition is formulated for intravenous, subcutaneous, pulmonary, intramuscular, intraperitoneal, dermal, or oral administration.

43. The pharmaceutical composition of any one of claims 36 to 42 comprising a lipid formulation.

44. The pharmaceutical composition of any one of claims 36 to 43, further comprising one or more lipids selected from cationic lipids, anionic lipids, sterols, pegylated lipids, or a combination thereof.

45. The pharmaceutical composition of any one of claims 36 to 44, wherein the composition contains liposomes.

46. The pharmaceutical composition of any one of claims 36 to 45, further comprising a lipid-oligomer nanoparticle comprising a cationic lipid, a cholesterol, a PEG-lipid, and/or a helper lipid.

47. The pharmaceutical composition of claim 46, wherein the lipid-oligomer nanoparticle has a size less than 100 nm.

48. The pharmaceutical composition of claims 46, wherein the cationic lipid is a phospholipid.


SEQUENCE LISTING (14 sequences)

SEQ ID NO: 1 - DNA, 21 bp, Artificial Sequence
  synthetic oligonucleotide
  misc_feature (1)..(1): unlocked uracil
  misc_RNA (2)..(18)
  misc_feature (19)..(21): deoxyribonucleic acid
uagcgacuaaacacaucgctt

SEQ ID NO: 2 - DNA, 21 bp, Artificial Sequence
  synthetic oligonucleotide
  misc_feature (1)..(1): unlocked cytidine
  misc_RNA (2)..(18)
  misc_feature (19)..(21): deoxyribonucleic acid
cagcagcagcagcagcagctt

SEQ ID NO: 3 - DNA, 21 bp, Artificial Sequence
  synthetic oligonucleotides
  misc_RNA (1)..(19)
  misc_feature (20)..(21): deoxyribonucleic acid
gcgauguguuuagucgcuatt

SEQ ID NO: 4 - DNA, 21 bp, Artificial Sequence
  synthetic oligonucleotide
  misc_RNA (1)..(19)
  misc_feature (20)..(21): deoxyribonucleic acid
gcugcugcugcugcugcugtt

SEQ ID NO: 5 - DNA, 21 bp, Artificial Sequence
  synthetic oligonucleotide
  misc_RNA (1)..(2)
  misc_feature (3)..(3): unlocked uridine
  misc_RNA (4)..(19)
  misc_feature (20)..(21): deoxyribonucleic acid
gcugcugcagcugcugcugtt

SEQ ID NO: 6 - DNA, 21 bp, Artificial Sequence
  synthetic oligonucleotide
  misc_RNA (1)..(4)
  misc_feature (5)..(5): unlocked cytosine
  misc_RNA (6)..(19)
  misc_feature (20)..(21): deoxyribonucleic acid
gcugcugcagcugcugcugtt

SEQ ID NO: 7 - DNA, 21 bp, Artificial Sequence
  synthetic oligonucleotide
  misc_RNA (1)..(6)
  misc_feature (7)..(7): unlocked guanine
  misc_RNA (8)..(19)
  misc_feature (20)..(21): deoxyribonucleic acid
gcugcugcagcugcugcugtt

SEQ ID NO: 8 - DNA, 21 bp, Artificial Sequence
  synthetic oligonucleotide
  misc_RNA (1)..(8)
  misc_feature (9)..(9): unlocked adenine
  misc_RNA (10)..(19)
  misc_feature (20)..(21): deoxyribonucleic acid
gcugcugcagcugcugcugtt

SEQ ID NO: 9 - DNA, 21 bp, Artificial Sequence
  synthetic oligonucleotide
  misc_RNA (1)..(9)
  misc_feature (10)..(10): unlocked guanine
  misc_RNA (11)..(19)
  misc_feature (20)..(21): deoxyribonucleic acid
gcugcugcagcugcugcugtt

SEQ ID NO: 10 - DNA, 21 bp, Artificial Sequence
  synthetic oligonucleotide
  misc_RNA (1)..(8)
  misc_feature (9)..(10): unlocked adenine and unlocked guanine
  misc_RNA (11)..(19)
  misc_feature (20)..(21): deoxyribonucleic acid
gcugcugcagcugcugcugtt

SEQ ID NO: 11 - DNA, 21 bp, Artificial Sequence
  synthetic oligonucleotide
  misc_RNA (1)..(9)
  misc_feature (10)..(11): unlocked guanine and unlocked cytosine
  misc_RNA (12)..(19)
  misc_feature (20)..(21): deoxyribonucleic acid
gcugcugcagcugcugcugtt

SEQ ID NO: 12 - DNA, 21 bp, Artificial Sequence
  synthetic oligonucleotide
  misc_RNA (1)..(10)
  misc_feature (11)..(11): unlocked cytosine
  misc_RNA (12)..(19)
  misc_feature (20)..(21): deoxyribonucleic acid
gcugcugcagcugcugcugtt

SEQ ID NO: 13 - DNA, 21 bp, Artificial Sequence
  synthetic oligonucleotide
  misc_RNA (1)..(12)
  misc_feature (13)..(13): unlocked guanine
  misc_RNA (14)..(19)
  misc_feature (20)..(21): deoxyribonucleic acid
gcugcugcagcugcugcugtt

SEQ ID NO: 14 - DNA, 21 bp, Artificial Sequence
  synthetic oligonucleotide
  misc_RNA (1)..(14)
  misc_feature (15)..(15): unlocked uracil
  misc_RNA (16)..(19)
  misc_feature (20)..(21): deoxyribonucleic acid
gcugcugcagcugcugcugtt